SEQUENCE LISTING (1 sequence)

SEQ ID NO: 1 - protein, 20 aa, Artificial Sequence
  BMP-2 peptide
KIPKASSVPTELSAISTLYL

(19) United States
(12) Patent Application Publication
(10) Pub. No.: US 2024/0180850 A1
(43) Pub. Date: Jun. 6, 2024

(54) COMPOSITIONS AND METHODS FOR FABRICATING DEGRADABLE FLOCKED OBJECTS

(71) Applicant: Board of Regents of the University of Nebraska, Lincoln, NE (US)

(72) Inventors: Jingwei Xie, Omaha, NE (US); ALEC MCCARTHY, Omaha, NE (US)

(21) Appl. No.: 18/283,606

(22) PCT Filed: Apr. 14, 2022
(86) PCT No.: PCT/US22/24802

§ 371 Date: Sep. 22, 2023

Related U.S. Application Data
(60) Provisional application No. 63/174,686, filed on Apr. 14, 2021.

(51) Int. Cl. A61K 9/70; A61K 33/38; A61K 35/66; A61K 47/34; A61K 47/36; A61K 47/44; A61P 7/04; A61P 19/08

(52) U.S. Cl. CPC A61K 9/7007; A61K 33/38; A61K 35/66; A61K 47/34; A61K 47/36; A61K 47/44; A61P 7/04; A61P 19/08

(57) ABSTRACT

Flocked substrates are provided as well as methods of use thereof and methods of making.

This application claims priority under 35 U.S.C. § 119(e) to U.S. Provisional Patent Application No. 63/174,686, filed Apr. 14, 2021. The foregoing application is incorporated by reference herein.

This invention was made with government support under Grant Nos. R01 GM123081 and R01 GM138552 awarded by the National Institutes of Health. The government has certain rights in the invention.

### FIELD OF THE INVENTION

This application relates to the fields of microfiber structures. More specifically, this invention provides microfiber flocked structures, methods of synthesizing microfiber flocked structures and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Electrostatic flocking, a textile engineering technique that usually comprises three main components (flock fibers, an adhesive, and a substrate) uses an electrostatic field to launch short microfibers (flock fibers) toward an adhesive-covered substrate, creating a layer of vertically-aligned fibers perpendicular to the substrate (Vellayappan, et al. (2015) RSC Adv., 5:73225; Gokarneshan (2018) Curr. Trends Fashion Technol. Textile Eng., 4:555630). Generally, flocking can be regarded as a surface modification technique used to decorate object surfaces or planar textiles. Application of flock fibers (short polymeric microfibers with proprietary electrostatic surface finishes) to an object increases regional mechanical strength, generates anisotropic surfaces with high surface areas and porosities, and allows for surface functionalization based on the type of fiber used. For example, flocking has been used to create marine antifouling surfaces, solar-driven steam generators, microfluidic chips for self-coalescing flow, elastomeric thermal interface and composite materials, stretchable fabric-based electrodes, shock-absorbing materials, diagnostic swabs, and tissue engineering scaffolds (Phillippi, et al. (2001) Aquaculture 195:225; Xu, et al. (2021) J. Colloid Interface Sci., 588:9; Guo, et al. (2021) Adv. Sustainable Syst., 5:2000202; Tu, et al. (2019) Small 15:1902070; Hitzbleck, et al. (2013) Adv. Mater., 25:2672; Uetani, et al. (2014) Adv. Mater., 26:5857; Yu, et al. (2019) J. Mater. Sci.: Mater. Electron., 30:10233; Li, et al. (2020) Chem. Eng. J., 390:124442; Lewis, et al. (2018) Text. Res. J., 88:392; Fodor, et al. (2018) Compos. Struct., 207:677; Dalmaso, et al. (2008) J. Pharm. Sci. Technol., 62:191; McCarthy, et al. (2021) Nano Lett., 21:1508; Probst, et al. (2010) Appl. Environ. Microbiol., 76:5148; Gossla, et al. (2016) Acta Biomater., 44:267; Steck, et al. (2010) Tissue Eng., 16:3697; Walther, et al. (2012) Materials 5:540; Walther, et al. (2007) Text. Res. J., 77:892; Tonndorf, et al. (2018) Text. Res. J, 88:1965; Balasubramanian, et al. (2015) Mater. Lett., 158:313). Though introduced to tissue engineering more than a decade ago, progress with flocked scaffolds has been limited due to restrictive electrical conductivity requirements, inability to generate individual flock fibers, and a lack of understanding in approach and synthesis.

Previous studies using flocked scaffolds in tissue engineering have exclusively investigated their use as osteochondral scaffolds. While some progress has been made toward using fully biodegradable flocked scaffolds, flocking in tissue engineering remains largely underdeveloped (Steck, et al. (2010) Tissue Eng., 16:3697; Walther, et al. (2012) Materials 5:540; Walther, et al. (2007) Text. Res. J., 77:892). For example, flocked scaffolds developed by Gossla et al. were fully degradable, composed entirely of chitosan (CHS), and able to sustain cell proliferation (Gossla, et al. (2016) Acta Biomater., 44:267). However, the exclusive use of CHS in these scaffolds dramatically limited their practical applications. For instance, it may be favorable to utilize substrates or fibers that degrade at different rates, particularly for applications in wound regeneration and integumentary tissue engineering (Chang, et al. (2019), Semin. Plast. Surg., 3:33). Perhaps one of the most prominent bottlenecks limiting the use of electrostatic flocking in tissue engineering is the requirement of fibers to be able to accumulate charges, an atypical characteristic for some of the common polymers used in tissue engineering (Zenkiewicz, et al. (2015) Polym. Test., 42:192).

These limitations necessitate the development of improved methods for electrostatic flocking.

### SUMMARY OF THE INVENTION

In accordance with the instant invention, flocked substrates are provided. In certain embodiments, the flocked substrates comprise microfibers attached or flocked on a surface of the substrate, optionally wherein the surface of the substrate is coated with an adhesive such as a biodegradable adhesive. In certain embodiments, the microfibers comprise a polymer and a conductive filler. In certain embodiments, the conductive filler is deposited on the surface of the polymer. In certain embodiments, the microfiber comprises the conductive filler throughout the microfiber. In certain embodiments, the polymer is a non-conductive polymer or an insulative polymer. In certain embodiments, the conductive filler comprises a metal or metal nanoparticles. In certain embodiments, the conductive filler comprises a salt. In certain embodiments, the conductive filler comprises nanoparticles. In certain embodiments, the flocked substrate may comprise a material that enhances water absorption, such as gelatin, chitosan, or collagen. In certain embodiments, the flocked substrates are crosslinked, such as by glutaraldehyde or a crosslinker. In certain embodiments, the flocked substrates are mineralized. The flocked substrates may also comprise one or more agents or compounds such as therapeutic agents. In certain embodiments, the flocked substrates comprise cells and/or tissue. Methods of synthesizing the flocked substrates of the instant invention are also provided.

In accordance with another aspect of the instant invention, methods of using the flocked substrates are provided. For example, the flocked substrates may be used to enhance wound healing, build tissue constructs, promote tissue regeneration (e.g., bone regeneration), reduce, inhibit, prevent, and/or eliminate infection, local delivery of drugs, and/or inhibit bleeding.

### BRIEF DESCRIPTION OF THE DRAWINGS

FIG. 1A provides a schematic illustrating the fabrication process of flocked scaffolds. PCL and AgNPs were dissolved/dispersed in the organic solvent. The AgNP/PCL solution was wet spun into a coagulation bath and collected on a take up roller. Fibers were cut and separated with sonication and bubbling. Fibers were sieved onto the charging electrode and electrostatically flocked toward an adhesive-covered substrate. Flocked scaffolds were removed and crosslinked. The bottom panels are the corresponding SEM images and photograph. FIG. 1B provides a graph of the diameters of microfibers spun at different extrusion rates. FIG. 1C provides a graph of the diameters of microfibers spun at different take-up rates. Data is presented as the mean±standard deviation at each flow rate/take up rate (n=10).

FIGS. 2A-2E provide the characterizations of electrical conductivity, morphology, and Ag+ content and release of PCL microfibers with different AgNP loadings. FIG. 2A: Theoretical cross-section view of PCL microfibers at different AgNP concentrations (top), photograph of microfibers with increasing (left to right) concentration of AgNPs (middle), and different concentration AgNP microfibers after cutting (bottom). FIG. 2B: Estimated conductivities using the Percolation Theory model for PCL microfibers loaded with 70 nm diameter AgNPs (conductivity threshold for flocking indicated with arrow). FIG. 2C: Relationship between loaded AgNP and detected Ag⁺ contained within fibers. Data is presented as mean±standard deviation (n=3) curve fit with simple linear regression. Significance was determined using a two-way ANOVA with Tukey's multiple comparisons post hoc test. FIG. 2D: Ag⁺ release from microfibers with different AgNP concentrations over 28 days incubating in PBS. Data is presented as mean±standard deviation (line graph and mean±standard deviation (bar graph) (n=9)) and significance was calculated using a two-way ANOVA with Tukey's multiple comparison post hoc test. Significance was denoted as follows: (*p<0.05, **** p<0.0001, no pairwise bar p>0.05). FIG. 2E shows the elucidation of fiber charging mechanisms. Comparing flock yield between 0.5% AgNP/PCL fibers and surface-coated Rayon fibers at different relative humidity (left). Statistical comparison between flock yields of AgNP/PCL and Rayon fibers at different humidity values (right). Data is presented as the mean±standard deviation (n=3). Significance was calculated using a two-way ANOVA with Tukey's multiple comparisons post-hoc test.

FIGS. 3A-3H show the characterization of flocked scaffolds made of 0.5% AgNP-loaded PCL microfibers. SEM images showing side view (FIG. 3A), interface between flock fiber and chitosan adhesive (FIG. 3B), top view (FIG. 3D), and magnified side view of flocked scaffolds (FIG. 3E). FIG. 3C: Relationship between flock time and fiber density. FIG. 3F: Calculated scaffold porosity. Data in FIGS. 3C and 3F are presented as the mean±standard deviation (n=8) and significance was calculated using a one-way ANOVA with Tukey's multiple comparison post hoc test. FIG. 3G provides the distribution of Rayon flock fiber orientation. FIG. 3H provides the distribution of 0.5% AgNP/PCL flock fiber orientation. Data for FIGS. 3G and 3H are presented as a continuous frequency of fiber orientation and distributions were compared using a Kolmogorov-Smirnov test (K-S D=0.386).

FIGS. 4A-4C provide images of surface flocked biomedical substrates and self-folding conduit. FIG. 4A: Flocked 3D printed multilayered meshes. Photograph and CAD file for 3D printed PCL/hydroxyapatite circular mesh substrate (left). SEM images of mesh substrate before (middle) and after (right) electrostatic flocking of 0.5% AgNP/PCL microfibers. FIG. 4B: Flocked electrospun nanofiber membrane. Uncoated PCL nanofiber substrate (left). SEM image of the PCL mesh before (middle) and after (right) electrostatic flocking of 0.5% AgNP/PCL microfibers. FIG. 4C: Heat-responsive triphasic conduit. PCL nanofiber mat after flocking with PLA microfibers (top left). Heat application to PLA-flocked PCL nanofiber mat to initiate curling (top middle). Photograph of PLA-flocked PCL nanofiber mat after self-curling and fixation (top right). SEM image of PLA-flocked PCL nanofiber tube edge (bottom left) and cross-section (bottom middle). SEM image highlighting the micro-to-nanofiber interface separated by an adhesive layer (bottom right).

FIGS. 5A-5C show flocked scaffolds assembled from different microfiber compositions. FIG. 5A: SEM images of individual microfibers of different polymeric/additive compositions (top) and flocked scaffolds with each different fiber composition (bottom). FIG. 5B: The quality of anisotropy by distribution of fiber orientation of each kind of fiber. Data is presented as continuous frequencies of fiber orientation. FIG. 5C provides a heatmap displaying the percentage of AgNP-loaded PCL, PLA, and PLGA fibers and 1.0% w/v FeCl₃, 1% w/v HTAB, 1% w/v NaCl, 0.75% w/v SPION, 1.0% w/v zinc powder, and pure PCL fibers lifted during electrostatic flocking. Data is displayed as the mean value (n=3) and corresponds with the heat map legend for mean % lifted. Significance was determined using a one-way and two-way (main effects model) ANOVA with Tukey's multiple comparison post hoc test.

FIGS. 6A-6C provide the mechanical characterization of flocked scaffold components. FIG. 6A: Fiber tensile test. Photograph of tensile testing set up (multiple fibers were loaded onto grips for visibility). Schematic illustrating tensile test and fiber necking. Stress-strain curves at different AgNP loading concentrations. Data is presented as nonsmoothed stress-strain curves (n=4). Ultimate tensile stress (UTS) for PCL microfibers loaded with varying AgNP concentrations. Data is presented as the mean±standard deviation (n=4) and significance was determined using one-way ANOVA with Tukey's multiple comparison post hoc test. FIG. 6B: Substrate tear test. Trouser tear test set up and schematic. Tear test results following six iterative transverse tears on a chitosan substrate. Ultimate tearing stress at each transverse tear iteration. Data is presented as the individual values on each continuous tear iteration (n=3) and significance was determined using one-way ANOVA with Tukey's multiple comparison post hoc test. FIG. 6C: Abrasion resistance test. Picture and schematic showing the thumb test for abrasion resistance. Mass change of 0.5% AgNP/PCL after 100 abrasive cycles Data is presented as the mean±standard deviation (n=3) and significance was determined using one-way ANOVA with Tukey's multiple comparison post hoc test. Degradation rates of different substrates in simulated body fluid (SBF) (T=thermally crosslinked, Ga=glutaraldehyde crosslinked). Data is represented as the mean±standard deviation (n=10) and significance was determined using two-way ANOVA with Tukey's multiple comparison post hoc test. Significance was denoted as follows: (*p<0.05, ** p<0.01, **** p<0.0001, no pairwise bar p>0.05). FIGS. 6D-6F show the biological activity of flocked scaffolds. Cell proliferation (FIG. 6D) and viability (FIG. 6E) after 1, 3, 5, and 7 days of culture. Data is presented as the mean±standard deviation (n=5) and significance was determined using a one-way ANOVA with Tukey's multiple comparison post-hoc test. FIG. 6F: CFU counts at each AgNP/PCL concentration. Data is presented as the mean±standard deviation (n=3) and significance was determined using one-way ANOVA with Tukey's multiple comparison post-hoc test. Significance was denoted as follows: (*p<0.05, ** p<0.01, *** p<0.001, **** p<0.0001, no pairwise bar p>0.05).

FIGS. 7A-7F shows the in vitro and in vivo biological response. FIG. 7A: Cell infiltration into 0.5% AgNP/PCL flocked scaffolds. Maximum intensity actin/DAPI confocal microscopy images of rBMSCs seeded onto 0.5% AgNP/PCL flocked scaffolds after 1, 14, and 28 days. 3D orthogonal view of actin/DAPI stained rBMSCs cells after seeding onto 0.5% AgNP/PCL flocked scaffolds and culturing for 1, 14, and 28 days. FIG. 7B: Average distance of tissue layer migration over 28 days. Data is presented as the mean±standard deviation (n=5) and significance was determined using a one-way ANOVA with Tukey's multiple comparison post hoc test. FIG. 7C: Subcutaneous implantation of 0.5% AgNP/PCL scaffolds with low, medium, and high fiber densities in Sprague-Dawley rats (n=4). Trichome (10×) images (left) of surrounding tissue (lines indicate flocked scaffolds), trichome (40×) images (middle) indicating newly formed blood vessels (arrows), and H&E (10×) staining (right) of surrounding tissues (lines indicate flocked scaffolds). Scale bar=1 mm and 200 μm for 10× and 40× images, respectively. FIG. 7D Scaffold positioning during surgical implantation (fibers facing outward). Subcutaneous view of flocked scaffolds. Approximate scaffold placement on rats. FIG. 7E: Angiogenesis around low-, medium-, and high-density flocked scaffolds. Data is presented as the mean±standard deviation (n=4) and significance was determined using a one-way ANOVA with Tukey's multiple comparison post hoc test (** p<0.01). FIG. 7F: Host cell penetration (measured as average migration from scaffold edge) into low-, medium-, and high-density flocked scaffolds. Data is presented as the mean±standard deviation (n=4) and significance was determined using a one-way ANOVA with Tukey's multiple comparison post hoc test. Significance was denoted as follows: (*p<0.05, ** p<0.01, **** p<0.0001, no pairwise bar p>0.05).

FIGS. 8A-8F provide the characterization of fiber fabrication. FIG. 8A: SEM images of PCL microfibers prepared at increased take-up rates. FIG. 8B: Curve-fit for microfiber diameter estimation for wet spinning. FIG. 8C: Comparison of average microfiber diameters. FIG. 8D: SEM images of nanofiber yarns rolled from different diameter nanofiber strips. FIG. 8E: Curve-fit guide for nanofiber yarn diameter estimation. FIG. 8F: Comparison of average nanofiber yarn diameters. NFY: nanofiber yarns.

FIGS. 9A-9D show the characterization of microfibers and nanofiber yarns. FIG. 9A: Absorption ratios of each fiber type after immersion in a BSA solution. FIG. 9B: BSA release profiles of each fiber type. FIG. 9C: Force-displacement curves of each fiber type. FIG. 9D: Maximum break forces and maximum strains of each fiber type. NFY: nanofiber yarns. MF: microfibers.

FIGS. 10A-10F show that the introduction of salt via salt bath washing increases flocking yields over a range of relative humidity. FIG. 10A: SEM image of a salt-covered (arrows) microfiber (MF) after flocking. FIG. 10B: SEM image of a flock fiber after washing with H₂O. FIG. 10C: SEM of a salt-covered nanofiber yarn (NFY) after flocking. FIG. 10D: SEM of a salt-covered nanofiber yarn (NFY) after rinsing with H₂O. FIG. 10E: The flock yield of MFs prepared with two different salt implementing strategies. FIG. 10F: The resulting yield of NaCl-rinsed and untreated MFs and NFYs at a range of humidities.

FIGS. 11A-11H show flock fibers and scaffold characterization. FIG. 11A: SEM images of microfibers (MFs). FIG. 11B: SEM images of flocked MF scaffolds. FIG. 11C: SEM images of nanofiber yarns (NFYs). FIG. 11D: SEM images of flocked NFY scaffolds. FIG. 11E: Anisotropy measurements of rayon (control), MFs, and NFYs. FIG. 11F: Mass loss from abrasive cycles of MFs and NFYs during rub testing. FIG. 11G: Compression curves of NFY and MF scaffolds undergoing 50% displacement. FIG. 11H: Average compressive force lost between the first and fourth compressive cycle.

FIG. 12A-12C show HaCaT proliferation and migration. FIG. 12A: Normalized fluorescence intensity of HaCaT-seeded scaffolds at 3, 5, and 7 days. FIG. 12B: Depth mapping of HaCaTs on the flocked scaffolds at 3, 5, and 7 days. FIG. 12C: Leading average cell migration measurements.

FIG. 13A-13G shows low- and high-density flocked scaffolds for wound healing. FIG. 13A Surgical strategy schematic. Flock fibers are positioned facing the wound bed and the chitosan/gelatin substrate is flush with the wound edge. FIG. 13B: Splints are glued and sutured into place to prevent wound contraction. H&E staining of wounds after 7 days (FIG. 13C) and 14 days (FIG. 13D) of no treatment, low-density flock scaffold treatment, and high-density flock scaffold treatment. FIG. 13E: Re-epithelialization measured as the fraction of epithelium over the defect site. FIG. 13F: New vessels formed within the wounds. FIG. 13G: Collagen deposition within the wound measured via integrated density in color-split trichrome stained images. Control: without treatment; Low: low-density flocked scaffolds; High: high-density flocked scaffolds.

FIGS. 14A-14L show the mechanical analysis of artificial vertebral disc (AVD) undergoing cyclic compressive loads. FIG. 14A: Photograph of AVD situated on L5 model during sizing. Representative side (FIG. 14B) and top (FIG. 14C) view of AVD in situ. Schematic and cross-sectional microscopic image of pure PDMS AVD (FIG. 14D), isotropic fiber-reinforced AVD (FIG. 14E), and flock fiber reinforced AVD FIG. 14F). FIG. 14G: Full stress-strain curve of each AVD model. FIG. 14H: Stress curves during the first 3 minutes of compression (ramping). FIG. 14I: Last 10 cyclic compressive loads to display waveform stress recovery. Dissipation energy during the first 10 (FIG. 14J) and last 10 (FIG. 14K) compressive cycles and their change between cycle sets (FIG. 14L).

FIGS. 15A-15C show that flocked AgNP/PCL fiber and chitosan adhesive/substrate scaffolds demonstrate significant cranial bone regeneration effect in a rat cranial model. FIG. 15A: Micro computed tomography images of rat cranial bone defects untreated and treated with low and high-density flocked scaffolds for 7 weeks. FIG. 15B: Scaffold orientation in situ (substrate flush with dura mater). FIG. 15C: Graph comparing recovered bone volume (%) between each group (P<0.05, n=4).

### DETAILED DESCRIPTION OF THE INVENTION

Electrostatic flocking, a textile engineering technique, uses Coulombic driving forces to propel conductive microfibers toward an adhesive-coated substrate, leaving a forest of aligned fibers. Though an easy way to induce anisotropy along a surface, this technique is limited to microfibers capable of accumulating charge. One method used to induce conductivity in electrostatically insulative materials is based on the percolation theory, which establishes a tunneling network of conductive filling particles to achieve bulk material semiconductivity/conductivity (Kim, et al. (2016) Sci. Rep, 6:34632; Gonon, et al. (2006) J. Appl. Phys., 99:024308; Lee, et al. (2018) J. Korean Phys. Soc., 73:152). Herein, it was hypothesized that by applying principles from the percolation theory, flock fibers could be created by loading polymeric, wet-spun microfibers with conductive material such as nanoparticles to ultimately generate anisotropic and biphasic scaffolds for biomedical applications. Herein, a flexible method was established to create flocked scaffolds using a variety of biomedical polymers and conductive treatments, generating a first-of-kind biomedical flock fiber toolbox. Application flexibility was demonstrated by creating flocked scaffolds with both electrospun nanofibers and 3D-printed substrates. Noting the ability to retain anisotropic alignment under substrate deformation, a self-forming, biphasic conduit with nano-/micro-hierarchical structures was developed. Finally, the morphological, mechanical, and biological properties (in vitro and in vivo) of novel 0.5% silver nanoparticle (AgNP)/poly(ϵ-caprolactone) PCL flocked scaffolds was evaluated for regenerative medicine applications.

In accordance with the instant invention, flocked substrates and methods of synthesizing a flocked substrate are provided. In certain embodiments, the flocked substrates are anisotropic. As used herein, the term “flocking” refers to the process of depositing fibers, particularly short fibers, onto a substrate surface. Generally, the deposited fibers are attached to the substrate such that the fibers are substantially perpendicular to the surface of the substrate. In certain embodiments, the fibers are oriented at an angle (e.g., average or mean angle) of at least (or greater than) about 60 degrees, about 65 degrees, about 70 degrees, about 75 degrees, about 80 degrees, or about 85 degrees to 90 degrees relative to the surface of the substrate (e.g., as measured at the attachment to the substrate). The concentration or density of fibers on the substrate can be varied as desired. For example, a flocked swab may have a high flock density in order to achieve a high surface-to-volume ratio. If a porous anisotropic biological material is to be mimicked, lower flock density may be preferred as to better allow cells to infiltrate into and proliferate throughout flock fibers. In certain embodiments, the density of fibers on the surface of the substrate is about 1 to about 1000 fibers/mm², about 5 to about 500 fibers/mm², about 10 to about 250 fibers/mm², about 10 to about 200 fibers/mm², about 50 to about 150 fibers/mm², or about 100 fibers/mm². In certain embodiments, the density of fibers on the surface of the substrate is greater than about 10 fibers/mm², greater than about 25 fibers/mm², greater than about 50 fibers/mm², greater than about 75 fibers/mm², or greater than about 100 fibers/mm². In certain embodiments, the density of fibers on the surface of the substrate is less than about 100 fibers/mm², less than about 75 fibers/mm², less than about 50 fibers/mm², less than about 25 fibers/mm², or less than about 10 fibers/mm². Typically, only one surface or side or face of the substrate is flocked and the entire area of the one surface side/face is flocked. However, the present invention encompasses flocked substrates wherein the entire surface of the substrate is flocked or any percentage (e.g., at least 1%, at least 5%, at least 10% or more) of any surface or side or face of the substrate is flocked.

The substrate of the flocked substrate can be any substrate and may compose any material. For example, the substrate may comprise polymers, fabric, ceramic, and/or metals. While the term “substrate” is used herein for simplicity, the substrate may also be referred to as a scaffold. Typically, the substrate is selected for the intended purpose of the finished flocked substrate. The substrate may be shaped to the desired shape prior to flocking. Indeed, the morphology of the substrate can be controlled (e.g., during synthesis). For example, the substrates can be, without limitation: solid, nanofibrous, porous, layered or combinations thereof. In certain embodiments, the substrate is flat or planar. In certain embodiments, the substrate is a film. In certain embodiments, the substrate is a bandage, swab (e.g., for specimen collection), swab applicator, tissue scaffold, or nanofiber material. In certain embodiments, the flocked substrate is applied or adhered to another substrate or scaffold to produce a final product. For example, the flocked substrate can be added or adhered to an artificial vertebral disc or lumbar disc replacement. Alternatively, the microfibers may be flocked directly onto the final substrate or scaffold.

The substrate may be synthesized by any method. The substrate may be made of any material. In certain embodiments, the methods of the instant invention comprise synthesizing the substrate prior to flocking. In certain embodiments, the substrate is synthesized by three dimensional (3D) printing. In certain embodiments, the substrate is synthesized by electrospinning (e.g., nanofiber mats). In certain embodiments, the substrate is synthesized by thin-film casting. In certain embodiments, the substrate comprises a polymer as defined hereinbelow for the microfiber. In certain embodiments, the substrate comprises an adhesive as defined hereinbelow. In certain embodiments, the substrate and the microfiber comprise the same polymer(s). In certain embodiments, the substrate comprises polydimethylsiloxane. In certain embodiments, the substrate is biodegradable. In certain embodiments, the substrate is biocompatible. In certain embodiments, the substrate is not biodegradable. In certain embodiments, the substrate comprises polycaprolactone (PCL), poly(lactide) (PLA), and/or poly(lactic-co-glycolic acid) (PLGA). In certain embodiments, the substrate comprises chitosan and/or gelatin. In certain embodiments, the substrate comprises a biomaterial. In certain embodiments, the substrate comprises collagen, hydroxyapatite, chitosan, and/or cellulose.

In certain embodiments, the method of the instant invention comprises flocking microfibers onto a substrate. In certain embodiments, the substrate is coated with an adhesive. In certain embodiments, the substrate is an adhesive. In certain embodiments, the method comprises applying or coating an adhesive to a substrate and flocking microfibers onto the adhesive coated substrate. The methods of the instant invention allow for control over the anisotropy, porosity, compressive strength, and/or degradative rate of the flocked substrate.

Generally, the microfibers comprise a polymer and a conductive filler. Microfibers of the instant invention have a diameter (e.g., average diameter) greater than 1 μm and less than about 2 mm, typically less than about 1 mm. While the flocked substrates are described herein as comprising microfibers, the instant invention also encompasses flocked substrates comprising nanofibers (a diameter (e.g., average diameter) less than about 1 μm but greater than 1 nm), either with or without microfibers. In certain embodiments, the microfibers have a diameter (e.g., average diameter) from about 1 μm to about 1000 μm, about 1 μm to about 750 μm, about 1 μm to about 500 μm, about 1 μm to about 400 μm, about 1 μm to about 300 μm, about 1 μm to about 250 μm, about 1 μm to about 200 μm, about 1 μm to about 150 μm, about 1 μm to about 100 μm, about 5 μm to about 100 μm, about 5 μm to about 90 μm, about 5 μm to about 80 μm, about 5 μm to about 75 μm, about 10 μm to about 75 μm, about 10 μm to about 70 μm, about 10 μm to about 65 μm, about 10 μm to about 50 μm, or about 10 μm to about 30 μm. In certain embodiments, the microfibers have a diameter (e.g., average diameter) greater than about 5 μm or greater than about 10 μm. In certain embodiments, the microfibers have a diameter (e.g., average diameter) less than about 100 μm.

The microfibers to be flocked onto the substrate may be short segments. The microfiber segments of the instant invention can be fabricated by any method. In certain embodiments, the microfibers are monofilament microfibers. In certain embodiments, the microfibers are multifilaments or yarns (e.g., nanofiber yarns such as electrospun nanofiber yarns). In certain embodiments, the microfiber segments are derived from longer microfibers (e.g., microfiber tow), such as by cutting, mechanical cutting, cryocutting and the like. In certain embodiments, the microfiber segments have a median or mean length of about 1 mm. In certain embodiments, the microfiber segments have a median or mean length of about 100 μm to about 100 mm, about 100 μm to about 50 mm, about 100 μm to about 10 mm, about 250 μm to about 10 mm, about 200 μm to about 5 mm, about 500 μm to about 10 mm, about 500 μm to about 5 mm, or about 500 μm to about 2.5 mm.

As stated hereinabove, the microfibers generally comprise at least one polymer and at least one conductive filler. The microfibers of the instant invention may comprise any polymer. In certain embodiments, the polymer is a substantially insulative polymer (e.g., electrically insulative polymer). In certain embodiments, the polymer is a substantially non-conductive polymer. In certain embodiments, the polymer—without a conductive filler has a flock yield of less than 50%, less than 40%, less than 30%, or less than 25%. In certain embodiments, the polymer is biocompatible. In certain embodiments, the polymer is biodegradable. The polymer may by hydrophobic, hydrophilic, or amphiphilic. In certain embodiments, the polymer is hydrophobic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 80,000, about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid). In certain embodiments, the polymer is an amphiphilic block copolymer. In certain embodiments, the amphiphilic block copolymer comprises hydrophilic poly(ethylene oxide) (PEO) and hydrophobic poly(propylene oxide) (PPO). In a particular embodiment, the polymer comprises a poloxamer or an amphiphilic triblock copolymer comprising a central hydrophobic PPO block flanked by two hydrophilic PEO blocks (i.e., an A-B-A triblock structure). In certain embodiments, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4.

Further examples of polymers for use in the microfibers of the instant invention include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium aliginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA).

In certain embodiments, the polymer comprises polymethacrylate, poly vinyl phenol, polyvinylchloride, cellulose, polyvinyl alcohol, polyacrylamide, poly(lactic-co-glycolic acid) (PLGA), collagen, polycaprolactone, polyurethanes, polyvinyl fluoride, polyamide, silk, nylon, polybenzimidazole, polycarbonate, polyacrylonitrile, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, starch-acrylonitrile co-polymers, bioactive glass, and/or combinations of two or more polymers. Multiple polymers may be mixed to form the microfibers. The polymers may be mixed in equal ratios or various ratios depending on the desired properties of the microfibers.

In certain embodiments, the polymer comprises poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly (tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

In certain embodiments, the polymer comprise polycaprolactone (PCL), poly(lactide) (PLA), and/or poly(lactic-co-glycolic acid) (PLGA).

The conductive filler of the microfiber may be any conductive material. While the term “conductive filler” is used herein, the conductive filler may also be referred to as a conductive material. In certain embodiments, the conductive filler is contained throughout the microfiber. In certain embodiments, the conductive filler is contained on the surface of the microfiber (e.g., deposited on the microfiber (e.g., such as salt crystals)). In certain embodiments, the conductive filler is nanoparticles. The nanoparticles may have a diameter (e.g., average diameter) less than 1 μm. In certain embodiments, the nanoparticles have a diameter (e.g., average diameter) of about 1 nm to about 1 μm, about 1 nm to about 500 nm, about 1 nm to about 400 nm, about 1 nm to about 300 nm, about 1 nm to about 250 nm, about 1 nm to about 200 nm, about 10 nm to about 200 nm, about 10 nm to about 150 nm, about 20 nm to about 150 nm, about 30 nm to about 100 nm, about 40 nm to about 100 nm, about 50 nm to about 100 nm, about 50 nm to about 90 nm, about 60 nm to about 80 nm, or about 70 nm. In certain embodiments, the nanoparticles have a diameter (e.g., average diameter) greater than about 5 nm, greater than about 10 nm, greater than about 15 nm, greater than about 20 nm, or greater than about 25 nm. The conductive filler may be a conductive metal (e.g., nanoparticles of a conductive metal). Conductive fillers include, without limitation: hexadecyltrimethylammonium bromide (HTAB), salts (e.g., salt ions), sodium chloride (NaCl), silver, zinc, ferric chloride (FeCl₃), iron oxide, graphene oxide, copper, gold, and aluminum. In certain embodiments, the conductive filler is hexadecyltrimethylammonium bromide (HTAB), ferric chloride (FeCl₃), silver (e.g., AgNP), zinc (e.g., zinc powder), sodium chloride (NaCl), or superparamagnetic iron oxide nanoparticles (SPIONS). In certain embodiments, the conductive filler is silver nanoparticles. In certain embodiments, the conductive filler is an ionizable salt such as sodium chloride.

In certain embodiments, the methods of the instant invention further comprise synthesizing the microfibers. The microfibers of the instant invention can be fabricated by any method. Methods for generating the microfibers used in the present invention include, but are not limited to, wet spinning, wet electrospinning, melt spinning, and electrospinning. In certain embodiments, the microfibers are generated by wet spinning. In certain embodiments, the microfibers are generated by electrospinning. Generally, “wet spinning” refers to a process of preparing polymer fibers in which a stream of a solution comprising a polymer is ejected or extruded into a liquid bath containing a non-solvent (e.g., coagulation bath), resulting in the formation of a polymer fiber. Typically, the polymer fiber (e.g., polymer tow) is subjected to a tensile force—such as a rolling drum—that draws the polymer fiber out of the liquid bath.

In certain embodiments, the method of synthesizing the microfibers comprises dissolving or mixing the polymer and the conductive filler and then wet spinning or electrospinning microfibers. In certain embodiments, the method of synthesizing the microfibers comprises wet spinning or electrospinning microfibers and salt washing or rinsing the microfibers. For example, the synthesized microfibers may be placed in a salt bath, an ionic liquid, or liquid comprising a conductive filler. In certain embodiments, the method of synthesizing the microfibers comprises wet spinning or electrospinning microfibers and depositing (e.g., spraying) the conductive filler on the surface of the microfiber.

In certain embodiments, the microfibers are plasma treated. For example, the microfibers can be air plasma treated or plasma treated with oxygen.

The resultant microfibers may then be cut or broken into microfiber segments as described hereinabove. For example, the methods may further comprise cutting or breaking the produced microfibers by cutting, mechanical cutting, cryocutting or the like. In certain embodiments, the methods further comprise separating the microfiber segments after cutting. For example, the cut microfiber segments may be separated by sieving (e.g., mechanical sieving), sonicating or ultrasonicating, shaking, and/or exposing to bubbling.

In certain embodiments, the methods of the instant invention further comprise applying an adhesive to the surface of the substrate. The microfiber segments will be flocked onto the surface of the substrate and the adhesive aids in retention of the microfiber segments on the surface of the substrate. The adhesive may be applied to the surface of the substrate by any means including, without limitation, spray coating, dip coating, physically spreading the adhesive on the substrate, and the like. In certain embodiments, the adhesive is biodegradable. In certain embodiments, the adhesive is biocompatible. In certain embodiments, the adhesive is a medical adhesive. Examples of medical adhesives include, without limitation: acrylic adhesive, cyanoacrylate adhesive, epoxy adhesive, polyurethane adhesive, silicone adhesive, and polyethylene glycol. In certain embodiments, the adhesive comprises alginate, hyaluronic acid, and/or collagen. In certain embodiments, the adhesive comprises gelatin, chitosan, tannic acid, albumin, collagen, fibrin, and/or polysaccharide. In certain embodiments, the adhesive comprises gelatin, chitosan, and/or tannic acid. In certain embodiments, the adhesive comprises gelatin and/or chitosan.

As stated hereinabove, the microfiber segments are flocked onto the surface of the substrate, optionally coated in an adhesive. In certain embodiments, the substrate is an adhesive. The microfiber segments may be flocked onto the surface of the substrate using electrostatic flocking or pneumatic flocking. In certain embodiments, the microfiber segments are flocked onto the surface of the substrate using electrostatic flocking (e.g., DC electrostatic). In certain embodiments, the electrostatic flocking is performed with increasing voltage (e.g., from 0 kV to 60 kV or 30 to 60 kV). In certain embodiments, the flocking is performed with a relative humidity of about 20% to about 60% or about 40% to about 50%. Optionally, unattached microfibers may be removed from the flocked substrate (e.g., by application of air or a vacuum or by rinsing with water).

After flocking, the flocked substrate can be given multiple different treatments. In certain embodiments, the methods of the instant invention further comprise drying, thermally curing, crosslinking, washing, and/or sterilizing the flocked substrate.

In certain embodiments, the methods further comprise drying the flocked substrate. For example, the flocked substrate may be dried at temperature above room temperature and below the melting temperature of the components of the flocked substrate. In certain embodiments, the flocked substrate is dried at about 40° C. to about 80° C., about 50° C. to about 70° C., or about 60° C. In certain embodiments, the flocked substrate is dried for less than about 15 minutes, less than about 10 minutes, or about 5 minutes.

In certain embodiments, the methods further comprise curing the flocked substrate. The flocked substrate may be cured after drying. In certain embodiments, the flocked substrate is thermally cured such as at room temperature for about 1-3 hours.

In certain embodiments, the methods further comprise crosslinking the flocked substrate. The flocked substrate may be crosslinked after drying and/or curing the flocked substrate. Crosslinking may be done using a variety of techniques including thermal crosslinking, chemical crosslinking, and photo-crosslinking. For example, the flocked substrates of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457, Bigi, et al., Biomaterials (2002) 23:4827-4832; Zhang, et al, Biomacromolecules (2010) 11:1125-1132; incorporated herein by reference). The crosslinker may be a bifunctional, trifunctional, or multifunctional crosslinking reagent. For example, the crosslinker may be 1-ethyl-3-(3-dimethylaminopropyl)carbodiimide hydrochloride (EDC). In certain embodiments, the flocked substrate may be crosslinked with a chemical crosslinker such as formaldehyde, paraformaldehyde, acetaldehyde, glutaraldehyde, or the like. In certain embodiments, the flocked substrate is crosslinked with glutaraldehyde (e.g., 1% glutaraldehyde).

In certain embodiments, the flocked substrate (e.g., the final flocked substrate) is washed. The flocked substrates may be washed with water, deionized H₂O, saline, a buffer, a carrier, or a pharmaceutically acceptable carrier. In certain embodiments, the flocked substrate is washed (e.g., with water) after flocking—such as to remove salt from the surface of the microfibers.

In certain embodiments, the flocked substrate (e.g., the final flocked substrate) is sterilized. The flocked substrate can be sterilized using various methods including, without limitation: gas sterilization (e.g., by treating with ethylene oxide gas), gamma irradiation, or ethanol (e.g., 70% ethanol).

In certain embodiments, the methods of the instant invention further comprise coating the flocked substrate with additional materials to enhance its properties. For example, the flocked substrate may be coated with collagen, a proteoglycans, elastin, or a glycosaminoglycans (e.g., hyaluronic acid, heparin, chondroitin sulfate, or keratan sulfate). In a particular embodiment, the flocked substrate comprises a material that enhances the flocked substrate's ability to absorb fluids, particularly aqueous solutions (e.g., blood). In a particular embodiment, the flocked substrate is coated with the material which enhances the absorption properties. The term “coat” refers to a layer of a substance/material on the surface of a structure. Coatings may, but need not, also impregnate the flocked substrate. Further, while a coating may cover 100% of the flocked substrate, a coating may also cover less than 100% of the surface of the flocked substrate (e.g., at least about 75%, at least about 80%, at least about 85%, at least about 90%, at least about 95%, at least about 98%, or more of the surface may be coated). Materials which enhance the absorption properties of the flocked substrate include, without limitation: gelatin, alginate, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In a particular embodiment, the material is a hydrogel (e.g., a polymer matrix able to retain water, particularly large amounts of water, in a swollen state). In a particular embodiment, the material is gelatin. In a particular embodiment, the flocked substrate is coated with about 0.05% to about 10% coating material (e g., gelatin), particularly about 0.1% to about 10% coating material (e.g., gelatin) or about 0.1% to about 1% coating material (e.g., gelatin). In a particular embodiment, the material (e.g., hydrogel) is crosslinked.

In certain embodiments, the methods of the instant invention further comprise mineralization of the flocked substrate. In certain embodiments, the flocked substrate comprises minerals and/or is coated with minerals. Mineralization, for example, with hydroxyapatite, can enhance the adhesion of osteogenic precursor cells in vitro and in vivo (Duan, et al., Biomacromolecules (2017) 18:2080-2089). In a particular embodiment, the flocked substrate is coated with Ca, P, and O. In a particular embodiment, the flocked substrate is coated with hydroxyapatite, fluorapatite, or chlorapatite, particularly hydroxyapatite. In a particular embodiment, the flocked substrate is immersed in simulated body fluid (SBF) for the mineralization (e.g., a solution comprising NaCl, CaCl₂, NaH₂PO₄, and NaHCO₃).

In certain embodiments, the flocked substrate of the instant invention may comprise and/or encapsulate cells and/or tissue. For example, the methods of the instant invention may comprise seeding cells and/or tissue on the flocked substrate. In certain embodiments, the cells are autologous to the subject to be treated with the flocked substrate. The flocked substrate may comprise and/or encapsulate any cell type. Cell types include, without limitation: embryonic stem cells, adult stem cells, bone marrow stem cells, induced pluripotent stem cells, progenitor cells (e.g., neural progenitor cells), embryonic like stem cells, mesenchymal stem cells, CAR-T cells, immune cells (including but not limited to T cells, B cells, NK cells, macrophages, neutrophils, dendritic cells and modified forms of these cells and various combinations thereof), cell based vaccines, and cell lines expressing desired therapeutic proteins and/or genes. In a particular embodiment, the cells comprise stem cells. In a particular embodiment, the cells comprise dermal fibroblasts. In a particular embodiment, the flocked substrate comprises and/or encapsulates cell spheroids. In a particular embodiment, the flocked substrate comprises and/or encapsulates tissue samples (e.g., minced tissue), such as skin tissue samples or bone samples. The cells or tissue may be cultured within the flocked substrate (e.g., the cells or tissue may be cultured for sufficient time to allow for growth within and/or infiltration into the flocked substrate). For example, the cells or tissue may be cultured in the flocked substrate for 1 day, 2 days, 3 days, 4 days, 5 days, or more.

The flocked substrate of the instant invention may comprise or encapsulate at least one agent, particularly a bioactive agent, biologic, and/or drug. The agent may be added to the flocked substrate during synthesis and/or after synthesis. The agent may be conjugated (e.g., directly or via a linker) to the flocked substrate and/or coating material, encapsulated by the flocked substrate, and/or coated on the flocked substrate (e.g., with, underneath, and/or on top of the coating that enhances the flocked substrate's ability to absorb fluids, if present). In a particular embodiment, the agent is not directly conjugated to the flocked substrate. In a particular embodiment, the agent is conjugated or linked to the flocked substrate (e.g., surface conjugation or coating). In a particular embodiment, the agents are administered with but not incorporated into the flocked substrate.

Biologics include but are not limited to proteins, peptides, antibodies, antibody fragments, DNA, RNA, and other known biologic substances, particularly those that have therapeutic use. In a particular embodiment, the agent is a drug or therapeutic agent (e.g., a small molecule) (e.g., analgesic, growth factor, anti-inflammatory, signaling molecule, cytokine, antimicrobial (e.g., antibacterial, antibiotic, antiviral, and/or antifungal), blood clotting agent, factor, or protein, pain medications (e.g., anesthetics), etc.). In a particular embodiment, the agent enhances tissue regeneration, tissue growth, and wound healing (e.g., growth factors). In a particular embodiment, the agent treats/prevents infections (e.g., antimicrobials such as antibacterials, antivirals and/or antifungals). In a particular embodiment, the agent is an antimicrobial, particularly an antibacterial. In a particular embodiment, the agent enhances wound healing and/or enhances tissue regeneration (e.g., bone, tendon, cartilage, skin, nerve, and/or blood vessel). Such agents include, for example, growth factors, cytokines, chemokines, immunomodulating compounds, and small molecules. Growth factors include, without limitation: platelet derived growth factors (PDGF), vascular endothelial growth factors (VEGF), epidermal growth factors (EGF), fibroblast growth factors (FGF; e.g., basic fibroblast growth factor (bFGF)), insulin-like growth factors (IGF-1 and/or IGF-2), bone morphogenetic proteins (e.g., BMP-2, BMP-7, BMP-12, BMP-9; particularly BMP-2 fragments, peptides, and/or analogs thereof), transforming growth factors (e.g., TGFβ, TGFβ3), nerve growth factors (NGF), neurotrophic factors, stromal derived factor-1 (SDF-1), granulocyte-macrophage colony-stimulating factor (GM-CSF), granulocyte-colony stimulating factor (G-CSF), erythropoietin (EPO), glial cell-derived neurotrophic factors (GDNF), hepatocyte growth factors (HGF), keratinocyte growth factors (KGF), and/or growth factor mimicking peptides (e.g., VEGF mimicking peptides). Chemokines include, without limitation: CCL21, CCL22, CCL2, CCL3, CCL5, CCL7, CCL8, CCL13, CCL17, CXCL9, CXCL10, and CXCL11. Cytokines include without limitation IL-2 subfamily cytokines, interferon subfamily cytokines, IL-10 subfamily cytokines, IL-1, I-18, IL-17, tumor necrosis factor, and transforming-growth factor beta superfamily cytokines. Examples of small molecule drugs/therapeutic agents include, without limitation, simvastatin, kartogenin, retinoic acid, paclitaxel, vitamins (e.g., vitamin D3), etc. In a particular embodiment, the agent is a blood clotting factor such as thrombin or fibrinogen. In a particular embodiment, the agent is a bone morphogenetic protein (e.g., BMP-2, BMP-7, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 1). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 1). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

Flocked substrates as described herein are also encompassed by the instant invention. In certain embodiments, the flocked substrates are synthesized by the methods described herein. Compositions comprising the flocked substrate and at least one carrier (e.g., pharmaceutically acceptable carrier) are also encompassed by the instant invention.

The flocked substrates of the instant invention can be used for many purposes including, without limitation: regenerative medicine, wound healing, hernia repair, and sample collection. The perpendicular alignment of the microfibers allows for thousands or more of tissue adhesion points, serving to establish a structurally sound tissue layer or anchor points around whatever object is flocked, particularly tissue-to-biomaterial interfaces. In certain embodiments, the flocked substrates are used as a tissue scaffold or 3D cell culture scaffolds. In certain embodiments, the flocked structure is used as a scaffold for cellular adhesion and growth for treating or repairing tissue damage.

The flocked structures of the instant invention can be used to create tissue architectures for a variety of application including, without limitation: wound healing, tissue engineering, tissue growth, tissue repair, bone repair, tissue regeneration, and engineering 3D in vitro tissue models. The flocked structures can also be combined with a variety of hydrogels or biological matrices/cues to form 3D hybrid scaffolds that can release biologically functional agents. The tissue constructs can be used for regeneration of many tissue defects (e.g., skin, bone) and healing of various wounds (e.g., injuries, diabetic wounds, venous ulcer, pressure ulcer, burns). The flocked structures may be used ex vivo to generate tissue or tissue constructs/models. The flocked structures may also be used in vivo in patients (e.g., human or animal) for the treatment of various diseases, disorders, and wounds. In a particular embodiment, the flocked structure stimulates the growth of existing tissue and/or repair of a wound or defect when applied in vivo. The flocked structures can be used for engineering, growing, and/or regeneration of a variety of tissues including but not limited to skin, bone, cartilage, muscle, nervous tissue, and organs (or portions thereof).

In accordance with the instant invention, the flocked structures may be used in inducing and/or improving/enhancing wound healing and inducing and/or improving/enhancing tissue regeneration. The flocked structures of the present invention can be used for the treatment, inhibition, and/or prevention of any injury or wound. In a particular embodiment, the method comprises administering a flocked structure, optionally comprising an agent and/or cell as described herein. For example, the flocked structure can be applied to a wound with the flocked microfibers facing inward to the wound and the substrate towards the surface. In certain embodiments, the flocked structure can be applied to a wound (e.g., a bone (e.g., cranial) deficiency) with the flocked microfibers facing outward (optionally wherein the substrate is in line or even with the bone). Flocked structure of the instant invention can be loaded with different cell types as necessary for regeneration of various tissues (e.g., bone). In a particular embodiment, the flocked structure comprises blood clotting factors (e.g., for accelerating blood clot formation and/or preventing blood loss). For example, the flocked structure can be used to induce, improve, or enhance wound healing associated with surgery (including non-elective (e.g., emergency) surgical procedures or elective surgical procedures). Elective surgical procedures include, without limitation: liver resection, partial nephrectomy, cholecystectomy, vascular suture line reinforcement and neurosurgical procedures. Non-elective surgical procedures include, without limitation: severe epistaxis, splenic injury, liver fracture, cavitary wounds, minor cuts, punctures, gunshot wounds, and shrapnel wounds. The flocked structures may be delivered directly into a cavity (such as the peritoneal cavity), optionally using a pressurized cannula.

In accordance with the instant invention, the flocked structures of the present invention can be used to treat and/or prevent a variety of diseases and disorders. Examples of diseases and/or disorders include but are not limited to wounds, ulcers, infections, hemorrhage, tissue injury, tissue defects, tissue damage, bone fractures, bone degeneration, cancer (e.g., the use of docetaxel and curcumin for the treatment of colorectal cancer (Fan, et al., Sci. Rep. (2016) 6:28373)), neurologic diseases (e.g., Alzheimer's and Parkinson's), ischemic diseases, inflammatory diseases and disorders, heart disease, myocardial infarction, and stroke. Methods for inducing and/or improving/enhancing wound healing in a subject are also encompassed by the instant invention. Methods of inducing and/or improving/enhancing tissue regeneration (e.g., blood vessel growth, neural tissue regeneration, and bone regeneration) in a subject are also encompassed by the instant invention. Methods of inducing and/or improving/enhancing hemostasis in a subject are also encompassed by the instant invention. The methods of the instant invention comprise administering or applying a flocked structure of the instant invention to the subject (e.g., at or in a wound or bone defect or hole (e.g., cranial defect)). In a particular embodiment, the method comprises administering a flocked structure comprising an agent and/or cell as described herein. Flocked structures of the instant invention can be loaded with different cell types as necessary for regeneration of various tissues. In a particular embodiment, the flocked structure comprises blood clotting factors (e.g., for accelerating blood clot formation and/or preventing blood loss). In a particular embodiment, the method comprises administering a flocked structure to the subject and an agent as described herein (i.e., the agent is not contained within the flocked structure). When administered separately, the flocked structure may be administered simultaneously and/or sequentially with the agent. The methods may comprise the administration of one or more flocked structure. When more than one flocked structure is administered, the flocked structures may be administered simultaneously and/or sequentially.

The flocked structures can also be used to expand and increase cell numbers (e.g., stem cell numbers) in culture. In a particular embodiment, microtissues can be grown in situ by prolonged culture of cell laden flocked structure (e.g., in confined microfluidic channel devices). These microtissues are injectable or transplantable into a tissue defect to promote wound healing in a subject (e.g., the flocked structures comprise autologous cells).

The flocked structure may also be employed for cell detection, separation, and/or isolation of cell populations in a mixture. For example, flocked structures conjugated to specific antibodies can be used for the isolation, separation, and/or expansion of different cell types from their mixtures (Custodio, et al., Biomaterials (2015) 43:23-31). Further, flocked structures can be used for the in vitro adhesion, proliferation, and/or maturation of chondrocytes as well as in vivo cartilage formation and osteochondral repair induced by flocked structure when administered together with chondrocytes (Liu, et al., Nat. Mater. (2011) 10:398-406).

The flocked structures of the present invention may be administered by any method. The flocked structures may be administered to a subject or a patient as a pharmaceutical composition. The compositions of the instant invention comprise a flocked structure and a pharmaceutically acceptable carrier. The term “patient” as used herein refers to human or animal subjects. These compositions may be employed therapeutically, under the guidance of a physician.

The compositions of the instant invention may be conveniently formulated for administration with any pharmaceutically acceptable carrier(s). For example, the agents may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Except insofar as any conventional media or agent is incompatible with the agents to be administered, its use in the pharmaceutical preparation is contemplated.

The flocked structures of the instant invention may be administered by any method. For example, the flocked structure of the instant invention can be administered, without limitation, by implantation (e.g., surgical implantation) or application to the desired site.

In a particular embodiment of the instant invention, methods for modulating (increasing) hemostasis; inhibiting blood loss; and/or treating hemorrhage are provided. In a particular embodiment, the method comprises administering a flocked substrate to the wound or site of bleeding. In a particular embodiment, the flocked structures comprise a blood clotting factor such as thrombin and/or fibrinogen.

In a particular embodiment of the instant invention, methods for stimulating bone regeneration and/or treating bone loss are provided. In a particular embodiment of the instant invention, methods for stimulating chondral, osteochondral, and/or cartilage regeneration and/or treating chondral, osteochondral, and/or cartilage loss are provided. In a particular embodiment, the method comprises administering a flocked structure to the site of bone loss (or chondral, osteochondral, and/or cartilage loss). In a particular embodiment, the site of bone loss is periodontal. In a particular embodiment, the flocked structure is mineralized. In a particular embodiment, the flocked structure comprises a bone growth stimulating growth factor such as a bone morphogenic protein or fragment or analog thereof. In a particular embodiment, the agent is a bone morphogenetic protein (e.g., BMP-2, BMP-7, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 1). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 1). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37ºC, particularly below about 34ºC, may be considered hydrophobic.

As used herein, the term “hydrophilic” means the ability to dissolve in water. In a particular embodiment, polymers that at 1% solution in bi-distilled water have a cloud point above about 37ºC, particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “conductive” or “electrically conductive” mean a material which is capable of electrical conductivity without the addition of other elements. As used herein, the term “insulative” refers to a type of material through which virtually no or very little current flows or the capability of the material to prevent or inhibit the flow of electricity or electrons.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopeptides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, triamcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following examples illustrate certain embodiments of the invention. They are not intended to limit the invention in any way.

### Example 1

### Materials and Methods

### Wet Spinning Continuous Conductive Microfibers

To fabricate continuous conductive microfiber tow, PCL pellets and AgNP powder (70 nm) were dissolved in dichloromethane (DCM):dimethylformamide (DMF) (4:1) to yield a 20% w/v PCL and 0-0.75% w/v AgNP polymeric solution. The PCL and AgNP solution was allowed to dissolve completely and was subsequently mixed thoroughly under gentle heat (65° C.) and stirring. Once the solution was homogenous, it was loaded into a syringe and extruded through a 3D printed 5-emitter extrusion device with 21 ga needles on a benchtop wet spinning device at a rate of 3.0 mL h⁻¹ into a coagulation bath of 70% room temperature ethanol. The multiple emitter device was designed with five outlets and one inlet and was printed using a digital light processing 3D printer (Vida, EnvisionTEC) and Clear Guide material (EnvisionTEC). The base of the emitter is 63 mm long, 8 mm wide, and 8 mm tall, with outlet fittings extruding 7 mm from the base. The inlet is on the end of the base, perpendicular to the five outlet nozzles. The outlets nozzles are arranged in a linear array and are spaced 12 mm from their centers. Fibers were collected by a drum collector ≈6 in. above the coagulation bath. After the wet spinning process was finished, the fiber tow was allowed to dry completely and was subsequently removed by razor and manually stretched to align the polymer molecules.

Producing Flocking Fibers from Fiber Tow

In order to convert the fiber tow to flocking fibers, a two-step process was developed. First, the fiber tow was spread out fanwise and manually cut at ≈1 mm in length and collected in a collecting bowl or by vacuum. Next, since fiber fusion along cutting lines is inevitable due to PCLs thermoplastic nature, fibers had to be mechanically separated. All collected fibers were suspended in a 500 ml beaker of ice water and sonicated in successive 1 hour intervals until the majority of fibers were separated and appeared as a slurry. Any large remaining fiber clumps were further broken up by vigorous bubbling using an adapted bubbler in a 1 L Erlenmeyer flask. The resulting fiber slurry was strained with a mesh strainer and allowed to dry under vacuum. All fibers were stored in humid (70% conditions until ready for use).

### Fabrication of Adhesives

Both CHS and gel adhesives were prepared as reported. Briefly gel adhesives were prepared by dissolving 2 g of gel in 10 mL of H₂O at 50° C. under constant stirring for 24 hours, yielding a 20% w/v gel solution (Lewis, et al. (2018) Text. Res. J., 88:392). CHS adhesive was prepared by dissolving 0.5 g of CHS 95/500 in 10 mL of a 5% w/w AcOH/H₂O solution and stirring at 50° C. for 24 hours, yielding a 5% w/v CHS adhesive solution (Tu, et al. (2019) Small, 15:1902070). Additionally, a 1:1 gel:CHS blend was prepared by mixing both CHS and gel solutions under gentle heating and stirring. Before flocking, each adhesive was warmed to 50° C. and poured onto the substrate or electrode. A razor blade was used to knife coat the entire surface of the substrate. 3D printed scaffolds were dip-coated in a 50 mL beaker of adhesive and allowed to drip until only a fine coating remained.

### Fabrication of Substrates

Pure CHS substrates were prepared based on a reported method (Tu, et al. (2019) Small 15:1902070; Hitzbleck, et al. (2013) Adv. Mater., 25:2672). Briefly, a 20% CHS solution was poured onto an electrode and dried at 60° C. overnight. Additionally, 3D circular mesh substrates were prepared by 3D-printing a similar material as reported (Aldrich, et al. (2019) ACS Appl. Mater. Interfaces, 11:12298; Zhang, et al. (2020) Biofabrication 12:035020). Briefly, mesh scaffolds were extrusion 3D-printed using a BioPlotter (EnvisionTEC) using a 22 gauge needle. Meshes are composed of 35% w/v PCL (MW=80000, Sigma-Aldrich, St. Louis, MO) and 1:2 hydroxyapatite (Hap nanocrystals, avg. 100 nm, Berkeley Advanced Biomaterials, Inc., Berkeley, CA) in 1:1:1 DCM:DMF:tetrahydrofuran. The meshes were printed with an 8 mm diameter outside border with infill strands 1 mm apart and consist of three layers measuring ≈1.2 mm thick. The layers are stacked with strands perpendicular to the preceding layer and create a porous structure. The scaffolds were lyophilized for 24 hours and subsequently plasma treated before use. Electrospun membranes were synthesized as reported (Chen, et al. (2018) J. Mater. Chem. B, 6:393). Briefly, a 10% w/v solution of pure PCL in 4:1 DCM:DMF was electrospun at a flow rate of 0.8 mL h⁻¹, voltage of 14 kV, RH of 40-65%, and collected on a rotation collector until the desired thickness was achieved. Due to the nature of the CHS on the 3D printed and CHS substrates, no additional adhesive was used to fix the substrates to the grounded electrode. For the PCL nanofiber mats, squares slightly larger than the electrode were cut and the corners taped around the electrode.

### Flocked Scaffold Assembly

Prior to flocking, flock fibers were removed from humidity-controlled storage and loaded into a mechanical sieve attached to a motor (MaagFlock, Germany). The fibers were sieved onto the surface of the electrode until the surface of the electrode was completely covered and fibers were uniformly distributed. Next, the ground electrode was affixed roughly 4 inches from the charging electrode and a voltage was applied in sweeping cycles. Voltage gradually increased from 30 to 60 kV in cycles to avoid cloud formation. Once the fibers were adequately flocked, the electrode was removed and dried in a 60° C. oven for 5 minutes and allowed to thermally cure at ambient conditions for 1-2 hours. After curing, scaffolds were removed using a razor blade. Scaffolds were then crosslinked in a 1% glutaraldehyde chamber for 24 hours. Finally, finished scaffolds were washed with deionized H₂O five times to remove any loose fibers and stored at ambient conditions.

### Analysis of Flock Time on Scaffold Structure

Analysis of flocking time was determined by calculating the number of fibers flocked as a function of time. An equivalent (10 g) load of flock fibers were evenly sieved onto the charging electrode and a uniform voltage swinging from 30 to 60 kV was applied for 30 seconds, 1 minute, 5 minutes, 10 minutes, and 15 minutes. Scaffolds prepared at each time point were imaged by SEM and the fiber density was calculated by taking the average fiber count of four representative areas of equivalent size. Fiber counting was carried out using a modified method for counting cells on ImageJ, where the tops of fibers were counted as circles.

### Quantification of Porosity

Scaffold porosity was determined using a reported method. Briefly, the volume for total scaffold was derived by assuming a solid cylinder with a diameter (D) the size of the substrate and height (h) as the length of the fibers. Next, the total fiber volume was subtracted from the total scaffold volume, leaving porous volume remaining.

### Fiber Flockability Determination

To determine the quality of flockability, fibers of different compositions and finishes were prepared. After drying, roughly 1 g of each fiber type of sieved and flocked with a swinging voltage from 30 to 60 kV. After flocking for 1 minute, the mass of the remaining fibers was taken. The total fibers lifted were determined as the difference between sieved fiber mass and remaining fiber mass. Similarly, to determine the effect of humidity on flockability, fibers were incubated at 0%, 10%, 20%, 40%, and 50% RH for 1 hour before flocking in ambient conditions.

### Characterization of Flocking Fibers

Fiber morphology was characterized using a scanning electron microscope (SEI, Germany) and USB microscope at different steps in the flock fiber fabrication process. To quantify diameter and length of fibers, SEM images were analyzed using ImageJ length analysis. To quantify the mechanical properties of the fibers, an ultimate tensile strength test was used (Agilent, Keysight). To determine fiber conductivities, an equation from the percolation theory was derived and served to estimate conductivities as a function of AgNP concentration. ICP-MS was used to determine the AgNP concentration within bulk fibers as reported. Briefly, 10 g of fibers were dissolved in DCM and analyzed by ICP-MS. Similarly, the amount of Ag+ eluted from fibers was determined by submerging 10 g of fibers in 300 mL of PBS at 37° C. for 1, 4, 7, 14, and 28 days. At each time point, the PBS was mixed and removed from each well, stored in a 50 mL jar, frozen, and lyophilized. Following freeze-drying, samples were analyzed by ICP-MS using the aforementioned methodology.

### Characterization of Flocked Scaffolds

Scaffolds were mechanically characterized by an abrasion test and by a tearing test. For abrasion resistance analysis, scaffolds were fixed to the bottom of a bowl of deionized H₂O with double sided tape. Pressure was applied using the tip of the thumb and a back-and-forth rubbing was carried out under wet conditions. Abrasion resistance was summarized by the change in mass as a function of rubbing cycles. Scaffold masses were taken after 5, 10, 15, 20 25, 35, 45, 60, 80, and 100 cycles. Critical fracture energy was measured using a trouser tear test, where the substrate was subject to iterative uniaxial tears on a mechanical testing machine (UTM Keysight). All scaffolds were imaged by SEM and camera photographs. Distribution of fiber orientation (quality of alignment or degree of anisotropy) was analyzed using OrientationJ, an open source plugin to use with ImageJ FIJI. Scaffold degradation was measured by submerging scaffolds in a reported BSF for 1, 3, 5, 7, 10, 15, 20, 25, 30, and 35 days and recording the change in mass of each scaffold.

### Fabrication of Thermoresponsive Self-Folding Flocked Biphasic Nanofiber Tube

Nanofiber mats were electrospun as reported using 10% w/v PCL. Briefly, 10% w/v PCL was dissolved in solvent mixture of 4 parts DCM and 1 part DMF (4:1 DCM:DMF) and the resulting polymer solution was electrospun at 12 kV at 0.8 mL h⁻¹ and collected on a rotating mandrel. The resulting PCL mats were plasma treated with atmospheric oxygen for 3 minutes, submerged in water, and a thin layer of CHS adhesive (5% w/v CHS in AcOH) was knife-coated on the surface. Next, the nanofiber mat was fixed to the ground electrode by pushing the wet mat firmly against the surface. Approximately 0.5 g of FeCl₃ PLA flock fibers (supplied by Spectro Coating) were mechanically sieved onto the charging electrode and flocked onto the surface of the nanofiber mat by an applied voltage gradually increasing from 30 to 60 kV. The flocked mat was removed and placed a 45° C. oven for 30 minute to initiate self-folding. After heating, the flocked mat was placed in a desiccator overnight. A small portion of overlapping nanofiber mat was treated with CHS adhesive (gluing the overlapping edges of the tube) and crosslinked in a GA chamber for 24 hours the following day. Before imaging, the tubes were cut into smaller thoroughly washed with deionized water to remove any loose fibers.

### Cell Proliferation Assay

After preparing AgNP/PCL scaffolds, an 8 mm biopsy punch was used to create circular scaffolds that would fit inside a 48 well plate. After punching scaffolds to size, each scaffold was sterilized by ethanol submersion for 24 hours and UV sterilization for 12 hours. Prior to cell seeding, all scaffolds were rinsed five times with PBS and time times with cell culture media. Prior to moving each scaffold into the 48 well plate, each well bottom was coated with 1% agarose to prevent cell adhesion to the plate bottom. Rat bone marrow derived stem cells (rBMSCs) were seeded as reported. Briefly, 300 μL of cell suspension of 30 000 cells mL⁻¹ was added dropwise directly onto the flocked scaffold and cultured in complete DMEM cell media at 37° C. After 24 hours, the media was replaced, and was replaced every 48 hours for the remainder of the experiment. At 1, 3, 5, and 7 days, cells were stained with LIVE/DEAD stain and imaged using confocal microscopy. Additionally, at each time point, cell counts were taken using a cell counter.

### Cell Viability

To assess the cytotoxicity of the AgNP/PCL scaffold with differing amounts of AgNP, PCL fibers were prepared with 0%, 0.01%, 0.25%, 0.5%, and 0.75% AgNP. Fibers were prepared, cut, separated, and sterilized as mentioned. However, since fibers with low AgNP concentrations cannot flock, fibers were used to make a small layer on the bottom of each well in a 48 well plate. Exactly 300 μL of a 30 000 cells mL⁻¹ rBMSC cell suspension was added directly to 1% agarose-covered well of a 48 well plate. Media was changed after the first 24 hours, and every 48 hours following seeding. After 1, 3, and 5 days, scaffolds with cells were removed and added to a new plate with fresh media and CCK-8 solution and incubated for 4 hours. After incubation, absorbance values were measured at 450 nm wavelength on a microplate reader (GenTek).

### 3D Tissue Culture

To observe the spatial distribution of cells on flock fibers, rBMSCs were cultured on 0.5% AgNP/PCL scaffolds using the described steps. Cells were seeded at 150 000 cells mL⁻¹ and cultured for 28 days. At 1, 14, and 28 days, scaffolds were removed and stained with actin and DAPI stains. Stained scaffolds were imaged using confocal microscopy. To compile 3D images, z-stack images were taken at 10 μm intervals over a total length of 1 mm and a 3D image was generated using Zeiss 3D image viewer. The average depth of cell penetration was generated by measuring the distance of 20 randomly selected cells from the substrate.

### Antibacterial Study

To assess the potential antibacterial efficacy of the AgNP/PCL flocked scaffolds, two antibacterial assays were carried out as reported. First, a zone of inhibition contact study was carried out with 0.5% AgNP/PCL. Briefly, 100 μL Methicillin-resistant S. aureus USA 300 (MRSA, ≈10⁷ CFU mL⁻¹) suspension solution was cultured on an LB-agar coated petri dish. Flocked scaffolds were directly placed onto the surface of the bacterial culture and incubated at 37° C. overnight. The following day, the zone of inhibition was recorded via photography. After ascertaining an antibacterial effect, another antibacterial culture study was carried out by directly adding each scaffold component and fibers with 0%, 0.01%, 0.25%, 0.5%, and 0.75% AgNP directly to the ≈10⁷ CFU mL⁻¹ MRSA suspension and incubating overnight. The following bacterial culture was plated directly to an LB-agar coated petri dish and incubated overnight again. The resulting CFUs were counted at each fiber composition using the open source ImageJ FIJI plugin, ColonyCounter.

### Subcutaneous Implantation

12 male Sprague-Dawley rats (8-10 weeks old) were purchased from Charles Rivers Laboratories and housed in an AAALAC-accredited animal facility at 20.0-26.1° C. with a set point of 22.2° C., 30-70% humidity with a set point of ≈40%, and light cycles (12 hours on and 12 hours off). Animals were monitored continuously by Comparative Medicine via the Edstrom Watchdog system at University of Nebraska Medical Center (UNMC) and were pair-housed in each cage with unrestricted access to water and food (#8656 Sterilizable 4%, Envigo). Animals were allowed 3 days for acclimation facilities before the study began. The animals were randomly divided into 3 groups of 4 (Group “low flock”, Group “medium flock”, Group “high flock”). Carprofen (5 mg kg⁻¹) was administered once subcutaneously prior to surgery and the rats were induced by using 4% isoflurane in oxygen for ≈2 minutes and maintained by 2% isoflurane during the procedure. Rats were placed on a circulating warm blanket to maintain their body temperature. An area of 8×4 cm² on the back of each animal was shaved, and povidone-iodine solution and 70% ethanol were applied three times on exposed skin. Flocked scaffolds were sterilized by ethylene oxide for 24 hours one day before implantation and rinsed with saline before implanting. Each rat received 4 of the same type of flocked scaffold, with the fibers facing away from the underlying muscle. Each treatment group had 4 rats. There were 16 implants for each group (8 implants/group/each designed ending time point). Subcutaneous pockets were made through 1.5 cm incisions at 4 supraspinal sites on the dorsum. Each sample was inserted into a subcutaneous pocket by a tweezer, and then the skin incisions were closed with a stapler. Rats were euthanized by CO₂ at 1 or 2 weeks post-implantation. Each explant with surrounding tissue was gently dissected out of its subcutaneous pocket, and then immersed in formalin for at least 3 days prior to histology analysis.

### Statistics and Graphics

All data were expressed as mean±standard deviation and a minimum sample size of 3 was used in each experiment. For pairwise columnar comparisons, ordinary one-way ANOVAs with Tukey's multiple comparisons post hoc testing were performed. Similarly, for grouped pairwise comparisons, 2-way ANOVAs with Tukey's multiple comparisons post hoc testing was performed. Estimated conductivities using the Percolation Theory were expressed as single points based on theoretical data following the equation described in this work. Fiber alignment was interpolated from single points to a continuous line using nonlinear fitting. Normalized (adjusted curves to fall from 0° to 180°) and smoothed distribution curves were compared using a Kolmogorov-Smirnov test. Flock yields were expressed as heat maps, with each box expressing the average of 3 sample values. The equation for AgNP filler fraction (y=36.53x+0.2849) was generated using a simple linear regression. All measurements on photographs/SEM microphotographs were taken using ImageJ after calibrating pixels to mm/μm. Statistical significance was expressed as *p<0.05, ** p<0.01, *** p<0.001, **** p<0.0001, and a lack of pairwise comparison bars implies a lack of significance (p>0.05). Figures are all original and were created using BioRender and Microsoft PowerPoint. All statistical analyses were performed using GraphPad Prism 9 for macOS (version 9.1.1).

### Results

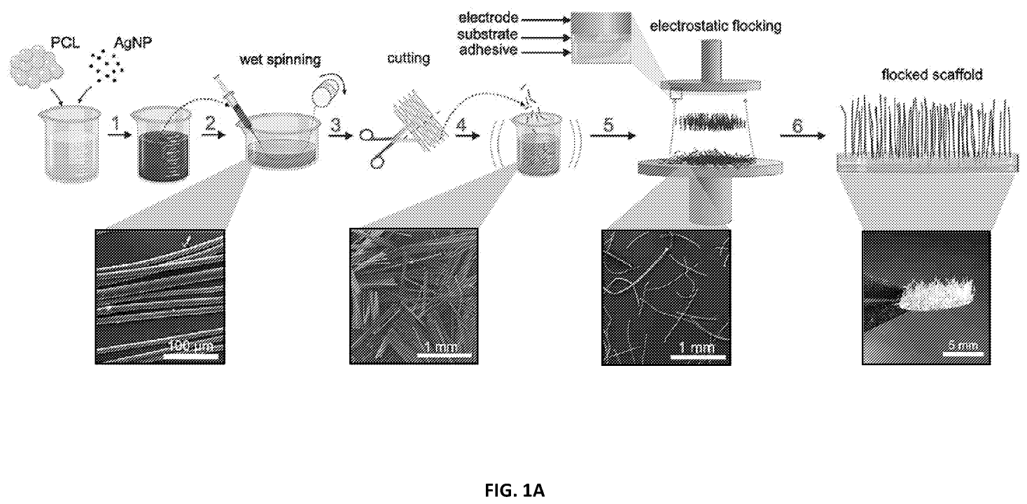

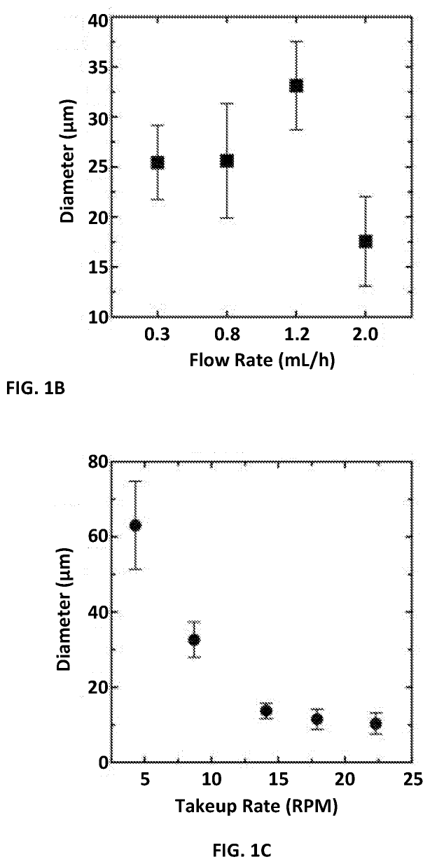

Flocked scaffold fabrication comprised three main steps: 1) wet spinning, 2) cutting and separating wet-spun fibers, and 3) electrostatic flocking (FIG. 1). First, a designated polymer and conductive filler (e.g., PCL and AgNPs) were blended in organic solvents until homogenous. Next, the mixed solution was wet-spun using a multinozzle wet spinning device and collected on a barrel collector. Briefly, a multi-emitter wet spinning set up utilizing a 5-emitter 3D-printed extrusion head, syringe pump, 24×4.5 in coagulation basin, and barrel collector was used. Optimization of parameters (e.g., ejection rate and take-up rate) during wet spinning provided a framework for consistent synthesis of microfibers with controllable diameters ranging from ≈10 to 65 μm (FIGS. 1B and 1C). Regulating flock fiber diameter is an important consideration when developing flocked scaffolds because fiber diameter significantly influences fiber motility and orientation (Lee, et al. (2018) J. Korean Phys. Soc., 73:152). Following the wet spinning procedure, fibers were mechanically cut and then separated by ultrasonic probe sonication, followed by bubbling. After separation and drying, fibers were mechanically sieved onto a charging electrode, with either an adhesive-covered substrate affixed to the ground electrode or an adhesive layer (acting as both adhesive and substrate) directly coated on the ground electrode. Fiber deformity may be common, so sieving or sifting of cut fibers ensures only uniform fibers are flocked. Adhesives varied from those reported (20% w/v gelatin (gel), 5% w/v CHS in acetic acid (AcOH)+thermal crosslinking) to a 1:1 blend of 20% w/v gel: 5% w/v CHS in AcOH, which was used for all in vitro and in vivo tests (Gossla, et al. (2016) Acta Biomater., 44:267; Steck, et al. (2010) Tissue Eng., 16:3697; Balasubramanian, et al. (2015) Mater. Lett., 158:313). Briefly, chitin can be deacetylated into chitosan and dissolved in acetic acid (5% w/v), poured onto the ground electrode, and thermally cured and chemically crosslinked with glutaraldehyde. A gradually increasing voltage was applied to the charging electrode (0-60 kV) for a specified time (½, 1, 5, 10, or 15 minutes) or until all of the flocking fibers lifted. Gradually increasing the applied voltage ensured that fibers would flock at a moderate, continuous rate and avoid interference while in flight. Finished scaffolds were dried completely and chemically crosslinked in a glutaraldehyde (GA) chamber for 24 hours. Resulting scaffolds had velvety appearances with visibly aligned fibers (FIG. 1A (bottom right)).

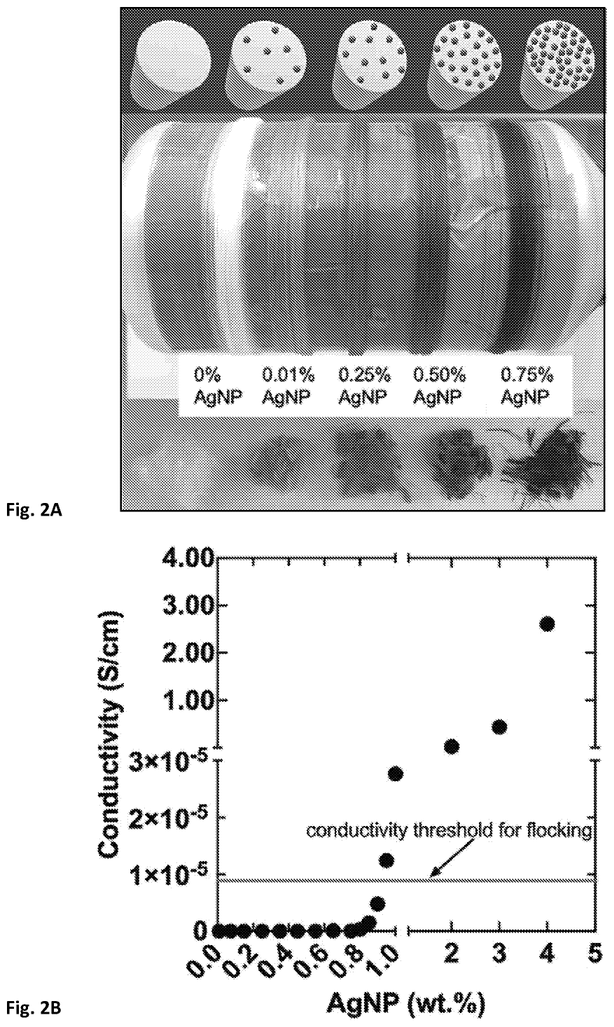

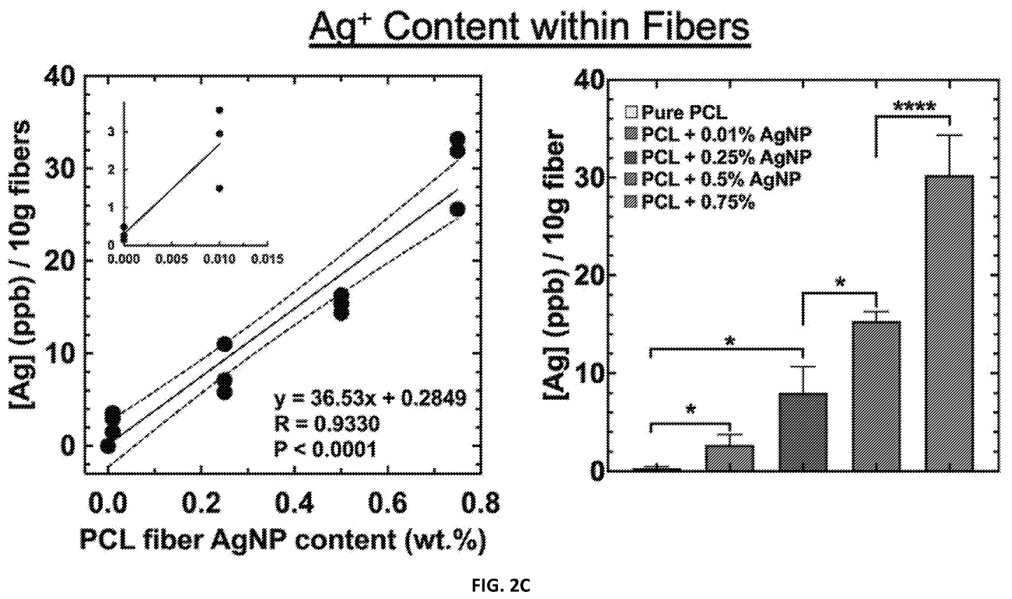

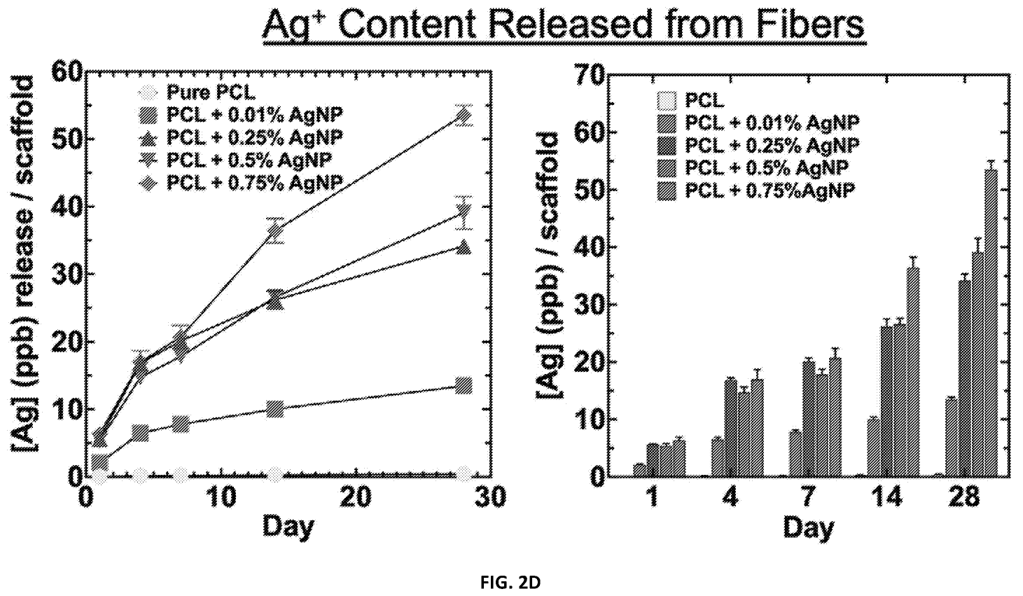

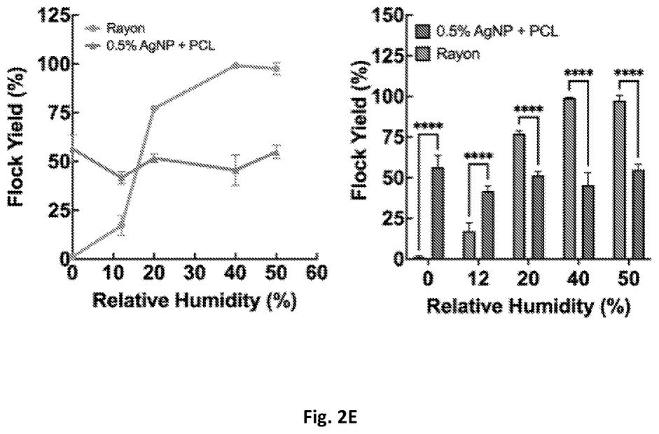

An important step in the fabrication of flocked scaffolds with insulative polymer fibers is encapsulating an adequate filler volume to achieve sufficient charging for flocking. In most cases, flock fibers have a surface finish applied, allowing for the accumulation of charge along their surface, with flock yield determined by the respective surface moisture content. CHS may have proved to be sufficiently conductive to accumulate charge. However, for many materials, such as insulative polymer fibers, sufficient charge accumulation is difficult or impossible. By incorporating conductive fillers in the polymer matrix, charge accumulation throughout the bulk of the fiber is made possible. To demonstrate how this flocking mechanism is fundamentally different than each of those reported (utilizing fiber finishes or inherently semiconductive materials), 0.5% AgNP/PCL fibers and surface-finished Rayon fibers were flocked after incubating at a different relative humidity (RH) for 24 hours (FIG. 2E) (Vellayappan, et al. (2015) RSC Adv., 5:73225; Gokarneshan, N. (2018) Curr Trends Fashion Technol Textile Eng., 4:555630; Phillippi, et al. (2001) Aquaculture 195:225; Xu, et al. (2021) J. Colloid Interface Sci., 588:9; Guo, et al. (2021) Adv. Sustainable Syst., 5:2000202; Tu, et al. (2019) Small 15:1902070; Hitzbleck, et al. (2013) Adv. Mater., 25:2672; Uetani, et al. (2014) Adv. Mater., 26:5857; Gossla, et al. (2016) Acta Biomater., 44:267; Steck, et al. (2010) Tissue Eng., 16:3697; Walther, et al. (2012) Materials 5:540; Walther, et al. (2007) Text. Res. J., 77:892; Tonndorf, et al. (2018) Text. Res. J., 88:1965; Balasubramanian, et al. (2015) Mater. Lett., 158:313). Rayon fibers exhibited a sigmoidal response to RH, while the 0.5% AgNP/PCL fibers were largely unaffected by RH. As surface-finishes increase the moisture content of Rayon fibers, their performance is largely dictated by the humidity at which they are flocked and the subsequent moisture content on the fiber surface. The humidity-independent flockability of the 0.5% AgNP/PCL fibers demonstrates a different method for surface charge accumulation. To further investigate the effects of percolation charging on flocking, PCL fibers were created with 0.00%, 0.01%, 0.25%, 0.50%, and 0.75% w/w AgNPs (FIG. 2A). These concentrations were based on estimated conductivities using the percolation theory, which approximates the conductivity of a composite material with conductive fillers (Kim, et al. (2016) Sci. Rep., 6:34632; Gonon, et al. (2006) J. Appl. Phys., 99:024308; Lee, et al. (2018) J. Korean Phys. Soc., 73:152). Using the series of equations (Gonon, et al. (2006) J. Appl. Phys., 99:024308):

V_{f}<Vₚ:σ_{DC}=σᵢ(Vₚ−V_{f})^{-s} (1)

V_{f}>Vₚ:σ_{DC}=σ_{f}(V_{f}−Vₚ)^{t} (2)

where V_{f} is the filler volume fraction, Vₚ is the percolation threshold, σ_{DC} is the conductivity of the composite material in S cm⁻¹, σᵢ is the conductivity of the matrix material in S cm⁻¹, of is the conductivity of the filler in S cm⁻¹, s is the critical exponent, and t is the critical index of conductivity. The percolation threshold, Vₚ, can be calculated as (Gonon, et al. (2006) J. Appl. Phys., 99:024308):

Vₚ=[1+K(Rᵢ/R_{c})]⁻¹ (3)

where K is constant and equal to 0.75, Rᵢ is the radius of the matrix or insulating material, and R_{c} is the radius of the conductive filler. Based on this equation and the use of PCL as insulative fibers with diameter of 20 μm, AgNP with diameter of 70 nm, and critical index, t, of 1.5, fibers should attain a threshold conductivity of 8.97×10⁻⁴ S cm⁻¹ and thus transition from unflockable insulative microfibers to charged flocking fibers. Equations (1)-(3) were used to calculate theoretical conductivity at different AgNP filler concentrations (FIG. 2B). To validate and quantify AgNP encapsulation within PCL microfibers produced by wet spinning, silver was detected using inductively coupled plasma mass spectrometry (ICP-MS) (FIG. 2C). The relationship between loaded and encapsulated AgNP allows for relatively accurate conductive filler loading, such that critical conductive filler volumes can be estimated. Notably, silver leaching occurred in a concentration-dependent manner over a 28-day incubation in PBS at 37° C., with a maximum Ag+ concentration (≈55 ppb) falling far below the concentration at which toxicity is expected in vitro (≈1250 ppb) (FIG. 2C) (Liao, et al. (2019) Int. J. Mol. Sci., 20:2).

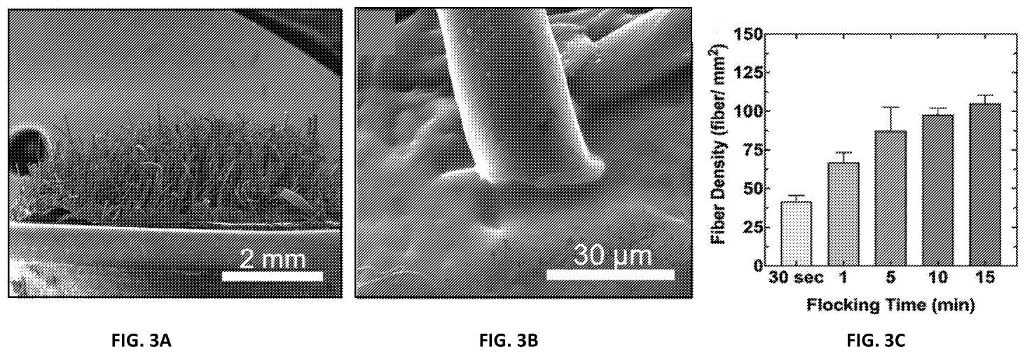

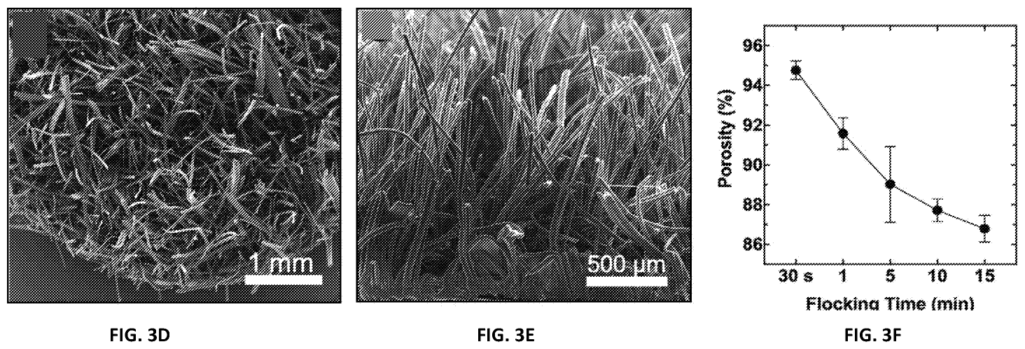

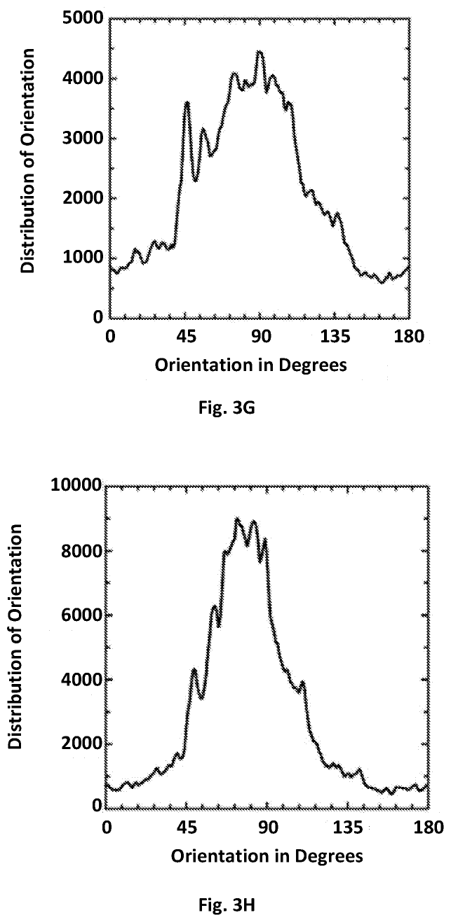

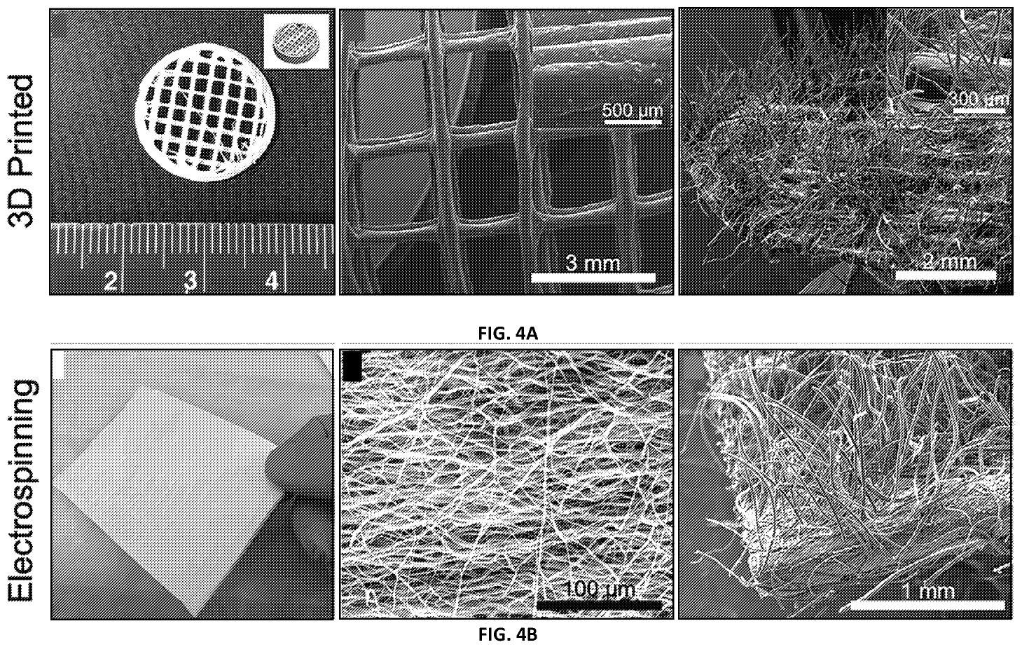

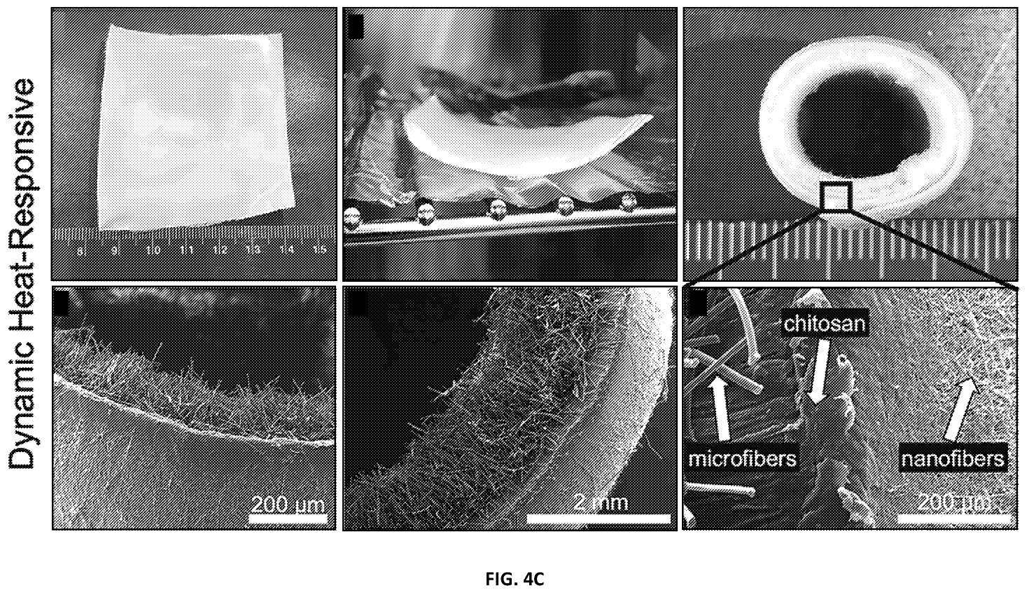

The morphology of scaffolds used for all biological tests (0.5% AgNP/PCL fibers, 1:1 gel:CHS adhesive, flock time=15 minutes) appeared as anisotropic forests of microfibers (FIG. 3A). Fibers embedded and adhered well to the 1:1 gel:CHS adhesive, demonstrating stability and gradual degradation under prolonged incubation in simulated body fluid (SBF) (FIGS. 3B and 4C) (Derakhshandeh, et al. (2020) Adv. Funct. Mater., 30:1905544). Top and side views of scaffolds revealed high fiber density and alignment (FIGS. 3D and 3E). Fiber densities of scaffolds generated at different time points were calculated by quantifying fibers in a given geometric area and scaling for total scaffold area. Not surprisingly, fiber density increased with flocking time and eventually reached maximum packing density at ≈100 fibers mm⁻² (FIG. 3C). Similarly, porosity (measured as volume fraction of air) was quantified using a reported equation, with porosity linearly decreasing with flock time from roughly 95% to 85% (FIG. 3F) (Gossla, et al. (2016) Acta Biomater., 44:267; Tonndorf, et al. (2018) Text. Res. J., 88:1965). Finally, anisotropy was analyzed and quantified using tensor analysis and compared to a control (Rayon with a proprietary flocking finish) used in industrial flocking applications (FIGS. 3G and 3H). Quality of alignment between Rayon and 0.50% AgNP/PCI, fibers was extremely similar, with both fibers achieving relatively high alignment (K-S D=0.3860, peak frequency=ns) (FIGS. 3G and 3H).

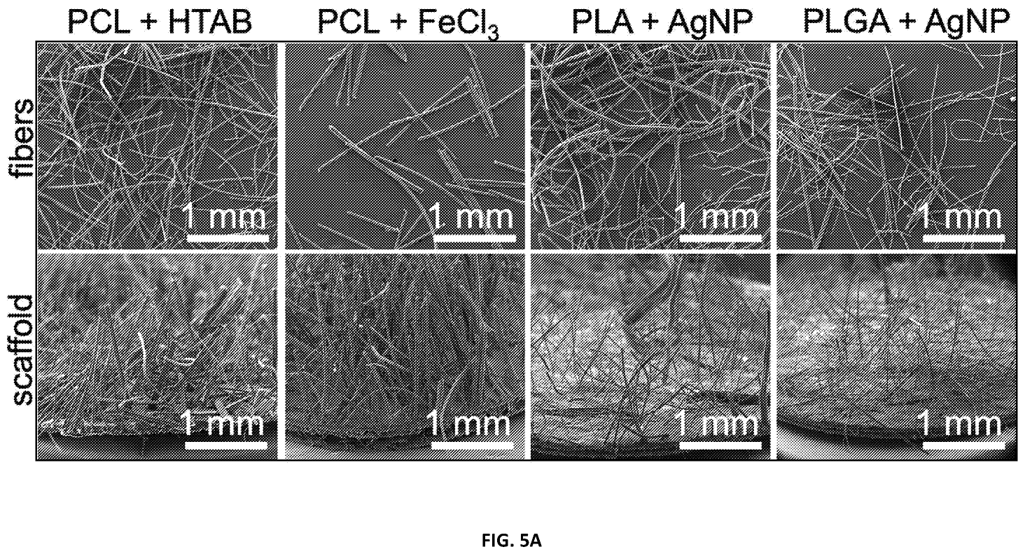

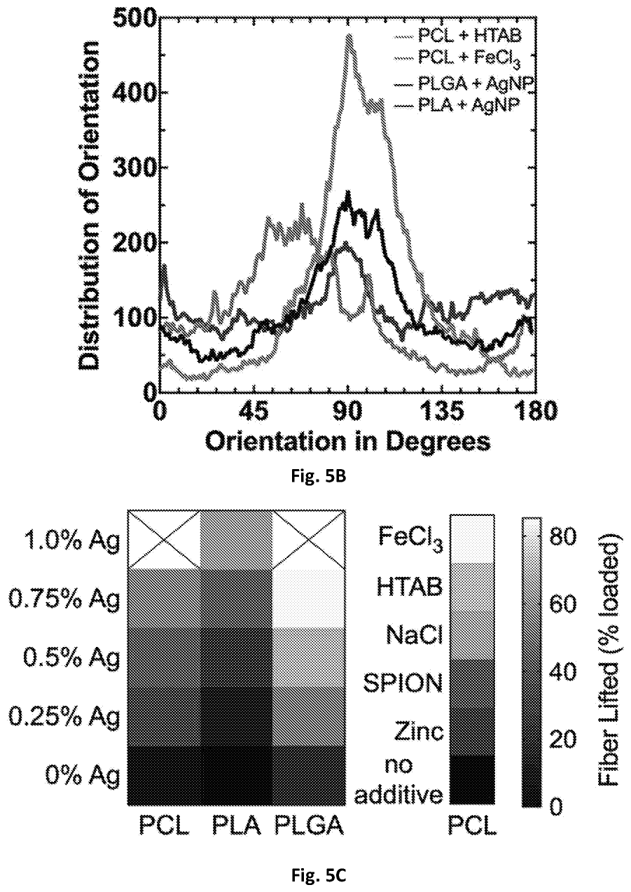

To demonstrate flexibility of flocking for biomedical applications, flocked scaffolds utilizing 3D-printed and electrospun nanofiber membranes were generated. Hydroxyapatite (HA)/PCL circular, multilayer meshes were produced by extrusion-based jet printing (FIG. 4A). Similar 3D-printed scaffolds have been used in cranial and mandibular bone regeneration (Aldrich, et al. (2019) ACS Appl. Mater. Interfaces, 11:12298; Zhang, et al. (2020) Biofabrication 12:035020). Aligned electrospun PCL nanofiber membranes were prepared following reported methods, and have broad biomedical applications (FIG. 4B) (Chen, et al. (2018) J. Mater. Chem. B, 6:393; Carter, et al. (2016) J. Biomater. Sci., Polym. Ed., 27:692; Su, et al. (2020) ACS Nano 14:11775; Sarasam, et al. (2005) Biomaterials 26:5500). Both 3D-printed HA/PCL and electrospun PCL scaffolds were used as flocking substrates and subsequently dipped in 1:1 gel: CHS adhesive, affixed to the ground electrode, and coated with flocked 0.50% AgNP/PCL flocking fibers. After flocking, 3D-printed scaffolds retained their original millimeter sized pores while exhibiting a single-faced, anisotropic flock fiber finishing (FIG. 4A). In application, flock fibers can act as anchor points for cell proliferation, guiding cells toward the 3D-printed meshes and accelerating proliferation and integration into the bulk 3D-printed substrate. Similarly, the nanofibrous topological morphology of flocked electrospun nanofiber membranes was retained after flocking with 0.50% AgNP/PCL microfibers (FIG. 4). Flocked nanofiber scaffolds exhibited a biphasic structure, with microfibers positioned perpendicular to the aligned nanofibers. Objects with biphasic structures are particularly useful in regenerative medicine as they allow for precise directional control over cells at implant sites (Fan, et al. (2018) ACS Nano, 12:5780; Lee, et al. (2016) RSC Adv., 6:79900; Shah, et al. (2011) Ann. Biomed. Eng., 39:2501). To demonstrate the literal flexibility of flocking, a self-folding, triphasic, and hierarchical conduit was fabricated using highly aligned electrospun PCL nanofiber membranes, 1:1 gel:CHS adhesives, and poly(lactide) (PLA) flocking fibers (FIG. 4C). Since PCI, has a lower melting temperature than PLA, gently heating (65° C., 30 minutes) the flocked construct initiated folding along the nanofiber direction without melting or deforming PLA flock fibers (FIG. 4C). After 24 hours, the conduit had formed a cylinder with flock fibers projecting inward (FIG. 4C). Scanning electron microscopy (SEM) images revealed three distinct phases: microfibers (core), gel:CHS adhesive (fiber interface), and nanofibers (conduit walls) (FIG. 4C). Such a device may serve as an skeletal muscle graft/anchoring device or intestine mimicking material, as the outer nanofiber sheath may prevent other cell lineages from infiltrating the conduit, but the interior flocking fibers can serve as anchor points and proliferative guides for designated cell types (Xing, et al. (2017) Adv. Healthcare Mater., 6:1601333; Ryan, et al. (2017) Adv. Healthcare Mater., 6:1700954; Abdal-hay, et al. (2018) Mater. Sci. Eng., 82:10). Finally, to provide a first-of-kind reference for others in material science, a brief, biomedically relevant library of different polymers and conductive treatments for creating flockable fibers was established (FIG. 5). Using the aforementioned methods, fibers made from PCL, PLA, and poly(lactic-co-glycolic acid) (PLGA) and treated with hexadecyltrimethylammonium bromide (HTAB), ferric chloride (FeCl₃), AgNPs, zinc powder, sodium chloride (NaCl), and superparamagnetic iron oxide nanoparticles (SPIONS) were used to create flocked scaffolds (FIG. 5A). Fiber alignment and mass lifted during flocking was quantified for reference for filler/polymer-specific applications (FIGS. 5B and 5C).

After outlining fabrication, characterization, and flexibility of flocking with percolation theory-induced conductivity, biological activity and applications of flocked scaffolds were studied. Herein, flocked scaffolds composed of 0.50% AgNP/PCL flock fibers, 1:1 gel:CHS adhesives/substrates were evaluated for mechanical stability, antimicrobial efficacy, cytotoxicity, in vitro cellular response, and in vivo response following subcutaneous implantation.

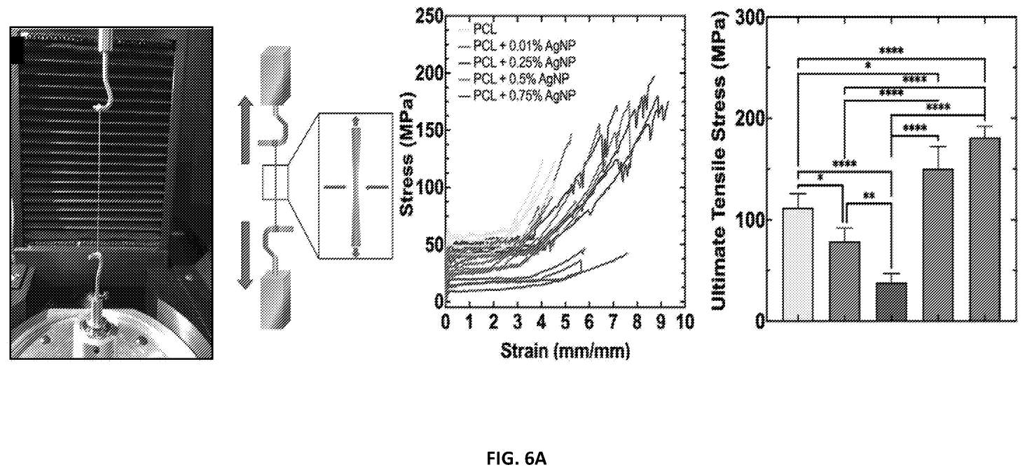

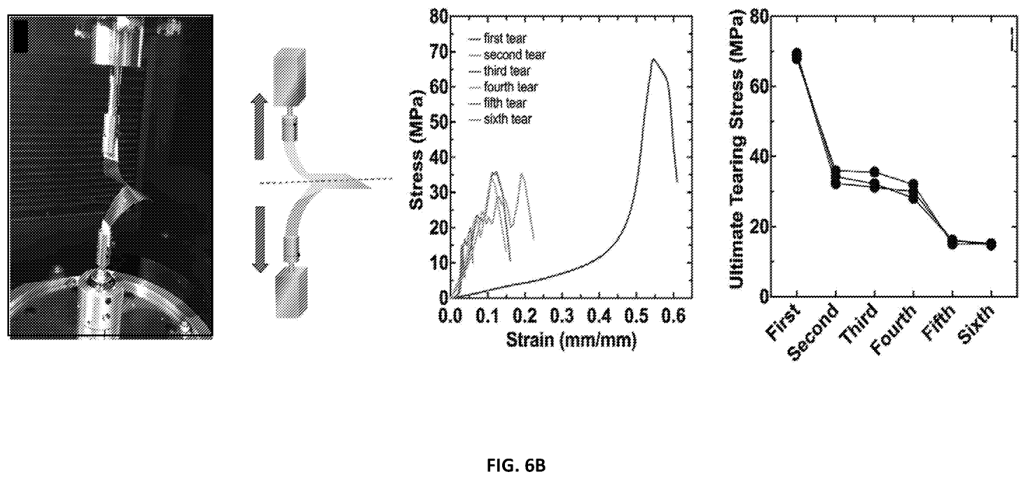

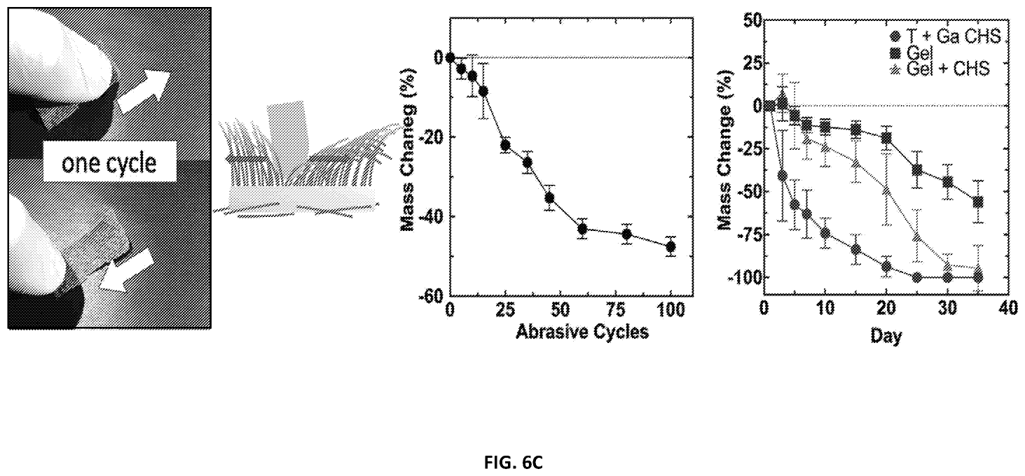

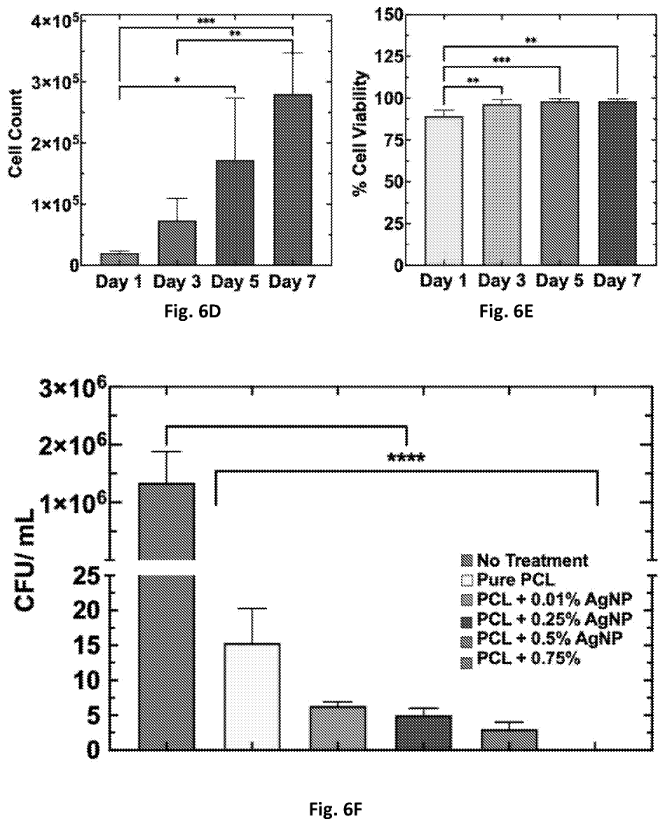

Mechanical stability of flocked scaffolds is particularly relevant in load-bearing, shearing, or abrasive applications (e.g., cartilage engineering, skin tissue engineering, long bone engineering). Flock fibers should retain elasticity to withstand tissue deformations that could break and fragment brittle fibers. Uniaxial tensile testing on fibers with different AgNP concentrations revealed that small filling loads of AgNP decreased ultimate tensile stress (UTS) up to a certain concentration, and gradually increased UTS at higher filling loads (FIG. 6A). This disruptive phenomenon is likely explained by high filler fractions that establish necking resistance (Kord, J. (21012) Thermoplast. Compos Mater., 25:793). Similarly, a trouser tear test with multiple iterations on GA-crosslinked 1:1 gel:CHS substrates revealed that the tear resistance of flocked scaffold adhesive/substrate complexes was approximately double the tear resistance of natural human upper back skin (FIG. 6B) (Annaidh, et al. (2012) J. Mech. Behav. Biomed. Mater., 5:139). Stability of the scaffold during linear abrasion was investigated using a “thumb test,” a common abrasion resistance test in the flocking industry Scaffolds showed modest abrasion resistance after 25 cycles and the majority of the mass loss by way of fiber loosening. Degradation of flocked scaffolds using three different adhesives (GA-crosslinked gel, GA-crosslinked gel+CHS, and thermally cured and GA-crosslinked CHS only) was analyzed in body simulating fluid (BSF), with GA-crosslinked gel+CHS having the most desirable degradation rates (˜50% by day 20).

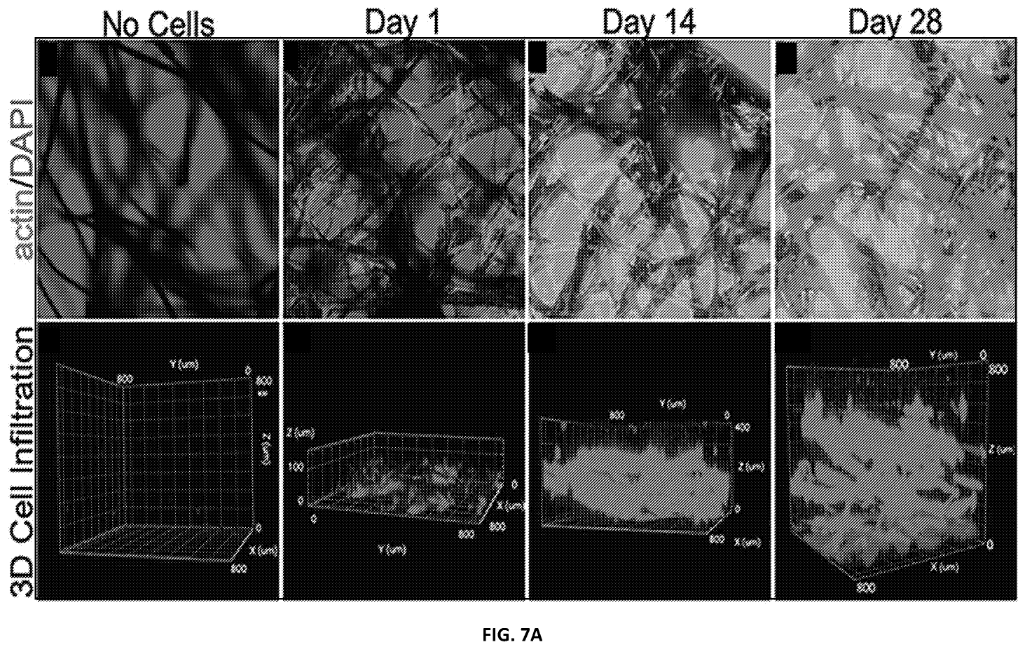

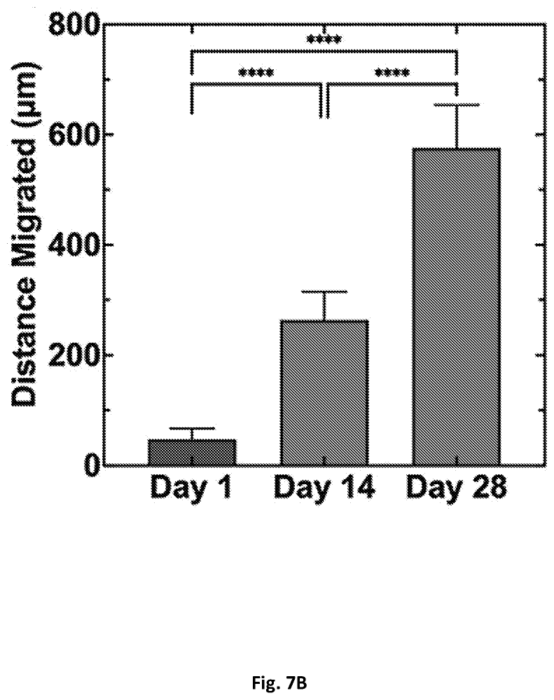

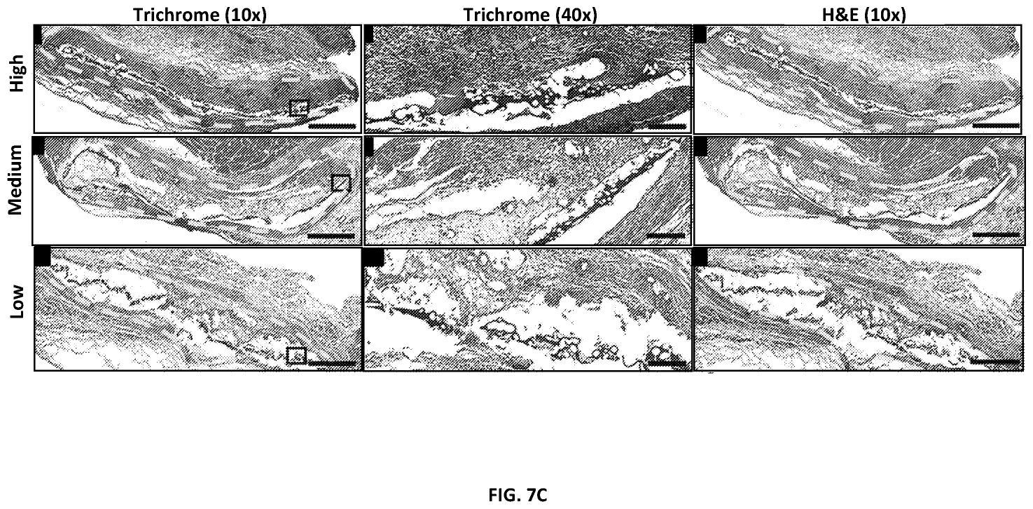

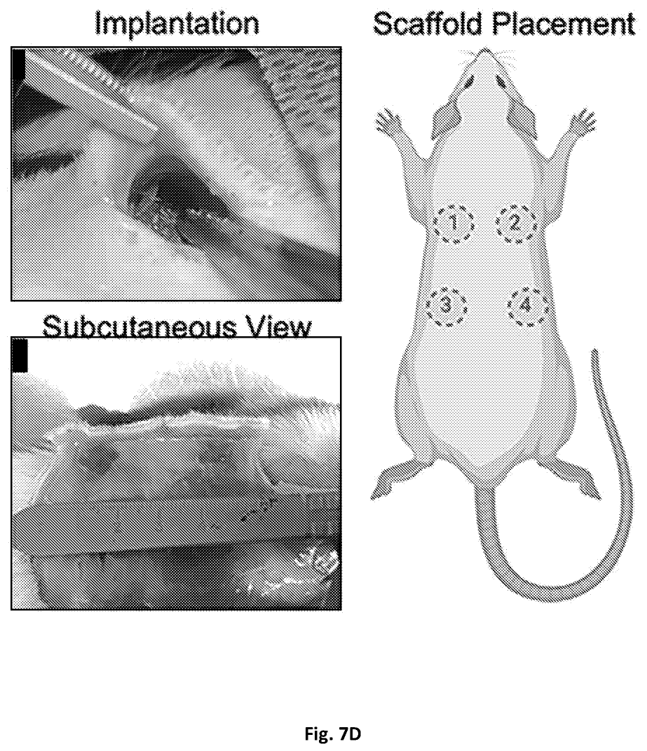

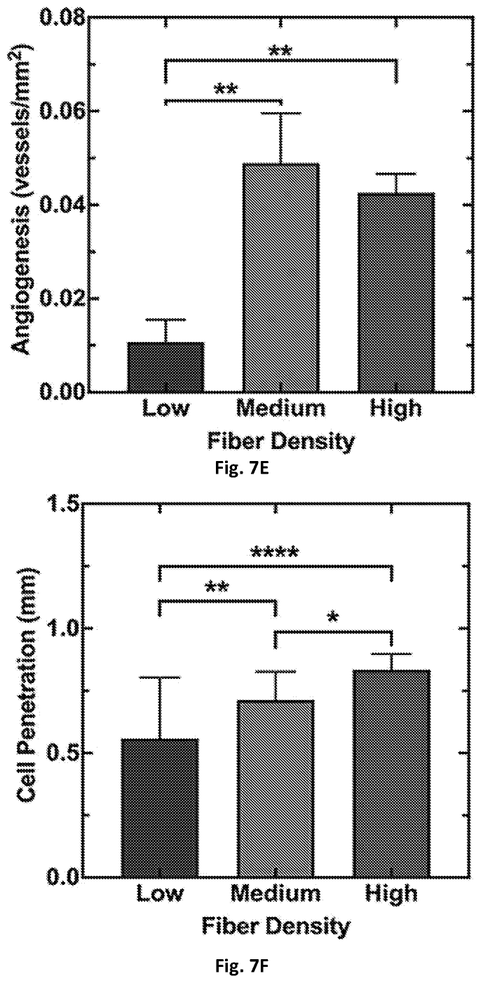

Considering the anisotropic nature of flocked scaffolds, the flock fibers can act as topographic cues for cells, guiding their migration and sustaining proliferation. Ultimately, flocked scaffolds can sustain full tissue formation and increase the rate of cell infiltration in defect sites. To evaluate the biological function of the flocked scaffolds, rat bone marrow-derived mesenchymal stem cells (rBMSCs) were seeded onto ethylene oxide-sterilized flocked scaffolds and allowed to proliferate for 7 days before imaging. Cells seeded onto the scaffold showed significant proliferation and retained high viability, with migratory patterns similar to those reported by Gossla et al. (migrating from the substrate base to fiber tips) (FIGS. 6D and 6E) (Gossla, et al. (2016) Acta Biomater., 44:267). Further, flocked scaffolds had AgNP dose-dependent antimicrobial efficacy against Methicillin-resistant Staphylococcus aureus (MRSA), reducing colonies from ≈1.5×10⁶ CFU mL⁻¹ to less than 8 CFU mL⁻¹ for all scaffolds incorporating Ag VPs. Antimicrobial activity appeared imparted by both AgNPs and the natural antimicrobial properties of CHS, though CHS served as the primary source of antibacterial activity (FIG. 6F) (Su, et al. (2020) ACS Nano 14:11775; You, et al. (2017) Sci. Rep., 7:10489; Kim, et al. (2017) Nano Convergence 4:33). Tissue formation on flocked scaffolds was mapped over a 28-day period using actin/DAPI-stained rBMSCs and 3D confocal imaging and average distance migrated was measured. After 28 days of culture, cells migrated away from the base of the substrate, forming successive tissue layers that extended nearly 800 μm from the substrate (FIGS. 7A and 7B). The results from the tissue formation study confirmed that the flock fibers act as migratory guides for proliferative cells.

Noting the salutary in vitro effect on proliferation, viability, antimicrobial efficacy, and tissue-forming capability, in vivo responses were investigated by subcutaneously implanting flocked scaffolds in rats. Scaffolds with varying fiber densities were subcutaneously implanted on rat dorsum, with fibers superficial to the substrate (FIG. 7D). After 7 days, tissue surrounding the flocked scaffolds (area outlined) was removed and analyzed for cell infiltration and angiogenesis (arrows) (FIG. 7C). Cell penetration and angiogenesis occurred in a fiber density-dependent manner. New vessel formation and improved cell infiltration was greater in tissues with medium and high fiber density scaffolds compared to low fiber density scaffolds. The flock fibers and flocked scaffold served as anchoring points that could promote cellular migration from the surrounding native tissue into the defect (FIGS. 7E and 7F). The results from the in vivo study demonstrated that 1) implanted flocked scaffolds could withstand local shearing forces under skin, 2) angiogenesis occurred in a fiber density-dependent manner, and 3) cell infiltration was accelerated in a fiber density-dependent manner.

Flocked scaffolds can improve cell survivorship under mechanical compression and sustain cell viability and proliferation under culture conditions. Interlocking biomaterials can be generated by electrostatic flocking and further reinforced by extracellular matrix secreted by seeded cells. The introduction of an extracellular matrix-reinforced interlocking biomaterial enables modular tissue engineering, act as reversibly-interlocking substrates, or enable friction-based couplers for biomedical applications.

As demonstrated in this work, electrostatic flocking utilizing charge accumulation based on the percolation theory is a widely applicable method for creating anisotropic surfaces or standalone objects. In general, electrostatic flocking is quick process that is easy to scale up and is limited only by an objects' ability to be coated by an adhesive. Compared to other systems, such as freeze-casting or electrospinning, electrostatic flocking is a much faster technique that has increased functionality and broader applicability.

In summary, the data presented herein supports the use of electrostatic flocking as a broadly applicable technology by detailing multiple methods for flocking insulative polymer microfibers. For the first time, the fabrication of flocked scaffolds using insulative biopolymers has been demonstrated by incorporating conductive filler treatments based on principles from the percolation theory. A library of different fillers and polymers was generated, as well as estimated necessary AgNP loading content to achieve flockability in PCL microfibers. The broad applicability of this technique was highlighted by surface flocking 3D-printed HA/PCL meshes and PCL nanofiber membranes and creating a novel triphasic, self-folding conduit. In addition to generating data supporting the biocompatibility, mechanical stability, and antimicrobial efficacy of flocked scaffolds, tissue formation in vitro and in vivo was generated, which can be modulated by altering flock fiber densities. Electrostatic flocking may serve as a renewed and highly applicable shift in biomaterial and surface coatings, as its use is limited only by the size of the substrate/object to be flocked. Flocked fibers can act as forests of cell/tissue anchors in a variety of implantable applications, such as hernia meshes, soft tissue prosthetics, cranial defect scaffolds, and cartilage scaffolds. Outside of biomedical applications, flocked surfaces utilizing the methodology has applications in adhesives, composite materials, renewable energy, and surface chemistry as a broad field (Tu, et al. (2019) Small 15:1902070; Uetani, et al. (2014) Adv. Mater., 26:5857; Yu, et al. (2012) Langmuir 28:8746; Zheng, et al. (2018) RSC Adv., 8:10719).

### Example 2

Creating highly porous and anisotropic objects for regenerative medicine and tissue engineering has remained a hallmark goal for several decades. Ideally, objects would retain high porosity and exhibit superb mechanical strength; however, this is rarely the case as highly porous materials (i.e., sponges or foams) have poor mechanical strengths and may be ill-suited in load bearing applications (Fang, et al. (2020) Chem. Eng. J., 388:124169). Increased efforts to generate anisotropic and highly porous biomaterials have resulted in new materials and techniques. For example, aligned 3D nanofiber objects, anisotropic aerogels, anisotropic and freeze-cast sponges, and electrostatically flocked objects have all demonstrated high porosities and salutary mechanical properties (Chen, et al. (2019) Nano Lett., 19:2059-2065; John, et al. (2021) Adv. Healthc. Mater., 12:2100238; Wang, et al. (2018) ACS Nano 12:5816-5825; Balasubramanian, et al. (2015) Mater. Lett., 158:313-316; Gossla, et al. (2016) Acta Biomater., 44:267-276; McCarthy, et al. (2021) Nano Select 2:1566-1579). Notably, electrostatic flocking is a textile technology that uses an electrical field and Coulombic driving force to charge and launch fibers towards a substrate covered with an adhesive (Kim, Flocked fabrics and structures, in: Specialist Yarn and Fabric Structures, Elsevier, 2011, pp. 287-317). Electrostatic flocking typically consists of three components: short (micrometers to millimeters in length) fibers (flock fibers), adhesives, and substrates (Gossla, et al. (2016) Acta Biomater., 44:267-276). Prior to flocking, fibers are sieved onto a charging electrode, where voltage is gradually applied until a sufficient charge is accumulated on the fiber surfaces. Once sufficient charging is achieved, the fibers are launched towards the ground electrode and embedded into an adhesive-covered substrate, leaving a forest of aligned fibers (Uetani, et al. (2014) Adv. Mater., 26:5857-5862). Fibers can be directly embedded into adhesives or cover the surface of an adhesive-covered substrate. Parameters such as flocking time, fibers sieved, fiber morphology, and applied voltage can be used to control fiber densities (Gossla, et al. (2016) Acta Biomater., 44:267-276). After fibers are sufficiently embedded, the adhesive/substrate can be easily removed from the ground electrode. Electrostatic flocking parameters including flock time, fiber length, and applied voltage have been optimized and fiber density and porosity can be easily controlled by flock time (the time a voltage is applied to the charging electrode) and fiber length (Tonndorf, et al. (2018) Textil. Res. J., 88: 1965-1978; Walther, et al. (2012) Materials 5:540-557). These studies found that fiber density increased with flocking time and decreased with fiber length. One mm long fibers flocked for 5 and 15 seconds achieved fiber densities of 72±11.2 and 104.5±10.7 fibers/mm², respectively, while 3 mm fibers flocked for 5 and 15 seconds achieved only 11.4±1.8 and 21.33±3.3 fibers/mm², respectively (Walther, et al. (2012) Materials 5:540-557).

Electrostatic flocking has been used in tissue engineering as a fabrication technique to create porous and anisotropic structures for osteochondral engineering (Gossla, et al. (2016) Acta Biomater., 44:267-276; Tonndorf, et al. (2018) Textil. Res. J., 88:1965-1978; Walther, et al. (2012) Materials 5:540-557). Noting the favorable in vitro and in vivo responses and tunable porosity and fiber density, flocked materials offer an advantage over other tissue engineering and regenerative medicine approaches in some applications. The establishment of fully degradable flocked scaffolds using both chitosan and poly(-ε-caprolactone) (PCL) demonstrated that inherently or charge-modified fibers could effectively accumulate charge necessary for flocking (Gossla, et al. (2016) Acta Biomater., 44:267-276). Charge accumulation typically relies on the hydrophilicity of the flock fiber surfaces (Gouveia, et al. (2009) J. Am. Chem. Soc., 131:11381-11386). To this end, most fibers have a proprietary finish applied to them that allows for rapid charge accumulation. Hydrophobic and electrically insulative polymers can accumulate sufficient charge by utilizing conductive fractional fillers following principles of the Percolation Theory (Example 1; Kim, et al. (2016) Sci. Rep., 6:34632). Another method to induce conductivity on a fiber surface may be the implementation of a salt-based system that ionizes under sufficient humidities (Ingamells, et al (2008) J. Soc. Dye. Colour., 108:270-278). Salt ions hold potential for increasing surface conductivity of objects and in solutions (Golnabi, et al. (2009) Iran, Phys. J., 3:6; Kiran, et al. (2012) J. Non-Cryst. Solids 358:3205-3211). Herein, methods for fabricating monofilament microfibers (MFs) from wet spinning and nanofiber yarns (NFYs) from electrospinning to create electrostatically flocked scaffolds utilizing a NaCl treatment system.

One use case for electrostatically flocked scaffolds is towards wound healing. Structural anisotropic wound healing scaffolds have shown promise towards accelerating wound healing, promoting angiogenesis, and inducing granulation tissue formation (Lu, et al. (2018) ACS Appl. Mater. Interfaces 10:44314-44323; Chen, et al. (2020) Acta Biomater., 108:153-167). Given the continued fiscal and patient cost of wound treatment, efforts towards advancing wound healing scaffolds towards clinical relevancy persists (Landsman, et al. (2019) Value Health 22:S213; Han, et al. (2017) Adv. Ther., 34:599-610). The high porosity and surface area-to-volume ratio of flocked scaffolds make them appealing wound healing scaffolds as they can allow for nutrient and oxygen circulation within the wound while serving as scaffolds for new tissue formation. To this end, the use of flocked scaffolds as wound healing treatments in type 2 diabetic mice was evaluated. In addition to using standalone flocked wound healing scaffolds, the concept of flock-reinforced elastomers for biomedical applications was also used.

One application for flock-reinforced objects is in the production of short fiber-reinforced elastomeric composites (Uetani, et al. (2014) Adv. Mater., 26:5857-5862; Yu, et al. (2019) J. Mater. Sci. Mater. Electron., 30:10233-10243). Anisotropic composite elastomers offer an advantage in the treatment of lumbar disc degenerative disease (DDD) (Volpe, et al. (2020) Adv. Healthc. Mater., 9:e1901136; Khandaker, et al. (2017) J. Healthc. Eng., 5283846,), a pathology in which the breakdown of an intervertebral disc leads to structural instability and chronic pain (Dowdell, et al. (2017) Neurosurgery 80:S46-S54). The intervertebral disc is located between all vertebral bodies within the spine, excluding C1 and C2 (atlas and axis, respectively), and is identified by the two vertebral bodies in which it separates (Humzah, et al. (1988) Anat 220 (1988) 337-356). The disc is composed of three major structures: the annulus fibrosus, the nucleus pulposus, and cartilaginous endplates (Kim, et al. (2020) Int. J. Mol. Sci., 21:1483). The annulus fibrosus is organized in an alternating, crisscross pattern of type 1 and type 2 collagen strands, varying in angle of orientation, forming an angle-ply structure (Kim, et al. (2020) Int. J. Mol. Sci., 21:1483; Martin, et al. (2014) Acta Biomater., 10:2473-2481). The collagen matrix produces an anisotropic system designed to withstand vertical compression and fluctuating radial loads, as well as allow for multidirectional torsion (Kim, et al. (2020) Int. J. Mol. Sci., 21:1483; Smith, et al. (2009) Eur. Spine J., 18:439-448). Obstruction of nutrient supply to these cells has been shown to lower oxygen concentration, decrease pH, and impair the ability of the disc to respond and heal from structural strain, highlighting a need to stress-shield the cells within the intervertebral disc (Dowdell, et al. (2017) Neurosurgery 80:S46-S54). A variety of treatments exist for each stage of the disease progression, yet total disc replacement (TDR) is standard for patients who have failed prior conservative treatment (Formica, et al. (2020) Eur. Spine J., 29:1518-1526). TDR is preferred in comparison to spinal fusion at the level of the degenerated disc, allowing for restoration of normal disc height and function to alleviate pain without compromising range of motion (Salzmann, et al. (2017) Curr. Rev. Musculoskelet. Med., 10:153-159). Current artificial discs are manufactured with a core of ultra-high molecular weight polyethylene (UHMWPE), a stiff material designed to withstand significant uniaxial compressive forces and resist delamination (Othman et al. (2019) Ann. Transl. Med., 7(Suppl 5):S170). Although the stiffness of UHMWPE is desirable from a uniaxial compression standpoint, the material fails to support multiaxial loads or provide shock absorbance for the vertebrae, leading to disc failure at various stress points (Martin, et al. (2014) Acta Biomater., 10:2473-2481; Othman et al. (2019) Ann. Transl. Med., 7(Suppl 5):S170). In an effort to mirror the anatomical organization of an intervertebral disc, a structure similar to the angle-ply, layered construction of the annulus fibrosus, generated using polydimethylsiloxane (PDMS) polymer composites and electrostatically flocked PCL microfibers can be used.

Herein, a high-fidelity, easy, and cost-effective method to induce surface charge accumulation on electrically insulative short polymer fibers to achieve high-yield electrostatic flocking is provided. To validate the reproducibility, multi-polymer functionality, and demonstrate the broad biomedical and biomechanical role of electrostatic flocking, three polymer types (PCL, poly(lactic-co-glycolic acid) PGLA, and polylactic acid (PLA)) were used to create standalone NFY and MF scaffolds, low and high density wound healing constructs, and flock fiber-reinforced artificial vertebral discs (AVDs), respectively (Table 1). The mechanical, biological, and biomechanical effectiveness of flocked scaffolds and AVDs are evaluated herein.

TABLE 1
Different scaffold compositions and use cases reported herein.

| Name | Polymer | Fiber Form | Substrate | Application |
|---|---|---|---|---|
| MF | PCL | Microfiber | Chitosan/ | In vitro cell scaffold |
| | | | gelatin | |
| NFY | PCL | Nanofiber | Chitosan/ | Proof of concept |
| | | yarn | gelatin | |
| whMF | 90:10 | Microfiber | Chitosan/ | In vivo wound healing |
| | PGLA | | gelatin | |
| avdMF | PLA | microfiber | PDMS | Artificial vertebral |
| | | | | disc reinforcement |

MF: microfiber; NFY: nanofiber yarn; whMF: microfibers for wound healing; avdMF: microfibers for artificial vertebral disc.

### Materials and Methods

### Production of Nanofiber Yarns

NFYs were produced by first electrospinning 10% w/v PCL (Mw=80 kDa, Sigma-Aldrich, St. Louis, MO) dissolved in a 4:1 (v/v) mix of dichloromethane to N, N-dimethylformamide (DCM:DMF) (BDH Chemicals, Dawsonville, GA) as described (Chen, et al. (2017) Nanomedicine 13:1435-1445). After the polymer solution was homogenous, it was loaded into a 10-cc syringe capped with a blunted 21-gauge needle. The polymer solution was electrospun under ambient conditions (21.6° C.; relative humidity (RH)=45%) with an applied voltage of 18.0 kV and a flow rate of 0.8 ml/h. During electrospinning, nanofibers were collected on a high-speed rotating drum (7 mm in diameter). After 1 ml of the polymer solution was collected on the drum, the fiber mat was cut into 3-15 mm strips and rolled along the drum to form the NFYs. Using a razor blade, the yarn was cut and removed from the drum.

### Wet Spinning of Continuous Fiber Tow

Continuous PCL fibers were prepared with phase-separation wet spinning. First, 20% w/v PCL (Mw=80 kDa, Sigma-Aldrich, St. Louis, MO) and 0.5% w/v F-127 (Sigma-Aldrich, St. Louis, MO) were dissolved in a 4:1 (v/v) mix dichloromethane to N, N-dimethylformamide (DCM: DMF) (BDH Chemicals, Dawsonville, GA) and stirred until a homogeneous solution with uniform viscosity was achieved. Next, a 10-cc syringe capped with a 21-gauge needle was loaded with the polymer solution and extruded under ambient conditions (21.6° C.; RH=45%) with a coagulation bath of 70% EtOH at a rate of 0.3 ml/hour. Fibers were collected on a rotating drum with take-up rates ranging from 5 to 25 RPM. After fibers were collected, they were removed with a razor blade and allowed to air dry. Similarly, PGLA (90:10) (Mw=60 kDa, DURECT Corporation, Pelham AL) fibers were produced by creating 25% (w/v) PGLA solution dissolved in a 4:1 (v/v) mix of DCM:DMF and extruded under the same conditions as the PCL fibers. In the case of direct extrusion into an NaCl bath, NaCl (0.65 g NaCl/kg ethanol) was added to the bath and the above process was carried out as described.

### Producing NaCl-Treated Flock Fibers

After the MF and NFYs were dried, they were mechanically cut using surgical scissors at approximately 1-2 mm in length. Untreated PLA flock fibers were supplied as pre-cut by Spectro Coating Corp. To separate any fused MF ends, the fibers were plasma treated for 3 minutes and suspended in an ice water bath. Next, fibers were homogenized in a NaCl-saturated (1.2 g/ml) ice water bath using a probe tip ultrasonicator for 40 minutes with a 30 seconds pulse/10 seconds rest cycle. It is important to note that separation using sonication methods requires careful maintenance of temperature, depending on the polymer used, as the fibers may fuse together under high heat conditions. Following separation, fibers were collected with vacuum filtration. NFYs did not undergo separating steps but underwent the same plasma and salt wash prior to flocking. The resulting NFYs and MFs were allowed to air dry and were stored at 23° C. and 55% RH until use. All fibers (PCL, PLA, and PGLA) were separated and treated with NaCl as described in this section.

### Electrostatic Flocking

In both MF and NFY scaffolds, the adhesive layers served as the substrates. For in vitro and in vivo studies, a 20% w/v gelatin/H₂O solution was prepared by dissolving 2 g gelatin in 10 ml of deionized H₂O. Similarly, a chitosan (CHS) solution was prepared by dissolving 0.5 g of CHS in 10 ml of 5% w/w acetic acid/H₂O. Both solutions were heated at 60° C. until both solutions were homogenous. Then, a 50/50 blend of the CHS and gelatin solutions was created and stored at ambient temperatures until further use. For AVD tests, PDMS was prepared at a 10:1 base: curing agent ratio. In both cases, the adhesive layer was razor coated on the ground electrode such that a thin layer of uniform adhesive remained. MFs and NFYs were removed from humidity-controlled storage (RH=40%) and MFs (NFYs did not require sieving) were sieved onto the charging electrode with a mechanical sieve. With a distance of 10 cm between each electrode on a MaagFlock SPG 1000 electrostatic flocking machine (Maag Flock GmbH, M€ossingen, Germany), an increasing voltage of 30-60 kV was applied until no fibers remained on the charging electrode. Scaffolds prepared with CHS/gelatin adhesives were thermally cured for 5 minutes at 60° C. and dried at ambient conditions overnight before crosslinking in a glutaraldehyde chamber for 24 hours. Scaffolds prepared with PDMS were heat cured at 50° C. for 24 hours. Before use, loose fibers were removed with an air hose and scaffolds were incubated in 70% ethanol for at least 24 hours to sterilize and remove any remaining loose fibers. Scaffolds used for in vitro and in vivo testing were sterilized using ethylene oxide (EtO) gas sterilization overnight. The use of EtO sterilization and ethanol incubation ensured high sterility while retaining fibrous and anisotropic morphologies.

### Flock Yield

Prior to measuring flock yield, fibers were incubated for 24 hours at each RH that was tested. RH was controlled in the flocking chamber by using a Hunter 31004 Cool Mist Ultrasonic Humidifier (Hunter Fan Company, Memphis, TN) with deionized H₂O and RH readings were taken with a RH meter (Fisherbrand™ Traceable™, Fisher Scientific, Waltham, MA). Flock yield was calculated as the fraction of fibers lifted relative to the number of fibers sieved onto the charging electrode.

Flock Yield (% fibers lifted)=(ms−mf)/ms×100%

Where mf is the mass of fiber flocked and ms is the mass of fibers sieved. First, fibers were sieved onto the charging electrode and the mass was taken (ms). After flocking, the charging electrode was removed again and reweighed (mf). In most cases, some fibers remained on the surface of the electrode, so it was necessary to take caution during transfer as to not lose fibers to air flow. The difference in masses after flocking was used to calculate the percentage of fibers lifted. While this equation is useful to generally understand flocking yield, the equation does not account for fibers that do not adhere into the adhesive. These fibers may launch into the surrounding flock chamber and would contribute to an overestimation of flock yield.

### Fiber and Scaffold Characterization

Fibers and scaffolds were imaged with a scanning electron microscope (FEI Quanta™ 200, FEI Company, Hillsboro, OR, USA), camera (iPhone 8 Plus, Apple, Palo Alto, CA), or USB microscope (AmScope, Irvine, CA). Length measurements were taken using NIH ImageJ's measure analysis tool, with pixels calibrated to each measurement scale bar. Similarly, fiber diameters were measured using open-source ImageJ plugins SkeletonJ and DiameterJ. First, SEM images of each fiber were taken. SkeletonJ was used to create skeleton images with which DiameterJ used to estimate mean diameters with (McCarthy, et al. (2021) Nano Select 2:1566-1579). Scaffold fiber orientation was calculated by analyzing SEM images with the ImageJ plugin OrientationJ, which uses tensor analysis to quantify mean orientations of the image field (Rezakhaniha, et al. (2012) Biomech. Model. Mechanobiol. 11:461-473). Compression resistance of PCL NFY and MF scaffolds were conducted on a CellScale Uni Vert (CellScale, Ontario, CA) utilizing a 200 N loadframe. Four cyclic compressions to 50% were conducted under ambient conditions with 10 seconds between each compressive load. Abrasion resistance was measured using a modified and standardized thumb (rub) test, which is a flocking industry standard (Kim, Flocked fabrics and structures, in: Specialist Yarn and Fabric Structures, Elsevier, 2011, pp. 287-317). A plastic, round-edged stir bar was placed horizontally over the flocked surface and rubbed back-and-forth with a consistently applied pressure. After each set number of abrasive cycles, the mass of the flocked sample was taken and a change in mass was calculated.

### Anatomically Accurate L4-L5 Model Preparation

Anatomically accurate L4 and L5 vertebrae were created using additive manufacturing. First, de-identified computed tomography (CT) scans of human L4 and L5 vertebrae were accessed via an open-access website, Thingiverse™, as STL file types. The STL files were originally supplied by the Body Parts3D database under a creative commons attribution. Next, the STL files were transferred to a LulzBot Mini v2.0 (Fargo Additive Manufacturing Equipment 3D, LLC, Fargo, ND) for 3D printing. Models were printed using 3 mm diameter poly lactide (PLA) filament (MatterHackers Inc, Lake Forest, CA) at 50% infill density at a 40 mm/second rate, with a wall thickness of 1 mm. Printing was conducted with a nozzle temperature of 220° C. and a build plate temperature of 70° C. A support was generated for stability during the printing process. After printing, support material was removed, and the vertebrae were used for disc preparation.

### Creating Artificial Discs

To create AVDs, round molds (diameter=55 mm, height=8 mm) were 3D printed using the above-mentioned parameters and materials. For control discs (no fiber reinforcement), 11 ml of 10:1 elastomer base:curing agent polydimethylsiloxane (PDMS) (Sylgard™ 184 Silicone, Dow Corning, Midland, MI) was poured into the mold and cured in at 65° C. over for 24 hours to reach a final disc height of 7 mm. PDMS was used as a model elastomer due to its low cost, mechanical robustness, ease of use, and lengthy history in biomedical applications, particularly as a component in lumbar disc replacements (Fischer, et al. (2016) J. Mech. Behav. Biomed. Mater., 61:87-95; Alvarez, et al. (2019) J. Mech. Behav. Biomed. Mater. 91:383-3). After removal from the mold, the PDMS discs were die-cut using a custom cut mold based to fit between the L4 and L5 vertebrae. To create the fiber-reinforced discs, a small amount of PDMS was knife-coated on the ground electrostatic flocking electrode. A monolayer of NaCl/PLA fibers (diameter=20 μm, length=0.5 mm) was sieved onto the charging electrode and flocked upwards towards the PDMS layer for 1 minute with the applied voltage gradually increasing from 30 kV to 60 kV. After flocking, the ground electrode was removed and allowed to cure. The flocked PDMS was cut to fit into the PDMS mold and enough PDMS was added to barely cover the top of the fibers. This was repeated until the final height of the disc was ˜8 mm, which is within the range for L4-L5 thickness reported in both males and females (Onishi, et al. (2019) Interdiscip. Neurosurg., 18:100523; Zhou, et al. (2000) Eur. Spine J., 9:242-248). Fiber-reinforced discs were die-cut using the same anatomical shape as the control discs. Fidelity of fit was checked by positioning the AVD between the 1:1 L4 and L5 replicate vertebrae.

### Compression Testing

Quasi-static compression tests of control, isotropic, and flocked AVDs were conducted on a uniaxial servo-hydraulic 810 material test system (MTS) employing a 25 kN loadframe which was calibrated by Instron (Instron, Norwood, MA). Load and displacement data for each AVD group were collected from the load frame and communicated through an 8800 minitower controller. The controller contains sensor conditioning cards for the system transducers and transfers data between the transducers and the computer. The controller also communicates with the load frame via a frame interface board (FIB) inside the load frame. The FIB links all the electrical components of the frame together. Stress relaxation and dynamic tests were performed on the AVDs to delineate differential mechanical properties. Subsequently, dynamic testing was performed at the end of a stress relaxation; this allows the sample material to be at equilibrium. In this experiment, the discs with an area of 1620 mm², were compressed to 20% strain for 180 seconds, relaxed for 600 seconds, and subjected to cyclic strain. Cyclic testing was conducted at a 1 Hz with a 2% strain amplitude for 100 cycles. Each experiment was repeated three times and similar results were gathered at room temperature (25° C.) and under ambient conditions. Aggregate data was collected and stored for further analysis. In most cases, AVDs returned to their original shapes after all compression tests. One sample from the flocked AVDs had minor delamination of a layer following compression cycles.

### Model Protein Release and Absorption Ratios

Absorption ratio was determined by taking the initial dry mass (mi) of NFY and MFs. After 24 hours of immersion in 1 ml of a phosphate buffered saline (PBS)/bovine serum albumin (BSA) (500 μg/ml), fibers were removed, lightly compressed to exude excess liquid, and weighed for a final wet mass (mf). A percent change in mass was calculated with the following equation:

Mass Change=(mf−mi)/mi×100%

Protein release was determined using BSA as a model protein and quantified with a BCA assay (Pierce BCA Protein Assay Kit, Thermoscientific, Rockford, IL). First, nine different dilutions for a standard curve using PBS (diluent) and BSA (stock) were prepared. Triplicates of approximately 0.2 g of NFYs and MFs were immersed in 1 ml of 500 μg/ml BSA in PBS and incubated at)(37° C. overnight. The following day, samples were moved into 1 ml of fresh PBS. After 5 minutes, 10 minutes, 15 minutes, 30 minutes, 1 hour, 2 hours, 4 hours, 12 hours, and 24 hours, 25 μl of the solution was removed and transferred into a 96 well plate. At the endpoint of the study, a mixture of BCA Assay working reagent was added and absorbance was measured at 540 nm using a plate reader (BioTek Synergy H2 Hybrid Reader, BioTek Instruments, Winooski, VT). Absorbance was correlated to BSA concentration using a standard curve established during reading.

### Cell Study

Prior to cell studies, salt-treated PCL MFs and chitosan/gelatin flocked scaffolds were fabricated, excised with an 8-mm biopsy punch, and sterilized with EtO gas sterilization for 24 hours. After gas sterilization, scaffolds were vigorously agitated in 70% ethanol for 24 hours to remove any loose fibers. Before seeding, scaffolds were immersed in completed DMEM. HaCaT cells were seeded at 10000 cells/scaffold and cultured for 7 days. At 3, 5, and 7 days, cells were stained with LIVE/DEAD™ staining kit and imaged using a confocal laser scanning microscope (Zeiss 710 Confocal Laser Scanning Microscope, Zeiss, Dublin, CA). Z-stack images were taken and used to construct both 3D and depth mapping renderings on Zeiss' Zen Blue software (Zeiss, Dublin, CA). Depth values are automatically generated by Zeiss' software and are presented unaltered. Viability was calculated as the fraction of live cells/total cells. After confocal images were acquired following live/dead staining, images were split by color (green and red) and cells expressing each color were counted using ImageJ's cell counter plugin. Normalized fluorescence was measured by reading the integrated density of the image and subtracting background fluorescence on ImageJ, as reported (Steele, et al. (2014) Biophys. J., 106:566-576). Cell migration was measured by taking length measurements from the depth of cells along the Z axis, resulting in a 3D length map of the imaged field.

### Animal Study

Ten-week old B6·BKS(D)-Leprdb/J male diabetic mice were purchased from Jackson Laboratory (Bar Harbor, ME) and were fed, housed, operated on, and cared for in-house at the University of Nebraska Medical Center. Two scaffolds were created: low-density (LF) and high-density (HF) flocked scaffolds consisting of PGLA (90:10) fibers (˜1 mm in length) and a 50/50 CHS/gelatin substrate/adhesive. Scaffolds were punched into 8 mm diameter scaffolds, washed with EtOH to remove loose fibers, and gas sterilized with ethylene oxide. Mice were anesthetized under 4% isoflurane in oxygen for roughly 5 minutes while on a heating pad. Diabetic status was ensured by taking blood glucose readings of each mouse. Each mouse was shaved and treated with buprenorphine SR (0.5-1 mg/kg body mass) injected subcutaneously. The surgical site was sterilized and treated thrice with povidone-iodine and EtOH wipes. During surgery, mice were maintained at 2% isoflurane. Two circular defects were made using a 8 mm sterile biopsy punch (Integra LifeSciences, Plainsboro Township, NJ), and a 10 mm inner diameter silicone wound splint was glued and sutured into place with silicone bonding adhesive and sutures. Splints were used to prevent dermal contraction, as several studies indicate the use of splinted wounds increases the mouse-to-human crossover (Masson-Meyers, et al. (2020) Int. J. Exp., 101:21-37). For treatment groups, each scaffold was directly inserted into the wound with the fibers entering the wound bed. Tegaderm was applied over each wound and post-operative monitoring for 30 minutes was conducted. Each group (LF, HF, negative control (no treatment)) had four mice with 2 wounds each. Two time points, 7 and 14 days, were used. Mice were housed for 7 and 14 days and at each time point 2 mice from each group were euthanized using CO₂ and bilateral thoracotomy. Explants of the wound and surrounding tissue were taken from each wound site and submerged in formalin for 1 week before undergoing hematoxylin and eosin (H&E) and Masson's trichrome staining (TRI) at the University of Nebraska Medical Center's in-house tissue science facility. Histology images were analyzed, and the most representative images were presented. Re-epithelialization was measured as the percent of new epithelial layer over the wound area. Angiogenesis was measured as the number of vessels/mm² and vessels were identified with two criteria: 1) presence of endothelial lining and 2) presence of erythrocytes. To measure granulation tissue formation, collagen deposition was indirectly measured using integrated density (the sum of the values of the pixels within the region of interest (ROI)) (Chen, et al. (2017) Int. J. Clin. Exp. Med. 10:14904-14910). Briefly, slides stained with TRI were opened in ImageJ. Within the wound area (defined within the 8 mm defect size), images underwent color deconvolution. Green color channels omit stained cellular nuclei and retain collagen. The integrated density was subsequently measured within each ROI (integrated density=ROI area×mean gray value).

### Statistical Analysis

Other than the cyclic compressive data for AVDs, all data is expressed as the mean±standard deviation and a minimum of 3 samples were used in each experiment. Mechanical data from AVD testing was expressed as median values. For pairwise columnar comparisons, ordinary one-way ANOVA with Tukey's multiple comparisons post hoc testing were performed. For grouped analyses, 2-way ANOVA with Tukey's multiple comparisons post hoc testing was performed. Curve fitting followed GraphPad Prism's curve fitting guide. Line plots (e.g., stress-strain, force-strain, and distribution) curves underwent 10% smoothing. All measurements taken on SEM or photograph images used calibrated length measurements. Statistical significance was expressed as *p<0.05, ** p<0.01, *** p<0.001, **** p<0.0001, and a lack of pairwise comparison bars implies a lack of significance (p>0.05). Figures are all original and were created using BioRender and Microsoft PowerPoint. All statistical analyses were performed using GraphPad Prism 9 for macOS (version 9.1.1)

### Results

MFs and NFYs Make Size-Tunable Flock Fibers with Distinct Functional Properties

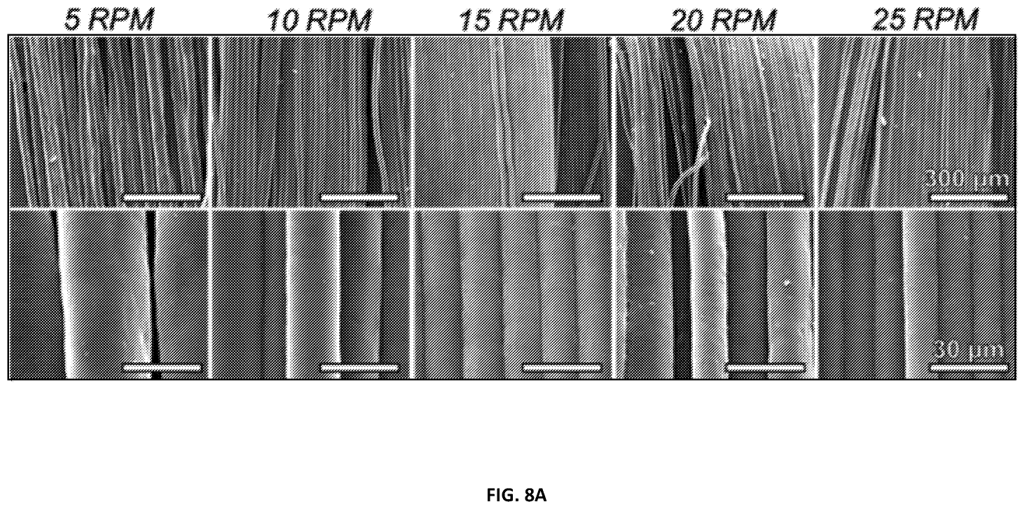

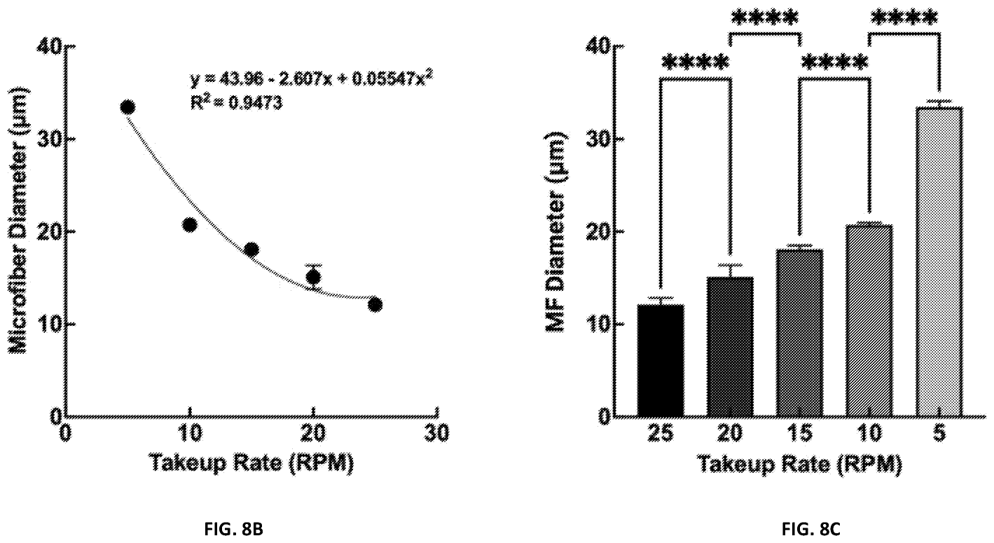

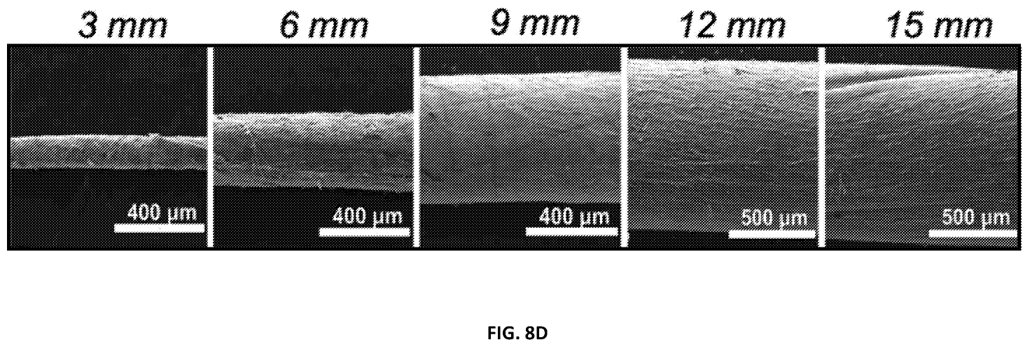

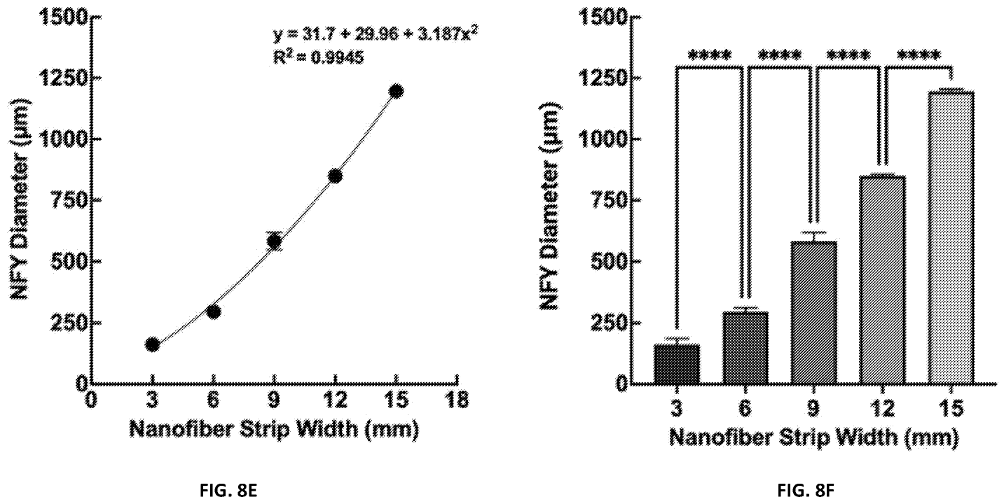

Fiber morphologies and topographies effect cell migration rates and lineage differentiation (Gossla, et al. (2016) Acta Biomater. 44:267-276; Walther, et al. (2012) Materials 5:540-557). The ability to tune the size and topography of fibers for tissue engineering applications offers a variety of increased tunability, particularly in cases where it may be favorable to adjust the fiber surface area to increase or decrease the migratory rate of localized cells (Hwang, et al. (2009) Biomed. Microdevices 11:739-746). To this end, PCL MFs produced from wet spinning were tuned and standardized with a best-fit trend line. The diameters of the fibers were adjusted by increasing the rotations per minute (RPM) of the take-up drum during wet spinning. As take-up rate increased, the average fiber diameter decreased. At 5, 10, 15, 20, and 25 RPMs, PCL fiber diameters were 33.45±0.65, 20.74±0.24, 18.07±0.44, 15.10±1.3, and 12.11±0.72 μm, respectively (FIGS. 8A-8C). Diameters varied significantly at each 5 RPM interval change (P<0.0001, n=100). Similarly, NFYs prepared from twisting electrospun nanofiber have easily-controlled diameters which are determined by the initial width and thickness of the nanofiber mat (Chen, et al. (2017) Nanomedicine 12:2597-2609). Nanofiber mats approximately 30 μm thick were cut into 3, 6, 9, 12, and 15 mm strips and consisted of semi-aligned nanofibers as confirmed via SEM imaging. Most nanofibers diameters ranged from 500 nm to 1 μm. The nanofiber strips were rolled along the length of the collection drum to form NFYs. As anticipated, thinner strips resulted in NFYs with smaller diameters. Strips 3, 6, 9, 12, and 15 mm in width resulted in NFYs with 161.5±24.70, 295.8±15.59, 583.40±35.50, 848.8±6.237, 1195±8.854 μm in diameter (P<0.0001, n=100).

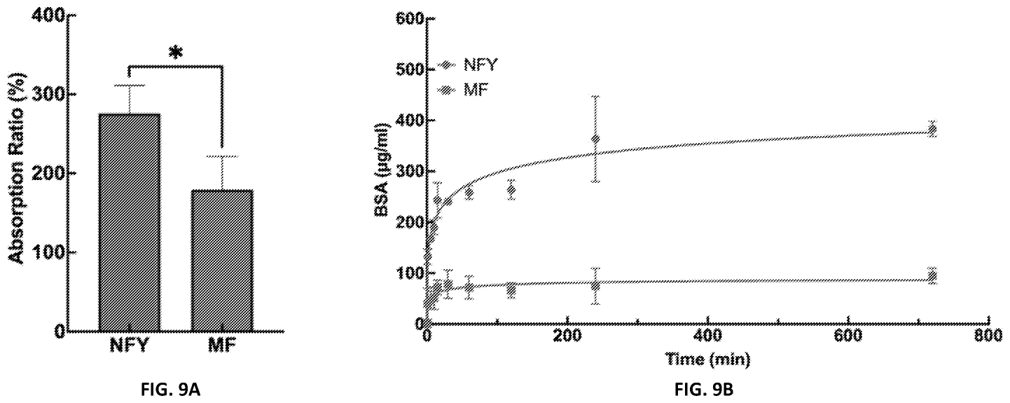

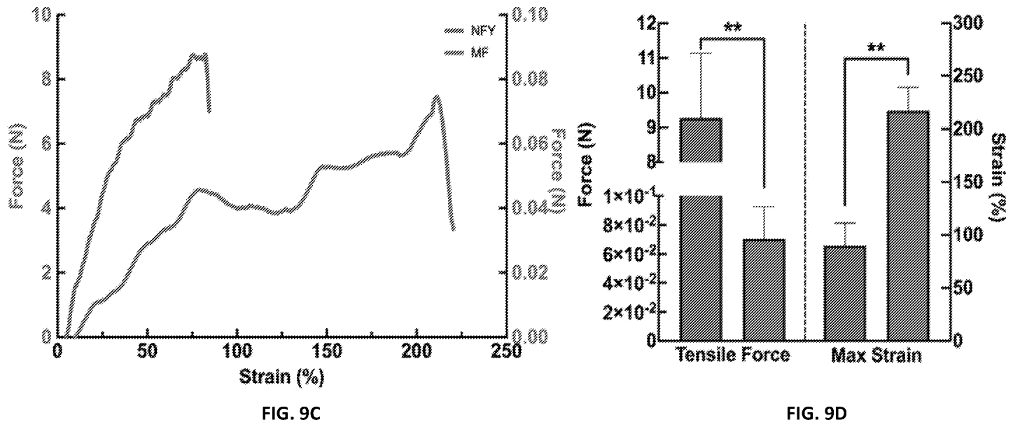

Due to their dramatically different bulk and surface properties, MFs and NFYs vary in functionality and mechanical stability. To demonstrate how each type of fiber has distinct properties, a series of mechanical tests and protein absorption/release studies were conducted. An equivalent mass (2 mg) of MFs and NFYs were totally submerged in a BSA solution (500 μg/ml) for 10 minutes and their absorptive properties were evaluated. NFYs absorbed significantly more (276.00±35.29)% of their mass than MFs (179.40±42.08)% (P=0.0382, n=6) (FIG. 9A). Similarly, a BSA release assay was carried out after allowing each fiber type to reach maximum saturation. Protein elution was measured by incubating 2 mg of each BSA-saturated fiber in 37° C. PBS solution for 12 hours. At every collection time point (1, 5, 10, 15, 30, 60, 120, 240, and 720 minutes), NFYs released significantly more protein than the equivalent mass of MFs (FIG. 9B) Additionally, after 60 minutes, MFs had no significant increase in BSA release, while NFYs had continued significant BSA release up to 240 hours. By the conclusion of the BSA release assay, NFYs achieved a total BSA concentration of nearly 400 μg/ml, while MFs achieved just under 100 μg/ml. Fundamental differences in fiber morphologies (i.e., NFYs have microporous surface properties while MFs are smooth and solid) dictate their absorption and release abilities (Maleki, et al. (2016) Biofabrication 8:035019; Sohrabi, et al. (2013) Polymer 54:2699-2705). MFs rely upon adsorption (surface attraction) to hold and release compounds, while NFYs can absorb solutions between and within their nanofibrous pores (Paulose, et al. (2010) Soft Matter 6:2421-2434). The degree to which NFYs are spun and their original nanofiber composition can be used to tune their release profiles, whereas MFs are limited to their bulk composition and morphology.

In addition to having distinct absorption and release profiles enabled by differences in morphological features, MFs and NFYs exhibit different mechanical properties as well. For mechanical tests, MFs and NFYs with average diameters 33.45 μm and 161.5 μm were subjected to tensile testing. Monofilament MFs exhibited higher elasticity, reaching a maximum strain of (217.00±21.98)%, but a lower break force averaging 0.07±0.022 N. Conversely, NFYs had a significantly lower max strain of (89.95±21.38)%, but a higher break force of 9.267±1.87 N (P=0.002 and P=0.001, respectively, n=3) (FIGS. 9C-9D). Fiber diameter and bulk morphological composition undoubtedly have large roles in modulating mechanical features of the fibers. In this case, NFYs are stronger than MFs due to their larger size and nanofibrous multifilament composition (Liu, et al. (2021) Mater. Lett. 300:130229), though they are not as elastic as they resist fiber necking more than MFs (Verschatse, et al. (2020) Polymers 12:2581). Crystallinity and polymer chain orientation have been examined as primary drivers behind mechanical strength of fibers (Brennan, et al. (2018) Adv. Healthc. Mater. 7:1701277). Though the NFYs and MFs have different cross-sectional surface areas, the multifilament and twisted nanofibers within the NFYs do not allow for as much linear displacement and impart the majority of the tensile strength noted.

### Reversible Incorporation of NaCl Improves Flock Yields

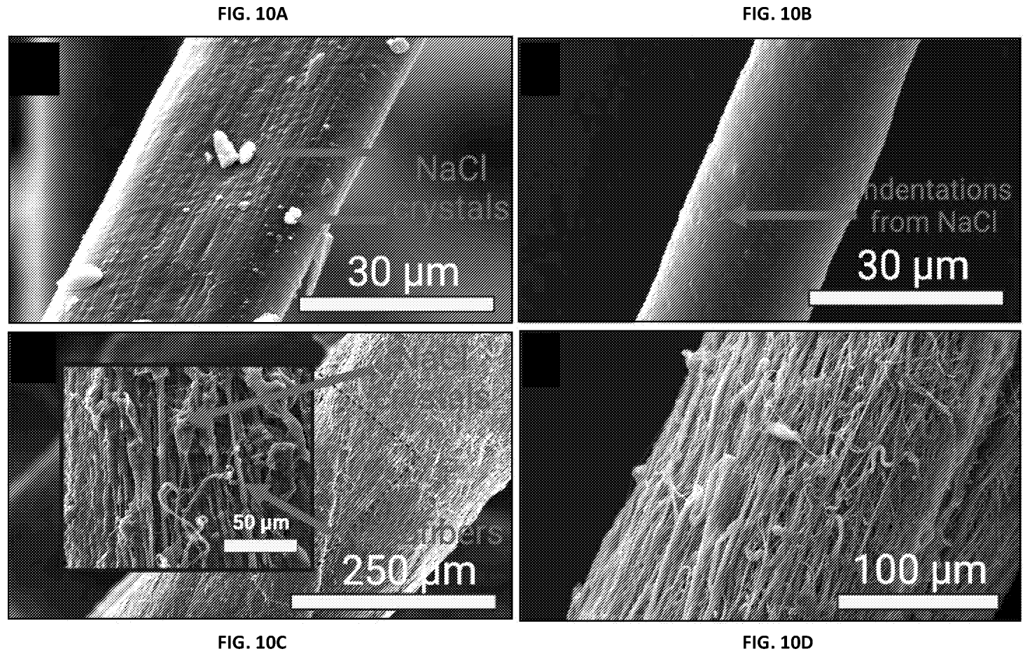

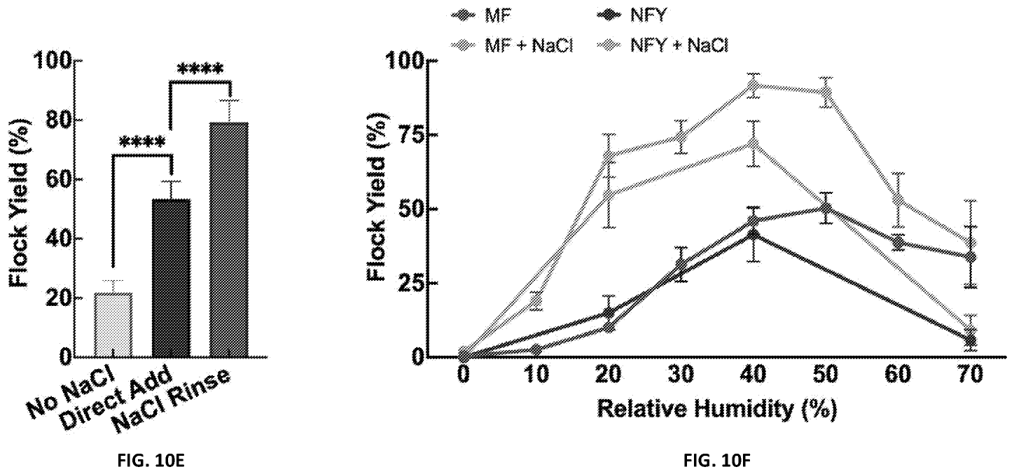

To create surfaces capable of accumulating sufficient charge for flocking, a novel method utilizing NaCl (salt) was developed. The rationale behind using NaCl as an ionizable salt-based surface treatment was based on its in situ tolerability at modest concentrations, ease of use, low cost, and removability (Kiran, et al. (2012) J. Non-Cryst. Solids 358:3205-3211; Russo, et al. (2015) Soft Robot. 2:146-154; Chayad, et al. (2016) J. Eng. Technol. 34:1265-1274; Zhao, et al. (2018) Adv. Mater. 30:1800598,). During hydration, NaCl crystals dissociate into their ionic forms Na+ and Cl−. The incorporation of ionic liquids onto the surface of the fibers allows charge accumulation to occur and a charge differential is maintained until a sufficient Coulombic force lifts the fibers towards the ground electrode (Xia, et al. (2009) Macromolecules 42:4141-4147; Ouyang, et al. (2004) Polymer 45:8443-8). To this end, two methods for incorporating salt onto MFs were tested for efficiency at RH=40%. In one case, fine salt was directly added to the polymer solution prior to wet spinning or electrospinning and, in the other case, a saturated salt solution was used during the final rinse of the fibers before flocking. The presence of salt was confirmed via SEM, in which salt crystals appeared on the surface of both MFs and NFYs (FIGS. 10A, 10C). The salt crystals could be easily removed by agitating the flocked scaffolds in a water bath for several seconds, which was confirmed by SEM following a water rinse (FIGS. 10B, 10D). To evaluate which salt incorporation technique resulted in the highest flocking yield, a known mass of MFs from each method were flocked. MFs with salt directly added during fabrication had an average flock yield of (53.33±6.02)%, MFs with a salt wash prior to flocking had an average flock yield of (79.17±7.30)%, and untreated MFs had an average yield of (21.82±4.12)% (P<0.0001, n=6) (FIG. 10E).

After determining whether a salt wash prior to flocking resulted in the highest yield, the effect of RH on flock yield was elucidated. MFs and NFYs with and without salt treatment were flocked at different RHs. As previously mentioned, hydration on the fiber surfaces is necessary for ionization of the NaCl crystals to occur. A fine interplay between ionizing the surface of the fibers and weighing them down with overhydration exists. This system was optimized by flocking salt-treated MFs and NFYs at different RH. Salt-treated MFs achieved a maximum flock yield of (91.67±4.04)% at RH=40%, while untreated MFs achieved a maximum flock yield of (50.33±5.13)% at RH=50%. From RH 60-70%, fibers experienced a decrease in flock yield. Between RH 10-50, salt-treated MFs had significantly higher flock yields compared to untreated MFs (P</=(0.0001-0.0214), n=3) (FIG. 10F). Similarly, NFYs achieved a maximum flock yield of (72.09±7.64)% at RH=40%, while untreated NFYs achieved a maximum flock yield of only (41.42±9.10)% (P<0.0001, n=3). With increasing RH, NFYs experienced a more rapid decrease in flock yield, likely due to their ability to absorb water, and thus increasing their mass. Flock yields of each fiber type at each RH are shown in Table 2. Increasing mass necessitates a stronger Coulombic force for flocking, and salt ionization on the surface of NFYs is limited to mostly their surface. Therefore, achieving a RH that facilitates total surface wetting is necessary for uniform charge accumulation. Despite their significantly larger size and mass, one advantage NFYs have over MF during flocking is their reduction in fiber-fiber interactions. After sieving MFs, it is common to have some fibers adhere to one another, particularly at higher RHs. This phenomenon is common and tends to increase with decreasing fiber size. During flight, these fiber aggregates can interfere with flock uniformity and yield and are referred to as clouding. Nevertheless, in both cases, MFs and NFYs exhibited an increased flocking yield after a salt treatment and suffered from decreased flock yields at high RHs. Based on these findings, fibers should be stored and flocked between RH 20-50%. After scaffolds are cured, the salt on the surface of the fibers can be removed, as high concentrations of salt may induce local apoptosis by increasing the expression of heat shock protein 60 (HSP60) (Jakic, et al. (2017) PLOS One 12:e0179383).

TABLE 2
Flocking yields at different relative humidities (RH).

| RH (%) | MF | MF + NaCl | NFY | NFY + NaCl |
|---|---|---|---|---|
| 0 | 0.33 ± 0.58 | 2.00 ± 0.99 | 0.00 ± 0.00 | 0.00 ± 0.00 |
| 10 | 2.57 ± 0.91 | 19.01 ± 3.00 | | |
| 20 | 10.10 ± 1.72 | 68.00 ± 7.21 | 15.03 ± 5.78 | 54.77 ± 11.02 |
| 30 | 31.30 ± 5.7 | 74.33 ± 5.50 | | |
| 40 | 46.00 ± 4.58 | 91.67 ± 4.04 | 41.42 ± 9.10 | 72.09 ± 7.64 |
| 50 | 50.33 ± 5.13 | 89.33 ± 5.03 | | |
| 60 | 38.79 ± 2.57 | 53.00 ± 9.00 | | |
| 70 | 33.87 ± 10.28 | 38.67 ± 14.19 | 5.78 ± 3.54 | 9.22 ± 5.10 |

MF: microfiber; MF + NaCl: microfiber treated with NaCl; NFY: nanofiber yarn; NFY + NaCl: nanofiber yarn treated with NaCl.

### Characterization of Flocked Microfiber and Nanofiber Yarn Scaffolds

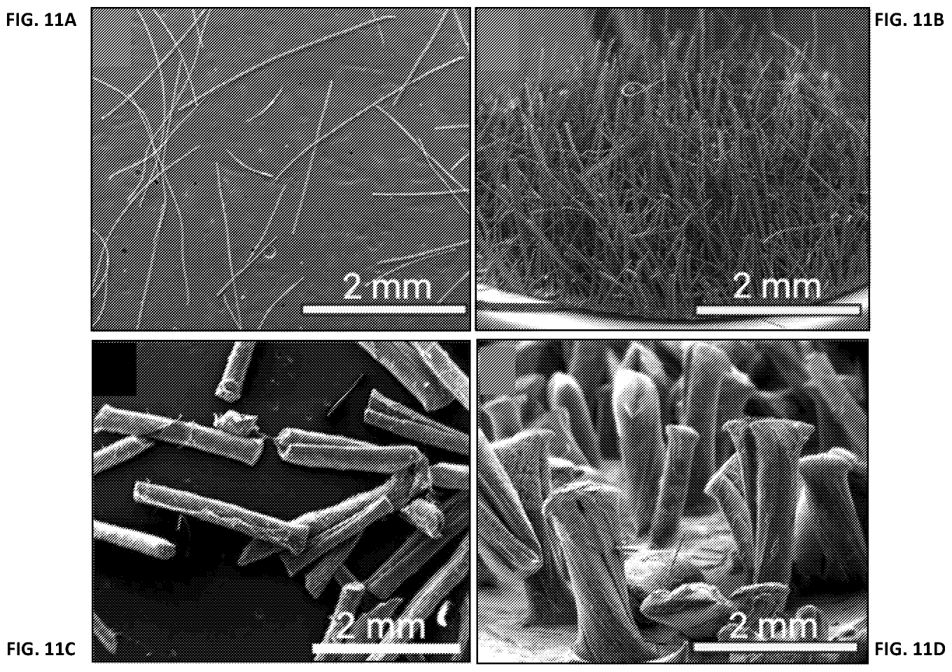

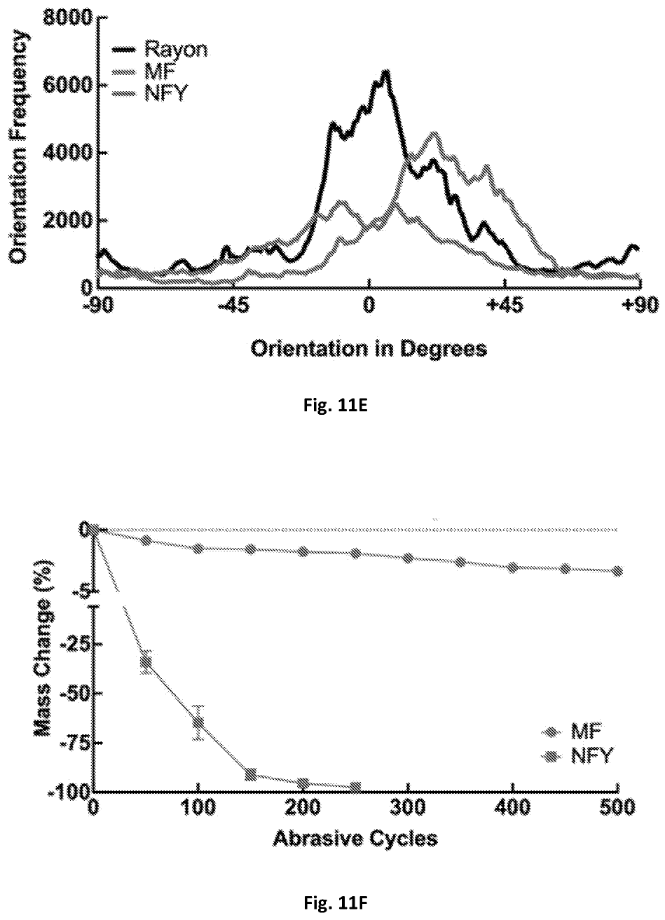

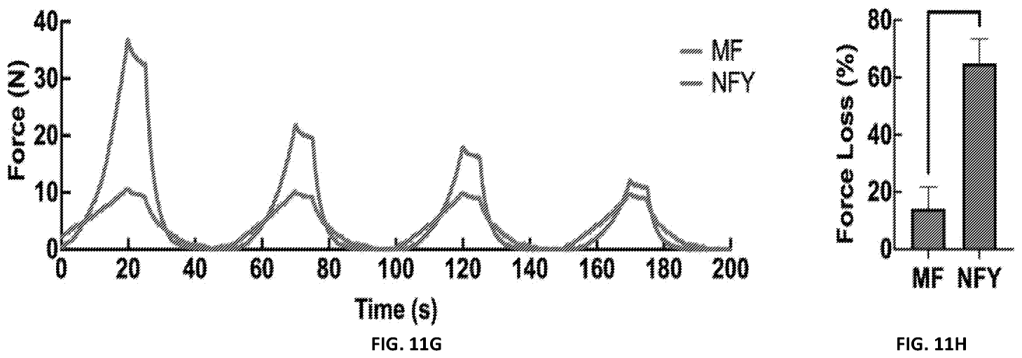

Some characteristics of electrostatically flocked scaffolds include: 1) fiber density and thus porosity is mainly modulated by flock time (i.e., how long voltage is applied) and fiber amount sieved (but not fiber length); 2) scaffolds with shorter fibers and higher fiber densities have higher compressive moduli; and 3) CHS, gelatin, and CHS/gelatin adhesive/substrates are mechanically suitable for in situ and in vivo applications. Both MFs and NFYs ranged in length from 1 to 2.2 mm in length, as verified by SEM imaging in FIGS. 11A and 11C. Flocked scaffolds consisting of MFs demonstrated a velvety appearance, while scaffolds composed of NFYs appeared to be more sparsely populated (FIGS. 11B, 11D). Orientation analysis comparing MFs, NFYs, and rayon (industry control) showed that rayon fibers had the highest degree of alignment, though MF scaffolds had similar orientation distributions (FIG. 11E). NFYs had relatively broad distribution peaks, in part due to the dramatic size variation and relative impact on colorimetric tensor measurement. Abrasion resistance revealed that MF scaffolds lost less than 5% of their mass after 500 abrasive cycles, while NFY scaffolds lost nearly 100% by 250 cycles (FIG. 11F). Due to the dogbone shaped ends on the NFYs, it is reasonable to assume the amount of NFYs embedding into the adhesive layer is less than that of MFs. In addition, NFYs have larger surface area and lower elasticity so they resist abrasion more than MFs, resulting in a faster mass loss during abrasive cycles. Evaluation of compressive strength of MF and NFY scaffolds showed distinctly different compression curves. After 4 cycles of 50% displacement, compression curves were normalized and compared (FIG. 11G). Notably, NFYs had a 3-fold higher max compression resistance (36.95 N) during the first compressive load compared to MF scaffolds (10.66 N). However, the compression resistance decreased significantly between the successive loading periods, due to the permanent deformation caused by NFYs being flattened. Unlike the NFY scaffolds, MF scaffolds had little change in compression resistance between the first and last compression. The force change between the fourth and first load was calculated for each scaffold and expressed as a percent loss. NFY scaffolds lost (64.95±8.42)% of their maximum compressive load, while MF scaffolds lost only (14.26±7.51)% (P=0.0015, n=3) (FIG. 11H). Based on these results, MF scaffolds were used for the remaining in vitro and in vivo studies.

### Flocked Scaffolds Sustain and Modulate Cellular Proliferation

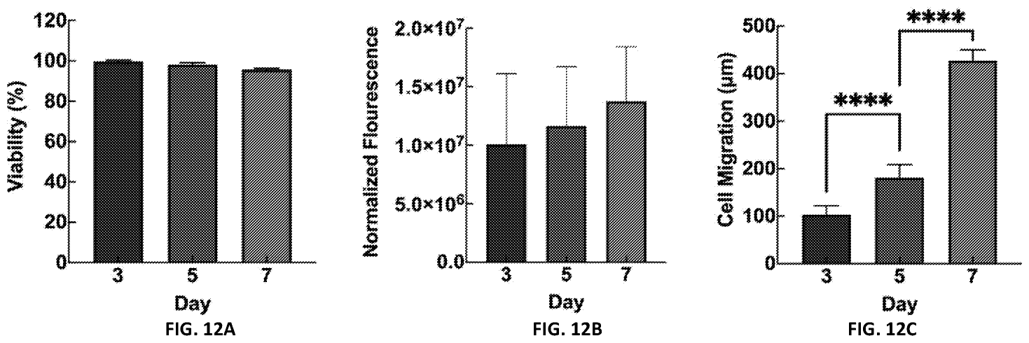

On their own, flocked scaffolds have demonstrated the ability to modulate cell migration, sustain tissue formation, and facilitate cell differentiation. Confocal images including brightfield reveal densely populated scaffolds at days 3, 5, and 7. By day 7, HaCaTs appeared to form larger cell aggregates, presumably along fibers. 3D mapping of the cells allows for spatial orientation to be observed as well as confirmation that fibers retain anisotropy under culture conditions. It is worth noting that the fibers retain alignment under culture conditions, but that this is largely due to how well the fibers embed into the adhesive and how inherently rigid the fibers are. To this end, cells appeared relatively homogeneously distributed along the scaffold fibers. To quantify the cell migration, the height of cells along fibers were quantified with colorimetric depth mapping. Analysis of the Live/Dead, 3D Live/Dead distribution, and depth mapping images revealed several key findings. First, color channel splitting of LIVE/DEAD staining (e.g., red, and green channels) revealed excellent viability over the 7-day culture period, with days 3, 5, and 7 achieving viabilities of (99.7±0.58)%, (98.00±0.97)%, and (95.67±0.61)%, respectively (FIG. 12A) (P=0.0754, n=3). Given that a relatively high density of cells was seeded, the slight decrease in viability is a result from cells detaching after reaching confluency. Similarly, the normalized fluorescence intensity of the 3D Live/Dead, measured as normalized integrated density throughout the entirety of the scaffold volume, revealed a steady increase in intensity throughout the 7-day culture, though large local deviations in 3D intensity were noted where cells did not occupy the scaffold (FIG. 12B) (P=0.6244, n=3). Finally, spatial analysis from depth mapping revealed significant migration was achieved at each time interval, with cells traveling an average of 103.20±18.27 μm, 181.11±27.33 μm, and 427.10±22.75 μm at day 3, 5, and 7, respectively (FIG. 12C) (P<0.0001, n=3).

### Flocked Scaffolds Show a Fiber Density-Dependent Wound Healing Effect

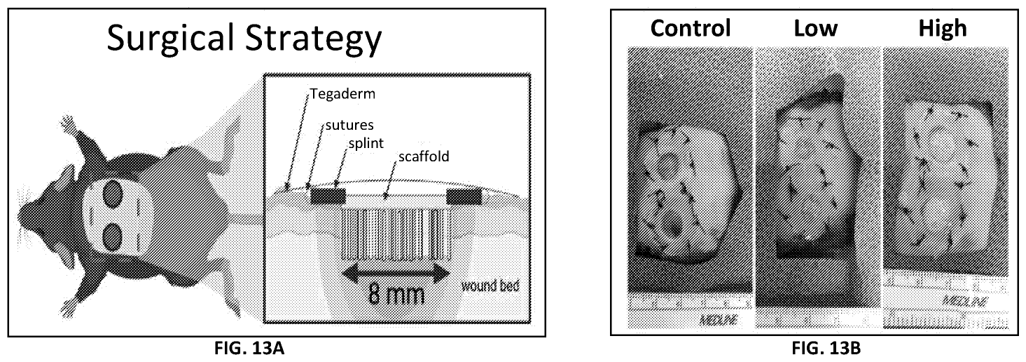

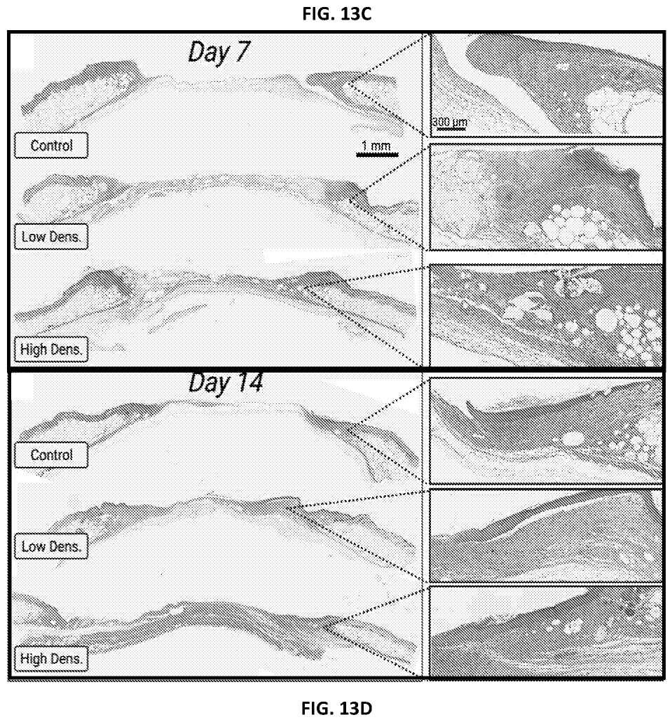

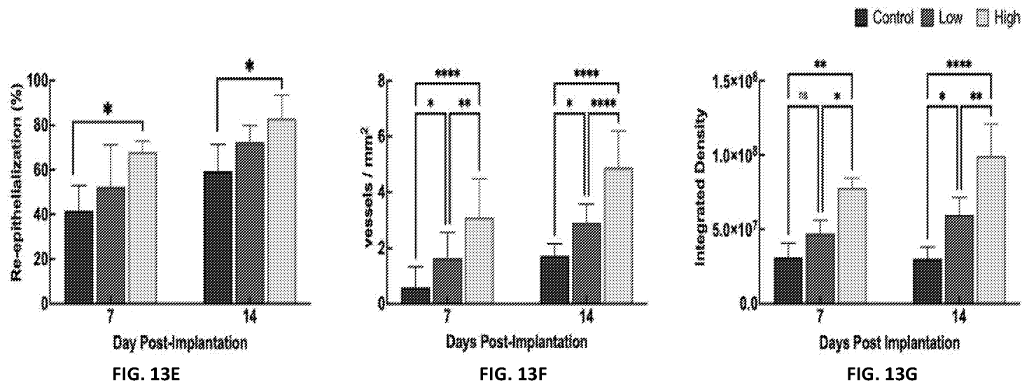

To evaluate the wound healing capacity of rapidly-degrading flocked PGLA (90:10) fiber scaffolds with CHS/gelatin adhesive as substrate, scaffolds were placed directly in contact with 8 mm induced full thickness wounds in type II diabetic mice and observed for 7 and 14 days. Scaffolds were positioned with fibers penetrating into the wound bed such that the substrate was near flush with the epidermis on the wound edge (FIG. 13A, 13B). Several metrics were measured as wound healing hallmarks following histological analysis of H&E and TRI staining at day 7 and 14 post-implantation (FIGS. 13C, 13D). Re-epithelialization, measured as the percent of the original defect that was reepithelialized by day 7 and 14, showed a fiber-dependent, wound healing effect. By day 7, mice without treatment reached (41.53±11.43)%, while those with low-density and high-density flocked scaffolds reached (52.37±18.90)% and (68.00±4.88)%, respectively (P=0.013, n=2) (FIG. 13E). Similarly, after 14 days, mice without treatment reached an average re-epithelialization of (59.40±12.07)%, and those with low-density and high-density flocked fibers reached (72.30±7.7)% and (83.02±10.50)%, respectively (P=0.0277, n=2) (FIG. 13E). In addition to modulating re-epithelialization, blood vessels within the region of interest (the wound edges and within the defect area) were quantified for each treatment group at each timepoint. Vessel counts at day 7 were 0.58±0.24, 1.64±0.92, and 3.09±1.4 vessels/mm² for control and low-density and high-density flock scaffolds. Similarly, vessel counts at day 14 were 1.72±0.43, 2.90±0.69, and 4.89±1.30 vessels/mm² for control and low-density and high-density flock scaffolds. Both between and within group significance was present at each time point (P<0.0001, n=12) (FIG. 13F). From day 7-14, the high-density flock scaffold group experienced the most significant increase in vessel density (P<0.0001, n=4), followed by low-density flocked scaffolds (P=0.0069, n=4) and control groups (P=0.017, n=4). There are several explanations for the fiber-dependent angiogenesis observed, and a synergistic mechanism may be at play. First, the number of fibers within the wound decreases between days 7 and 14, which was the desired and expected outcome when using PGLA (90:10). PGLA has a notably fast degradation rate due to the smaller ratio of lactic acid (which increases hydrophobicity of the methyl group on the lactide monomer), and products of degradation are lactic and glycolic acid (Makadia, et al. (2011) Polymers 3:1377-1397). Studies have indicated a positive relationship between lactic acid and angiogenesis/wound healing, which can explain the fiber density-dependent angiogenic effect observed (Porporato, et al. (2012) Angiogenesis 4:581-592). Herein, sustained local lactate release from biodegradation of PLA and PLGA lead to reparative angiogenesis in superficial and ischemic wounds, and a similar effect may be at synergistic play in this scenario. In addition, mechanical perturbations and microstresses induced by the penetration and/or presence of flock fibers in the wound may trigger fiber density-dependent angiogenesis (Liebl, et al. (2012) J. Am. Coll. Clin. Wound Spec. 4:2-6; Sewell-Loftin, et al. (2020) Lab Chip 20:2776-2787). Granulation tissue formation, a hallmark in wound healing, is often subjectively measured (Wang, et al. (2016) Acta Biomater. 30:246-257). In this case, color deconvolution of TRI staining within the wound bed were used to quantify collagen deposition as a proxy measure of granulation tissue formation (Jorgensen (2003) APMIS Suppl. 115:1-56; Cremers, et al. (2015) Front. Med 2:86). By day 7, control, low-density and high-density flock scaffolds reached integrated density values of (3.12±0.93)×10⁷, (4.72±0.90)×10⁷, and (7.80±0.65)×10⁷ a.u., respectively (P<0.0014, n 3). By day 14, integrated density values achieved (3.03±0.76)×10⁷, (5.97±1.17)×10⁷, and (9.93±2.14)×10⁷ a.u., respectively (P<0.0001, n=3) (FIG. 13G). Another noteworthy observation is the decrease in fiber remnants between days 7 and 14 in all groups, indicating the PGLA (90:10) fibers have suitable degradation profiles in vivo for wound healing. Daily gross anatomical examination revealed no superfluous production of wound exudate, changes in animal behavior, or noticeably different degrees of inflammation. Further, gross anatomical examination found that many fibers retained alignment in situ with tissue having formed around them. No significant differences in masses were noted between or within groups (P=0.7627, n=4).

### Flock Fiber-Reinforced Artificial Vertebral Discs are Mechanically Robust

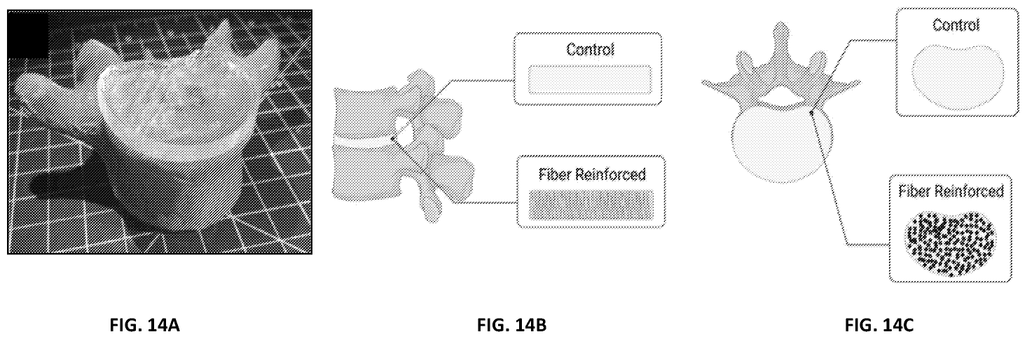

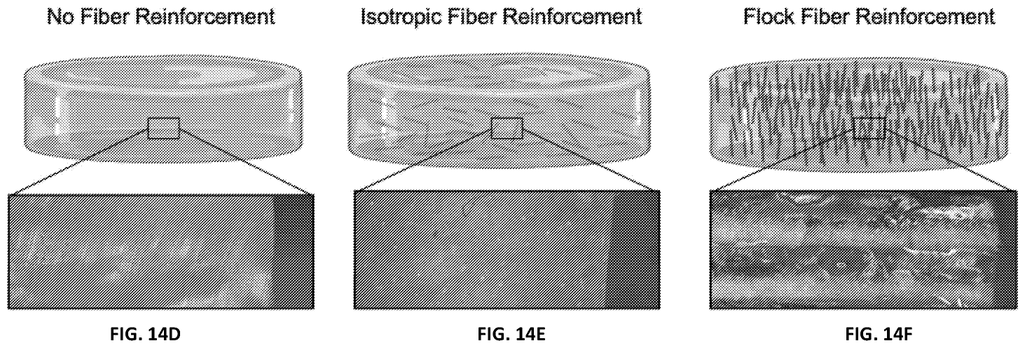

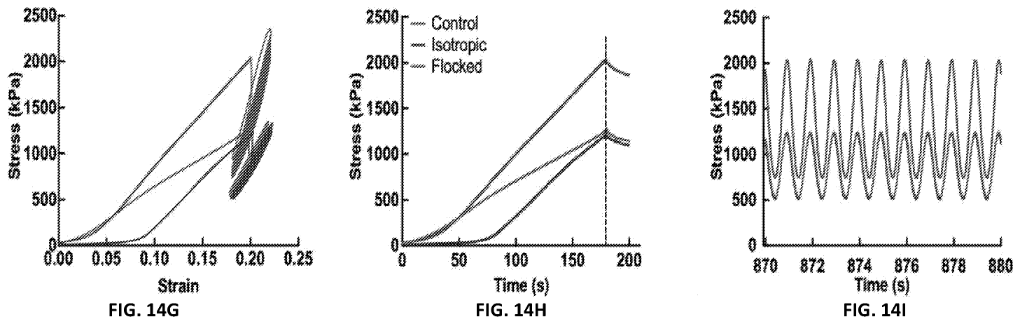

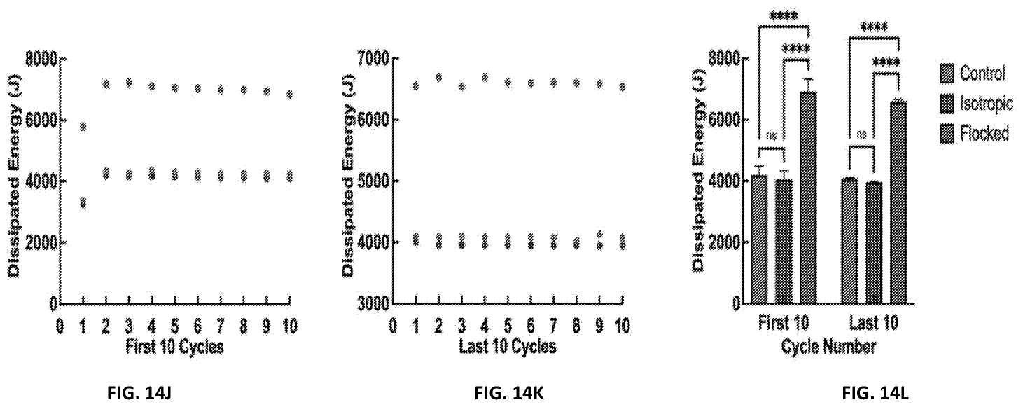

To demonstrate how flocking may be used in a stress-shielding manner, a proof-of-concept AVD substitute utilizing flocked layers to emulate the angle-ply structure of the native disc was created using PLA flock reinforcement. AVDs were engineered based on anatomically accurate vertebrae (FIG. 14A) with the goal of reinforcing elastomeric disc replacements (FIGS. 14B, 14C), which are one modality for disc replacement (Pimenta, et al. (2010) SAS J 4:16-25). Pure PDMS (FIG. 14D), isotropic (FIG. 14E), and flocked (FIG. 14F) discs were evaluated for several hallmark metrics as AVDs. Preliminary tests showed that all discs exhibited elastomeric behavior, returning to their original shape after compression, unless fracture occurred. Stress relaxation and dynamic tests were performed on the discs to characterize the difference in performance between pure elastomeric discs, isotropic reinforced discs, and flocked discs. Comprehensive stress-strain curves during loading and cyclic compression are presented in FIG. 14G. The relaxation period was deemed sufficient because the equilibrium modulus had a variation of 0.1% or less in the last 10 s before the start of the cycles (Table 3). For each sample, the equilibrium modulus was calculated by dividing the stress at equilibrium by the strain at equilibrium (Patel, et al. (2019) Tissue Eng. Part C Methods 25:593-608). The equilibrium modulus of the flocked (8017.8 kPa) disc at the end of the relaxation was almost double the modulus of the isotropic (4459.7 kPa) and the control (4649.6 kPa) disc. During loading, flocked discs required approximately 75% more load (2051 kPa) than the control (1290 kPa) and the isotropic (1226 kPa) discs to reach 20% strain (FIG. 14H). The stress amplitude during the cyclic loading was the highest for the flocked disc. Upon ramping, the flocked and isotropic discs exhibited a higher stiffness as compared to the control disc, as evident by their steeper loading curves. Though the control and the isotropic discs exhibited the same deformation and stress response at 20% strain and during cyclic loading, the flocked discs showed a higher stiffness only after 10% of the strain during the loading phase. This observation is supported by the strain energy in Table 3. The strain energy of the flocked discs (179.2 J) was more than twice the strain energy of the isotropic (80.5 J) and control (124.7 J) discs. The layer reinforcement in the flocked discs increased the resistance to deformation and isotropic strain after 10% strain. During the cyclic loading, all the samples exhibited the same phase lag response (FIG. 14I). While the isotropic and the control discs exhibited the same stress response, the flocked discs considerably varied in stress amplitude within the 1% strain of cyclic loading. This observation is explained by the dissipated energy of the discs in the last 10 cycles. The dissipated energy is the area in the middle of the hysteresis loop. All samples had increasing stress dissipation during the first 10 cycles (FIG. 14J), but it was observed that the flocked disc dissipated nearly twice the energy of the isotropic and control discs in the first and last 10 compressive cycles (FIG. 14K, 14L) (P<0.0001, n=10). The consistency in dissipated energy in the last 10 cycles is an indicator that the hysteresis loops were aligned and there was minimal deviation in loading and unloading (FIG. 14G). The anisotropy imparted by the flocked layers contribute to the increased stiffness and compressive strength, though the mechanical characteristics of the PDMS (i.e., hysteresis looping and elastomeric behavior) are retained. In addition to the anisotropy of the flocked fibers, the use of layered flocked structures contributes to the bulk material toughness. In this case, the choice of an elastomer as the matrix may increase the durability of the AVD, which is a critically important consideration in designing AVDs. Flock fiber reinforcement does not change the shape or surface finish of the elastomeric AVD and as a platform, could be built upon to create biologically-relevant flock-reinforced elastomer biomaterials for AVDs or other load-bearing applications.

TABLE 3
Mechanical characteristics of elastomeric disc replacements.

| | Equilibrium | Delta | Strain |
|---|---|---|---|
| | modulus | Equilibrium | Energy |
| Model | (kPa) | (% last 10 s) | (J) |
| Control disc | 4649.6 | 0.07 | 124.7 |
| Isotropic disc | 4459.7 | 0.09 | 80.5 |
| Flocked disc | 8017.8 | 0.1 | 179.2 |

The present study demonstrates several important proof-of-concept studies related to electrostatic flocking. First, a salt (NaCl)-based system for increasing surface charge accumulation on inherently electrostatically insulative polymer fibers is demonstrated. Conventional electrostatic flocking relies upon the ability of flocking fibers to accumulate sufficient charge for flocking. Here, simple salt ionization is shown to be an effective alternative approach for charge accumulation. Because the salt treatment is confined to surface adhesion, simply washing the flocked objects with water allows for rapid and effective removal of salt residues. This methodology enables flocking of intrinsically-insulative polymers without utilization of cytotoxic or proprietary organic salt and aliphatic acid finishes. Second, for the first time, the electrostatic flocking of NFYs fabricated by rolling electrospun nanofiber strips is shown. NFYs are mechanically robust, capable of flocking in quantities similar to MFs, and capable of releasing absorbed molecules. This allows for constructing flocked scaffolds with enhanced functionality via incorporation of drugs and other biologics. To highlight biomedical applications of electrostatic flocking, HaCaT cells were cultured on scaffolds and the ability to modulate cell migration along the length of the fibers was demonstrated. To apply this towards a clinically relevant in vivo model, the PCL fibers were replaced with fast degrading PGLA (90:10) fibers and it was investigated as a wound treatment in a diabetic mouse model. The results from the wound healing study affirmed the degradation of fibers and reaffirmed a fiber density-dependent effect with improvement in wound healing. Fibers induced granulation tissue formation and increased re-epithelization. Finally, to demonstrate how flocking can be used as a functional technique for inducing structural anisotropy, a proof-of-concept, anatomically-accurate L4-L5 AVD was synthesized. AVDs with flocked layers required 75% more load to achieve 20% strain than control and isotropic AVDs. Similarly, flocked AVDs dissipated more energy, had a higher equilibrium modulus, and retained elastomeric shape recovery after 100 supraphysiological compressive loads. Taken together, this study highlights several clinically relevant applications of a simple yet impactful flocking methodology.

### Example 3

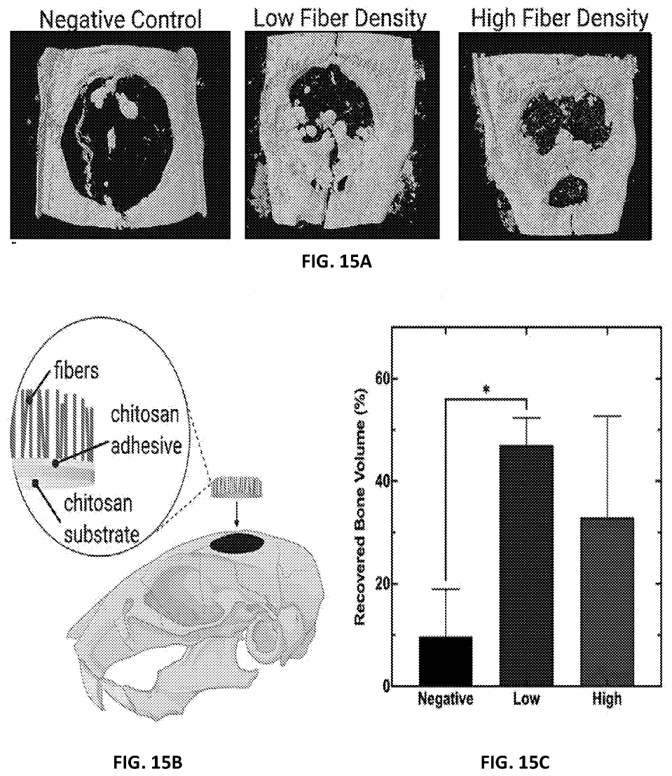

The bone regenerative capabilities of 0.5% AgNP/PCL fiber and chitosan adhesive/substrate scaffolds was studied in an 8-mm rat cranial defect model. Flocked scaffolds were custom prepared for each animal model to fit an 8 mm surgically induced total cranial bone defect. During surgery, the scaffold substrate was laid flush against the in-tact dura mater, with the fibers filling the empty volume of the defect (FIG. 15B). The periosteum was sutured along the top of the scaffold fibers. Micro computed tomography images show that, in both flock scaffold treatment groups, total bridging of the defect was achieved, while no significant bone volume was regenerated in the negative control (FIG. 15A). These results demonstrated a fiber-dependent and significant recovery of bone volume over a 7-week period compared to a negative control (FIG. 15C).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A flocked substrate comprising microfibers attached to a substrate, wherein said microfibers comprise a polymer and a conductive filler.

2: The flocked substrate of claim 1, wherein said microfibers are substantially perpendicular to the substrate.

3: The flocked substrate of claim 1, wherein the density of microfibers on the surface of the substrate is greater than about 50 fibers/mm².

4: The flocked substrate of claim 1, wherein a surface of said substrate is coated with an adhesive.

5: The flocked substrate of claim 1, wherein said microfibers have a length of about 200 μm to about 5 mm.

6: The flocked substrate of claim 1, wherein said polymer is an insulative polymer.

7: The flocked substrate of claim 6, wherein said polymer comprises polycaprolactone (PCL), poly (lactide) (PLA), and/or poly (lactic-co-glycolic acid) (PLGA).

8: The flocked substrate of claim 1, wherein said conductive filler comprises a metal, nanoparticles, and/or silver nanoparticles.

9. (canceled)

10. (canceled)

11: The flocked substrate of claim 1, wherein said conductive filler comprises an ionizable salt.

12: The flocked substrate of claim 1, wherein said conductive filler is deposited on the surface of the microfiber or wherein said conductive filler is contained within the microfiber.

13. (canceled)

14: The flocked substrate of claim 4, wherein said adhesive comprises gelatin, chitosan, and/or tannic acid.

15: The flocked substrate of claim 1, wherein said flocked substrate is crosslinked, wherein said flocked substrate is mineralized, wherein said flocked substrate is coated with an absorptive material, and/or wherein said flocked substrate comprises cells and/or a bioactive agent.

16-18. (canceled)

19: The flocked substrate of claim 1, wherein said microfibers are substantially perpendicular to the substrate;
wherein the density of microfibers on the surface of the substrate is greater than about 50 fibers/mm²;
wherein said substrate is coated with an adhesive;
wherein said microfibers have a length of about 200 μm to about 5 mm;
wherein said polymer is an insulative polymer; and
wherein said conductive filler comprises metal nanoparticles and/or ionizable salts.

20: A method of synthesizing a flocked substrate said method comprising flocking microfibers onto a substrate, wherein said microfibers comprise a polymer and a conductive filler, optionally wherein a surface of said substrate is coated with an adhesive.

21: The method of claim 20, wherein said flocking comprises electrostatically flocking.

22: The method of claim 20, further comprising synthesizing said microfibers, further comprising synthesizing the substrate prior to flocking, and/or further comprising applying said adhesive to a surface of the substrate.

23: The method of claim 22, wherein said microfibers are synthesized by wet spinning a microfiber and cutting the wet spun microfiber to the desired length before flocking.

24. (canceled)

25. (canceled)

26: The method of claim 20, wherein said polymer is an insulative polymer.

27: The method of claim 20, wherein said conductive filler comprises a metal, nanoparticles, and/or silver nanoparticles.

28. (canceled)

29. (canceled)

30: The method of claim 20, wherein said conductive filler comprises an ionizable salt.

31: The method of claim 20, further comprising drying and/or thermally curing the flocked substrate, further comprising crosslinking the flocked substrate, further comprising washing and/or sterilizing the flocked substrate, further comprising coating the flocked substrate with an absorptive material, and/or further comprising adding cells and/or a bioactive agent to the flocked substrate.

32-35. (canceled)

36: A method for treating and/or preventing a disease or disorder in a subject in need thereof, said method comprising administering to said subject the flocked substrate of claim 1.

37: The method of claim 36, wherein said disease or disorder is bleeding and said flocked substrates comprise a blood clotting factor.

38: The method of claim 36, wherein said disease or disorder is bone loss and said flocked substrate is mineralized and/or comprises a bone morphogenic protein or fragment of analog thereof.
